(19) United States
(12) Patent Application Publication
(10) Pub. No.: US 2026/0364030 A1
(43) Pub. Date: Nov. 19, 2026

(54) NOVEL SELECTIVE INHIBITORS OF LYSYL OXIDASES

(71) Applicant: Syntara Limited, Frenchs Forest (AU)

(72) Inventors: Wolfgang Jarolimek, Frenchs Forest (AU); Dieter Wolfgang Hamprecht, Frenchs Forest (AU); Alison Dorothy Findlay, Frenchs Forest (AU); Mandar Deodhar, Ashfield (AU)

(73) Assignee: Syntara Limited, Frenchs Forest (AU)

(21) Appl. No.: 19/107,799

(22) PCT Filed: Aug. 29, 2023
(86) PCT No.: PCT/AU2023/050833

§ 371 Date: Feb. 28, 2025

(30) Foreign Application Priority Data
Aug. 29, 2022 (AU) 2022902483

(51) Int. Cl. A61K 31/145; A61K 9/00; A61K 31/277; A61K 31/357; A61K 31/4155; A61K 31/4192; A61K 31/426; A61K 31/55; A61K 47/02; A61K 47/06; A61K 47/10; A61P 17/02; C07B 59/00; C07C 317/28; C07C 317/44; C07D 223/10; C07D 231/12; C07D 249/06; C07D 277/26; C07D 319/18

(52) U.S. Cl. CPC A61K 31/145; A61K 9/0014; A61K 31/277; A61K 31/357; A61K 31/4155; A61K 31/4192; A61K 31/426; A61K 31/55; A61K 47/02; A61K 47/06; A61K 47/10; A61P 17/02; C07B 59/002; C07C 317/28; C07C 317/44; C07D 223/10; C07D 231/12; C07D 249/06; C07D 277/26; C07D 319/18

(57) ABSTRACT

The present invention relates to novel fluoroallylamine sulfone derivatives that are capable of inhibiting certain amine oxidase enzymes. These compounds are useful for the treatment of a variety of indications, e.g., fibrosis, cancer and/or scarring in human subjects as well as in pets and livestock. In addition, the present invention relates to pharmaceutical compositions containing these compounds, as well as uses thereof.

### TECHNICAL FIELD

The present invention relates to novel fluoroallylamine sulfone derivatives that are capable of inhibiting certain amine oxidase enzymes. These compounds are useful for the treatment of a variety of indications, e.g., fibrosis, cancer and/or scarring in human subjects as well as in pets and livestock. In addition, the present invention relates to pharmaceutical compositions containing these compounds, as well as uses thereof.

### BACKGROUND

A family of five closely related enzymes have been linked to fibrotic disease and to metastatic cancer. The enzymes are lysyl oxidase (LOX), the first family member to be described and LOX-like1 (LOXL1), LOXL2, LOXL3, and LOXL4. Lysyl oxidase isoenzymes are copper-dependent amine oxidases which initiate the covalent cross-linking of collagen and elastin. A major function of lysyl oxidase isoenzymes is to facilitate the cross-linking of collagen and elastin by the oxidative deamination of lysine and hydroxylysine amino acid side chains to aldehydes which spontaneously react with neighbouring residues. The resulting cross-linked strands contribute to extracellular matrix (ECM) stability and render it less susceptible to proteolytic degradation by enzymes such as matrix metalloproteases (MMPs). The activity of lysyl oxidase enzymes is crucial for the maintenance of normal tensile and elastic features of connective tissue of many organ systems of the body.

Lysyl oxidase isoenzymes belong to a larger group of amine oxidases which include flavin-dependent and copper-dependent oxidases which are described by the nature of the catalytic co-factor. Flavin-dependent enzymes including monoamine oxidase-A (MAO-A), monoamine oxidase-B (MAO-B), polyamine oxidase and lysine demethylase (LSD1), and the copper-dependent enzymes include semicarbazide sensitive amine oxidase (vascular adhesion protein-1, SSAO/VAP-1), retinal amine oxidase, diamine oxidase and the lysyl oxidase isoenzymes. The copper-dependent amine oxidases have a second co-factor which varies slightly from enzyme to enzyme. In SSAO/VAP-1 it is an oxidized tyrosine residue (TPQ, oxidized to a quinone), whereas in the lysyl oxidase isoenzymes the TPQ has been further processed by addition of a neighbouring lysine residue (to form LTQ).

Lysyl oxidase isoenzymes exhibit different in vivo expression patterns, which suggests that specific isoenzymes will have specific biological roles. Catalytically active forms of LOX have been identified in the cytosolic and nuclear compartments and research is in progress to define their roles in these compartments. LOX itself, for example, plays a major role in epithelial-to-mesenchymal transition (EMT), cell migration, adhesion, transformation and gene regulation. Different patterns of LOX expression/activity have been associated with distinct pathological processes including fibrotic diseases, Alzheimer's disease and other neurodegenerative processes, as well as tumour progression and metastasis.

Directed replacement of dead or damaged cells with connective tissue after injury represents a survival mechanism that is conserved throughout evolution and appears to be most pronounced in humans, serving a valuable role following traumatic injury, infection or diseases. Progressive scarring can occur following more chronic and/or repeated injuries that causes impaired function to parts or the entire affected organ. A variety of causes, such as chronic infections, chronic exposure to alcohol and other toxins, autoimmune and allergic reactions or surgery, radio- and chemotherapy can all lead to fibrosis. This pathological process, therefore, can occur in almost any organ or tissue of the body and, typically, results from situations persisting for several weeks or months in which inflammation, tissue destruction and repair occur simultaneously. In this setting, fibrosis most frequently affects the lungs, liver, skin, kidneys and cardiovascular system.

Liver fibrosis, for example, can occur as a complication of haemochromatosis, Wilson's disease, alcoholism, schistosomiasis, viral hepatitis, bile duct obstruction, exposure to toxins and metabolic disorders. Liver fibrosis is characterized by the accumulation of extracellular matrix that can be distinguished qualitatively from that in normal liver. This fibrosis can progress to cirrhosis, liver failure, cancer and eventually death.

Fibrotic tissues can accumulate in the heart and blood vessels as a result of hypertension, hypertensive heart disease, atherosclerosis and myocardial infarction, where the accumulation of extracellular matrix or fibrotic deposition results in stiffening of the vasculature and stiffening of the cardiac tissue itself.

Pulmonary arterial hypertension (PAH) is a rare and rapidly lethal condition characterised by elevated pulmonary arterial pressure and caused by increased pulmonary vascular resistance. Although a heterogeneous condition with a wide range of causes, there is increasing recognition that PAH is associated with other diseases such as connective-tissue disease and scleroderma. Pathological hallmarks of PAH include vascular wall remodelling with excessive extracellular matrix (ECM) deposition and cross-linking. Lysyl oxidases are dysregulated in pulmonary vasculature of patients with idiopathic pulmonary arterial hypertension (IPAH) and contribute to the persistence of ECM components and improper collagen and elastin remodelling through cross-linking. Prognosis for patients with PAH is poor. Targeting the lysyl oxidases pharmacologically could provide therapeutic intervention where few or none currently exist.

A strong association between fibrosis and increased lysyl oxidase activity has been demonstrated. For example, in experimental hepatic fibrosis in rat, in models of lung fibrosis, in arterial fibrosis, in dermal fibrosis and in adriamycin-induced kidney fibrosis in rat. Of these experimental models of human disease, the most striking increases in enzyme activity were seen in the rat model of CCl₄-induced liver fibrosis. In these studies, the low level of enzyme activity in the healthy liver increased 15- to 30-fold in fibrotic livers.

In humans, there is also a significant association between lysyl oxidase activity measured in the plasma and liver fibrosis progression. Lysyl oxidase activity level is normally low in the serum of healthy subjects, but significantly increased in chronic active hepatitis and even more in cirrhosis. Therefore, lysyl oxidase might serve as a marker of internal fibrosis.

Lysyl oxidase isoenzymes are highly regulated by Hypoxia-Inducible Factor 1α (HIF-1α) and TGF-B, the two most prominent growth factors that cause fibrosis. Collagen cross-linking occurs in every type of fibrosis, hence a lysyl oxidase isoenzyme inhibitor could be used in idiopathic pulmonary fibrosis, scleroderma, kidney or liver fibrosis.

In normal wound healing, granulation tissue formation is a short-lived process, providing a scaffold for re-epithelialisation and repair. Subsequently, the tissue is remodelled and a normotrophic scar is formed. However, after an injury, humans cannot regenerate normal skin. Instead, the repair (or healing) process leads to scar formation (cicatrisation). Scars are both aesthetically and functionally inferior to skin. Scars are a chronic problem and excessive or hypertrophic scarring and its accompanying aesthetic, functional and psychological sequelae remain key challenges for the treatment of deep skin injury and burns. A key factor in the poor appearance and pliability of scars, in particular hypertrophic scars, are the changes to collagen in the dermal layer. In scar tissue the collagen (predominantly Collagen I) is more densely packed and closely aligned in parallel bundles. In normal skin, collagen is not densely packed and is more of a ‘basket-weave’ structure. These alterations, both in structure and quantity of collagen, largely underlie the poor appearance of scar and lead to loss of pliability, discomfort and functional problems.

Dermal fibrosis, or excessive scarring of the skin, is a consequence of exaggerated healing response and is characterized by disproportionate fibroblast proliferation and extracellular matrix (ECM) production in the dermis. Clinically, dermal fibrosis manifests as thickened, tightened and hardened areas of the skin. The spectrum of fibrotic skin disorders is wide, including, but not limited to: hypertrophic scarring, keloids, scleroderma (diffuse and limited subtypes), scleredema (Buschke disease), systemic amyloidosis, lipodermatosclerosis, progeroid disorders, stiff skin syndrome, Dupuytren's contracture, nephrogenic fibrosing dermopathy (NFD), mixed connective tissue disease, scleromyxedema, graft-versus-host disease (GVHD) and eosinophilic fasciitis. Although each of these disorders has its own etiology and clinical characteristics, all involve excessive collagen production, and altered collagen remodelling. One possible mechanism for altered ECM remodelling is through covalent cross-linking. This directly implicates the LOX family of enzymes in the pathogenesis of cutaneous fibrosis. LOX and LOXL1-4 expression is elevated in scar fibroblasts compared to normal skin fibroblasts, with LOX and LOXL1 being the dominant isoforms found in skin tissue.

Keloid, or keloidal scar, is the formation of a type of scar that results from abnormal deposition of granulation tissue (collagen type 3) at the site of a healed skin injury which is then slowly replaced by collagen type 1. This abnormal deposition of collagen, in turn, results from an imbalance in net collagen synthesis and deposition and collagenolysis. Keloids are firm, rubbery lesions or shiny, fibrous nodules, and can vary from pink to the color of the person's skin or red to dark brown in color.

Histologically, keloids are fibrotic tumours characterized by a collection of atypical fibroblasts with excessive deposition of extracellular matrix components, especially collagen, fibronectin, elastin, and proteoglycans. In most cases, they contain relatively acellular centers with thick, abundant collagen bundles that form nodules in the deep dermal region of the lesion. Keloids present a therapeutic challenge, as these lesions can cause significant pain, pruritus, and physical disfigurement. Furthermore, they may not improve in appearance over time and can limit mobility if located over a joint.

Physiologic manipulation of collagen deposition/crosslinking and collagenolysis (through collagenase activity) is, at least theoretically, an opportunity to reduce keloid severity and induce scarring with improved physical properties.

A study on patients with massive, pedunculated keloids has shown that, following excision of the keloid and grafting the defect, treatment by administration of beta aminopropionitrile (BAPN) or penicillamine (non-selective, pan LOX inhibitors) and colchicine (a stimulator of collagenase activity) exerted a measurable beneficial effect on surface scarring.

Studies involving two complimentary, in-vitro skin-like models—human skin equivalent (hSEs), and self-assembled stromal tissues identified LOXL4 as the key isoform mediating TGF-β induced fibrotic phenotypes.

Scarring processes are a considerable problem and challenge in the eye and surrounding structures. Ocular scarring plays a major role in either primary disease (e.g. corneal and conjunctival scarring) or treatment failure (e.g. postoperative trabeculectomy).

Glaucoma is a disease in which the optic nerve is being damaged, leading to progressive and irreversible loss of vision. Elevated intraocular pressure (IOP) is one of the major risk factors for the development and progression of glaucoma. Most treatments for glaucoma are targeted at lowering the intraocular pressure, either by decreasing the formation of aqueous fluid in the eye, or, as in the case of glaucoma filtration surgery, by increasing the outflow of fluid from the eye. Trabeculectomy—the current gold standard for the management of IOP—is a filtering surgery where an ostium is created into the anterior chamber from underneath a partial thickness scleral flap to allow for aqueous flow out of the eye. Post-operative scarring is the main cause of treatment failure. The antimetabolites mitomycin-C(MMC) and 5-fluorouracil (5-FU) are used in current clinical practice to help limit post-operative ocular scar tissue formation. While these agents have been shown to improve the IOP outcome of filtration surgery, they do so in a non-selective manner and are associated with significant side effects. Safer, more targeted, anti-fibrotic agents are needed.

Gingival fibromatosis is a rare and heterogeneous group of disorders that develop as slow progressive, local or diffuse, fibrous enlargements of keratinized gingiva (gingival overgrowth or enlargement). In severe cases, the excess tissue may cover the crowns of the teeth, thus causing masticatory, aesthetic, phonetic, functional and periodontal problems. Gingival overgrowth may be inherited, of idiopathic origin, associated with inflammatory diseases of the oral cavity, or associated with other systemic diseases. However, the majority of cases are due to side-effects of systemic medications such as the anti-seizure drug phenytoin, the immunosuppressant cyclosporin A, and certain anti-hypertensive dihydropyridine anti-calcium-channel-blockers, in particular nifedipine. The pathological manifestation of gingival overgrowth comprises excessive accumulation of extracellular matrix proteins, of which Collagen I is the most predominant. One recognized concept of mechanism for drug induced gingival overgrowth is EMT, a process in which interaction of gingival cells and the extracellular matrix are weakened as epithelial cells transdifferentiate into fibrogenic fibroblast-like cells. The damaged epithelium, basement membrane and underlying stroma result in TGF-β stimulation of lysyl oxidase enzyme activity and contribute to connective tissue fibrosis.

The rationale for the consistent and strong inhibition of fibrosis by lysyl oxidase isoenzyme blockers is that the lack of cross-linking activity renders the collagen susceptible to degradation by proteolytic enzymes such as MMPs. Hence, any type of fibrosis should be reversed by treatment with lysyl oxidase isoenzyme inhibitors. Given the varied involvement of all lysyl oxidase isoenzymes in fibrosis, an inhibitor that demonstrates sustained, strong inhibition of all lysyl oxidase isoenzymes, i.e. a pan LOX inhibitor, should be most efficacious.

Rheumatoid Arthritis (RA) is a systemic autoimmune disorder characterized by chronic, painful inflammation of the lining of the joints. In some people, however, the condition can progress to involve painful swelling and inflammation of the surrounding tissue, and other body systems, including the skin, eyes, lungs, heart and blood vessels. Rheumatoid arthritis is thus a painful and debilitating disease that can result in substantial loss of function and mobility in the hands, wrists and feet. Active rheumatoid arthritis emanates from a few joints, but can subsequently progress to affect multiple joints. Synovial hyperplasia, involving infiltrated immune cells and resident synovial fibroblasts (SFs), is a typical feature of RA. Rheumatoid arthritis synovial fibroblasts (RASFs) are the most common cell type at sites of invasion and are the main culprit in joint destruction. Activated RASFs are able to transmigrate and, as such, have been implicated in the spread of arthritis between joints. Cytokines from the infiltrated immune cells induce activation and proliferation of synovial fibroblasts. These activated SFs in turn generate the pathogenic stroma to perpetuate chronic inflammation, ultimately leading to cartilage and bone destruction. By implanting RASFs together with human cartilage into severe combined immunodeficient mice, it has been demonstrated that activated RASFs migrate in vivo, spreading the disease to the sites of implanted human cartilage. Furthermore, whilst RASFs actively degrade cartilage, controls implanted with synovial fibroblasts from osteoarthritis (OA) patients and cutaneous fibroblasts from healthy donors did not. RASFs differ from unactivated, healthy fibroblasts by their morphology and gene expression. RASFs are characterised by the expression of antiapoptotic, proto-oncogenes and lack of expression of tumour suppressor genes. The production of pro-inflammatory cytokines and chemokines by RASFs further enable attraction of immune cells to the synovium. Furthermore, the production of matrix metalloprotease (MMP) enzymes promotes invasion into and destruction of cartilage.

The type II collagen-induced arthritis (CIA) model is a commonly used animal model for RA as it recapitulates well the signature immunological, pathological and arthritic presentations observed in RA in humans. In CIA rats, high expressions levels of LOX in the synovial membranes, synovial fluid and serum have been demonstrated. Inhibition of LOX with β-aminopropionitrile (BAPN; a pan LOX inhibitor) was found to attenuate inflammation, synovial hyperplasia, angiogenesis and expression of MMP-2 and MMP-9, indicating that LOX promotes synovial hyperplasia and angiogenesis in CIA rats. Furthermore, knockdown of LOXL2 and antibodies against LOXL2 attenuated collagen deposition, proliferation and invasion of RASF.

Whilst there is no cure for RA, there are a number of treatments available that alleviate symptoms and modify disease progression. However, such treatments come with significant side effects associated, in part, with the suppression of the immune system. Selective drugs that target RASF would represent more useful therapy for RA.

Osteoarthritis (OA) is a disease characterised by degeneration of joint cartilage and underlying bone. Predominantly resulting from “wear and tear’, OA causes pain and stiffening of the joint. The most commonly affected joints are those of the fingers, knees, back and hips. Unlike other forms of arthritis (such as RA), osteoarthritis only affects the joints. Often, joints on one side of the body are affected more than those on the other. OA is a progressive and debilitating disease that can have a significant impact on work and normal daily activities.

Synovial fibrosis is a key contributor to OA, and is a manifestation of fibroblast proliferation and an imbalance in collagen synthesis and collagen degradation. This imbalance leads to excessive deposition of collagen into the extracellular matrix (ECM) and results in thickening and stiffening of the synovial membrane.

Genes encoding a number of the lysyl oxidase family of enzymes including LOX, LOXL2, LOXL3 and LOXL4 have been shown to be highly expressed in mice with experimental OA, and humans with end-stage OA.

Given the varied contribution of many of the members of the lysyl oxidase family of enzymes to the development of both rheumatoid arthritis and osteoarthritis, a pan LOX inhibitor may provide for a potentially more efficacious therapy.

BAPN is a widely used, nonselective mechanism-based, irreversible lysyl oxidase inhibitor. Since the 1960s BAPN has been used in animal studies (mainly rat, mouse and hamster) and has been efficacious in reducing collagen content in various models (e.g. CCl₄, bleomycin, quartz, cancer) and tissues (e.g. liver, lung and dermis). However, studies in human patients with scleroderma, found BAPN to be poorly tolerated and highlights the need for safer alternatives.

Lysyl oxidase catalysed collagen cross-linking can proceed via two pathways: the allysine and hydroxyallysine pathways. In the hydroxyallysine pathway, immature divalent crosslinks are formed first, including dehydro-dihydroxylysinonorleucine (deH-DHLNL) and dehydro-hydroxylysinonorleucine (deH-HLNL), and then further progress (via lysyl oxidase independent reactions) to mature trivalent crosslinks, between three collagen molecules to form deoxypyridinoline (DPD) and pyridinoline (PYD). These mature and immature crosslinks can be measured by LC-MS/MS.

Lysyl oxidase isoenzymes are not only involved in the cross-linking of elastin and collagen during wound healing and fibrosis, but also regulate cell movement and signal transduction. Its intracellular and intranuclear function is associated with gene regulation and can lead to tumourigenesis and tumour progression. Both down and upregulation of lysyl oxidase isoenzymes in tumour tissues and cancer cell lines have been described, suggesting a dual role for lysyl oxidase isoenzymes and LOX pro-peptide as a metastasis promoter gene as well as a tumour suppressor gene.

In addition to its role in tissue remodelling, the LOX isoenzymes also play a critical role in primary cancer and metastasis. Tumour growth is associated with a reactive stroma, which is predominantly composed of fibroblasts; termed cancer associated fibroblasts (CAFs). Mice subcutaneously inoculated with an equal mixture of tumour and CAFs cells are known to have a faster growth rate and higher incidence of metastases. CAF knockout models have shown to be pro-tumourigenic, however this is quite an abstract scenario when comparing to a patient's tumour microenvironment. CAFs have been shown to have an increased expression of LOXs compared to normal fibroblasts. Utilising a LOX inhibitor in a cancer setting potentially will affect both the tumour and stromal compartment to assist in decreasing tumour growth and metastasis.

Emerging evidence suggests an association between idiopathic pulmonary fibrosis and lung cancer, however, more studies are needed. Chemical or irradiation induced fibrosis in both, lung and liver mouse models causes an increase in alpha smooth muscle actin (a marker of fibroblasts), LOX expression and metastatic tumour growth, which is reversed by a LOX antibody.

To date, an increase in lysyl oxidase isoenzymes mRNA and/or protein has been observed in breast, CNS cancer cell lines, head and neck squamous cell, esophageal, kidney, lung, prostatic, clear cell renal cell and lung carcinomas, ovarian, uterine, melanoma and osteosarcoma patient samples from The Cancer Genome Atlas (TCGA). Shown in Table 1 is the TCGA patient gene expression data for the LOX family. A plus symbol indicates higher than the average gene expression within this dataset.

TABLE 1
TCGA patient gene expression data for the LOX family

| | | | | | | Number |
|---|---|---|---|---|---|---|
| | | | | | | of patient |
| Cancer | LOX | LOXL1 | LOXL2 | LOXL3 | LOXL4 | samples |
| Breast invasive carcinoma | + | + | + | + | | 1212 |
| Esophageal carcinoma | + | + | + | | | 196 |
| Glioblastoma multiforme | | | + | + | | 171 |
| Head & neck squamous cell | + | + | + | | | 566 |
| carcinoma | | | | | | |
| Kidney renal clear carcinoma | + | | + | | + | 606 |
| Kidney renal papillary cell | | + | | | + | 323 |
| carcinoma | | | | | | |
| Lung squamous cell carcinoma | + | + | + | | + | 552 |
| Mesothelioma | + | + | + | + | + | 87 |
| Ovarian serous cystadenocarcinoma | + | + | | + | + | 307 |
| Pancreatic adenocarcinoma | + | + | + | + | + | 183 |
| Pheochromocytoma and | | | + | + | | 187 |
| paraganglioma | | | | | | |
| Sarcoma | + | + | + | + | | 265 |
| Skin cutaneous melanoma | + | | | + | + | 473 |
| Uterine carcinoma | + | + | + | + | + | 57 |
| Uterine corpus endometrial | | + | | | | 201 |
| carcinoma | | | | | | |

Statistically significant clinical correlations between lysyl oxidase isoenzymes expression and tumour progression have been observed in breast, head and neck squamous cell, myelofibrosis, prostatic, pancreatic, ovarian, and clear cell renal cell carcinomas. The role of lysyl oxidase isoenzymes in tumour progression has been most extensively studied in breast cancer using in vitro models of migration/invasion and in in vivo tumourigenesis and metastasis mouse models. Increased lysyl oxidase isoenzymes expression was found in hypoxic patients, and was associated with negative estrogen receptor status (ER-), decreased overall survival in ER-patients and node-negative patients who did not receive adjuvant systemic treatment, as well as shorter bone metastasis-free survival in ER-patients and node negative patients. In vivo models demonstrated that the LOX inhibitors have potential in breast cancer patients with bone metastasis, by modulating bone homeostasis independent of receptor activator of nuclear factor kappa-B ligand (RANKL). Lysyl oxidase isoenzymes mRNA was demonstrated to be up-regulated in invasive and metastatic cell lines (MDA-MB-231 and Hs578T), as well as in more aggressive breast cancer cell lines and distant metastatic tissues compared with primary cancer tissues.

Pathogenic processes in primary myelofibrosis involve a primary megakaryocyte-weighted clonal myeloproliferation and paraneoplastic stromal reaction that includes bone marrow fibrosis, osteosclerosis, angiogenesis, and extramedullary hematopoiesis. The bone marrow reaction includes excess deposition of extracellular matrix proteins such as fibrillary collagen, hypocellularity, activation and recruitment of bone marrow fibroblasts, excessive cytokine and growth factor production, and other changes that result in a reduction in hematopoietic capacity. Secondary myelofibrosis can result from polycythaemia rubra vera or essential thrombocytosis. In myelofibrosis, disease progression correlates with increased numbers of megakaryocytes, which overexpress LOX. In a GATA 1 low mouse model of myelofibrosis, disease progression (including increase in megakaryocytes number, fibrosis and spleen size), were significantly attenuated by a pan LOX inhibitor.

In most tumour types, the first line of treatment is surgical resection. A wound healing response is initiated by surgery and may correlate with an increase in metastatic spread. Breast cancer models have shown that abdominal surgery increases lung metastasis. Furthermore, it was shown to be caused by systemic LOX. Injection of plasma, collected from abdominal surgery mice (which contained LOX), into tumour bearing mice resulted in an increase in lung metastasis. The surgery induced systemic LOX was blocked by BAPN, reducing metastasis and increasing survival.

In colon, breast cancer and melanoma models, tumour associated endothelial cells have been shown to have an increased expression of LOX, which stimulates angiogenesis and tumour growth.

In pancreatic, breast, lung, ovarian and colon cancer patients, high collagen content has been correlated with high LOX gene expression, chemotherapy resistance and significantly decreased survival. LOX inhibitors (both BAPN and a LOX antibody) and standard of care chemotherapies were combined in desmoplastic tumour mouse models to lower the tumour interstitial pressure causing expansion of vessels. The increased vascular flow increases the concentration of the chemotherapeutic agent at the site of the primary tumour, which leads to a lower metastatic load and increased survival.

In head and neck squamous cell carcinomas, increased lysyl oxidase isoenzyme expression was found in association with CA-IX, a marker of hypoxia, and was associated with decreased cancer specific survival, decreased overall survival and lower metastasis-free survival. In oral squamous cell carcinoma, lysyl oxidase isoenzyme mRNA expression was upregulated compared to normal mucosa.

Gene expression profiling of gliomas identified over-expressed lysyl oxidase isoenzyme as part of a molecular signature indicative of invasion, and associated with higher-grade tumours that are strongly correlated with poor patient survival. Lysyl oxidase isoenzyme protein expression was increased in glioblastoma and astrocytoma tissues, and in invasive U343 and U251 cultured astrocytoma cells.

In tissues, lysyl oxidase isoenzyme mRNA was upregulated in prostate cancer compared to benign prostatic hypertrophy, correlated with Gleason score, and associated with both high grade and short time to recurrence.

In renal clear cell carcinoma (RCC), smoking was associated with allelic imbalances at chromosome 5q23.1, where the LOX gene is localized, and may involve duplication of the gene.

SiHa cervical cancer cells demonstrated increased invasion in vitro under hypoxic/anoxic conditions; this was repressed by inhibition of extracellular catalytically active lysyl oxidase activity by treatment with BAPN as well as LOX antisense oligos, LOX antibody, LOX shRNA or an extracellular copper chelator.

In ovarian cancer genetically engineered mouse models (ApoE knockout) a desmoplastic tumour with increased LOX gene expression is formed. Treatment with BAPN significantly increased survival and decreased lung metastasis. Certain tumours from patients with ovarian cancer have a single nucleotide polymorphism of the LOX gene, G473A. Two independent studies have shown that people with the G473A polymorphism expressed have increased chances of developing ovarian cancer.

In primary human oral squamous cell carcinoma (OSCC), levels of lysyl oxidase enzyme (in particular LOX and LOXL2) and lysyl hydroxylase expression are significantly increased, and markedly elevated in late-stage, regional lymph node metastasis (RLNM)-positive tumours. Both reducible, or immature, cross-links (deH-DHLNL and deH-HLNL) and non-reducible, or mature cross-links (DPD and PYD) are significantly elevated in OSCCs compared to normal tissues.

The findings described herein, provide a strong rationale for combination therapies involving LOX isoenzyme inhibitors and anti-tumour therapy in patients.

More recently, CCT365623 a reversible pan LOX inhibitor has been utilised in breast cancer model (MMTV-PyMT) to reduce metastasis and increase survival.

A person skilled in the art will recognize that drug-like properties are essential to enable drug development. Successful drug development requires finding a compound that combines multiple drug-like properties including, but not limited to:
Physicochemical properties including drug solubility, permeability, lipophilicity and stability
Pharmacological properties including absorption, distribution, metabolism and excretion (ADME); transporter activity; potency at the biological target of interest; duration of action; minimal non-specific and off-target pharmacology
Activity at the desired location
Drug safety; minimal toxicity

Given the complicit role of the lysyl oxidase family of enzymes in the aforementioned diseases, a pan LOX inhibitor with suitable drug-like properties could translate to a clear and significant therapeutic benefit.

The scientific and patent literature describes small molecule inhibitors of lysyl oxidase isoenzymes and antibodies of LOX and LOXL2 with therapeutic effects in animal models of fibrosis and cancer metastasis. Some known MAO inhibitors also are reported to inhibit lysyl oxidase isoenzyme (e.g., the MAO-B inhibitor Mofegiline illustrated below). This inhibitor is a member of the haloallylamine family of MAO inhibitors; the halogen in Mofegiline is fluorine. Fluoroallylamine inhibitors are described in U.S. Pat. No. 4,454,158. There are issued patents claiming fluoroallylamines and chloroallylamines, for example MDL72274 (illustrated below) as inhibitors of lysyl oxidase (U.S. Pat. Nos. 4,943,593; 4,965,288; 5,021,456; 5,059,714; 5,182,297; 5,252,608). Many of the compounds claimed in these patents are also reported to be potent MAO-B and SSAO/VAP-1 inhibitors.

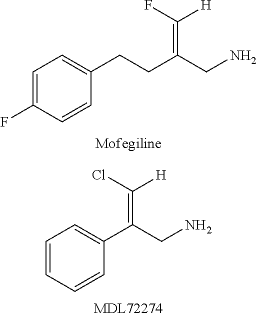 [SMILES: NC/C(=C/F)CCc1ccc(F)cc1 ; NC/C(=C/Cl)c1ccccc1]

Additional fluoroallylamine inhibitors are described U.S. Pat. No. 4,699,928. Other examples structurally related to Mofegiline can be found in WO 2007/120528.

WO 2009/066152 discloses a family of 3-substituted 3-haloallylamines that are inhibitors of SSAO/VAP-1 useful as treatment for a variety of indications, including inflammatory disease. None of these documents specifically disclose the fluoroallylamine compounds according to the present invention.

Antibodies to LOX and LOXL2 have been disclosed in US 2009/0053224 with methods to diagnostic and therapeutic applications. Anti-LOX and anti-LOXL2 antibodies can be used to identify and treat conditions such as a fibrotic condition, angiogenesis, or to prevent a transition from an epithelial cell state to a mesenchymal cell state: US 2011/0044907.

WO 2017/136871 and WO 2017/136870 disclose haloallylamine indole and azaindole derivative inhibitors of lysyl oxidases and uses thereof.

WO 2018/157190 discloses haloallylamine pyrazole derivative inhibitors of lysyl oxidases and uses thereof.

WO 2020/024017 discloses haloallylamine sulfone derivative inhibitors of lysyl oxidases and uses thereof. WO 2021/012014 discloses difluorohaloallylamine sulfone derivative inhibitors of lysyl oxidases.

WO 2017/141049 and WO 2019/073251 disclose families of methylamine and bridged homopiperazine derivatives respectively as lysyl oxidase inhibitors and their use in the treatment of cancer and diseases associated with fibrosis.

WO 2003/097612, WO 2006/053555, and US 2008/0293936 disclose another class of lysyl oxidase inhibitors.

WO 2020/099886, WO 2018/048930, WO 2017/015221, WO 2017/003862, WO 2016/144702 and WO 2016/144703 disclose further LOXL2 inhibitors.

### SUMMARY

The present invention provides novel substituted fluoroallylamine compounds that inhibit lysyl oxidase (LOX), lysyl oxidase-like 2 (LOXL2) and other lysyl oxidase isoenzymes. Surprisingly, these novel 2-sulfonylmethyl-3-fluoroallylamine compounds are potent inhibitors of the human LOX and LOXL isoenzymes. These compounds have been found to have favourable characteristics for topical or oral application. Furthermore, these novel compounds also selectively inhibit certain LOX and LOXL isoenzymes with respect to other enzymes in the amine oxidase family and have a favourable stability and safety profile.

A first aspect of the invention provides for a compound of Formula I:

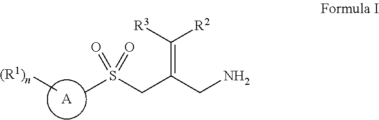

or a pharmaceutically acceptable salt thereof; wherein:
A is aryl;
each R¹ is independently selected from the group consisting of X—R⁴, halogen, deuterium, C₁₋₆alkyl, O—C₁₋₆alkyl, aryl, heteroaryl, cycloalkyl, heterocycloalkyl, —CN, —NO₂, —C(O)OR⁵, —C(O)NR⁶R⁷, —S(O)₂NR⁶R⁷, —S(O)₂R⁸, —NR⁹C(O)R¹⁰, and —NR⁹S(O)₂R¹⁰; wherein each C₁₋₆alkyl, aryl, heteroaryl, cycloalkyl and heterocycloalkyl is optionally substituted by one or more substituents selected from the group consisting of halogen, —OH, and —C₁₋₄alkyl;
R² is H or F:
R³ is H or F; provided that one, and only one, of R² or R³ is F;
X is selected from the group consisting of O, CH₂, and S(O)₂;
R⁴ is selected from the group consisting of aryl and heterocycloalkyl; wherein each aryl is optionally substituted by —S(O)₂R⁸; and wherein each heterocycloalkyl is optionally substituted by ═O;
R⁵ is selected from the group consisting of hydrogen and C₁₋₆alkyl;
R⁶ and R⁷ are independently selected from the group consisting of hydrogen and C₁₋₆alkyl;
R⁸ is C₁₋₆alkyl;
R⁹ is hydrogen or C₁₋₆alkyl;
R¹⁰ is C₁₋₆alkyl; and
n is 0, 1, 2, 3, 4 or 5.

A second aspect of the invention provides for a pharmaceutical composition comprising a compound according to the first aspect of the invention, or a pharmaceutically acceptable salt or solvate thereof, and at least one pharmaceutically acceptable excipient, carrier or diluent.

A third aspect of the invention provides for a method of inhibiting the amine oxidase activity of any one of LOX, LOXL1, LOXL2, LOXL3 and LOXL4 in a subject in need thereof, comprising administering to the subject an effective amount of a compound according to the first aspect of the invention, or a pharmaceutically acceptable salt or solvate thereof, or a pharmaceutical composition according to the second aspect of the invention.

A fourth aspect of the invention provides for a method of treating a condition by inhibiting the activity of any one of the LOX, LOXL1, LOXL2, LOXL3 and LOXL4 proteins, comprising administering to a subject in need thereof a therapeutically effective amount of a compound according to the first aspect of the invention, or a pharmaceutically acceptable salt or solvate thereof, or a pharmaceutical composition according to the second aspect of the invention.

A fifth aspect of the invention provides for use of a compound according to the first aspect of the invention, or a pharmaceutically acceptable salt or solvate thereof, for the manufacture of a medicament for treating a condition by inhibiting the activity of any one of the LOX, LOXL1, LOXL2, LOXL3 and LOXL4 proteins.

A sixth aspect of the invention provides for a compound according to the first aspect of the invention, or a pharmaceutically acceptable salt or, solvate thereof, for use in treating a condition by inhibiting the activity of any one of the LOX, LOXL1, LOXL2, LOXL3 and LOXL4 proteins.

In one embodiment of the methods and uses of the present invention the condition is selected from fibrosis, cancer, and a myeloid malignancy.

In one embodiment of the methods and uses of the present invention the condition is scarring.

A further aspect of the present invention provides for a method of treating or preventing keloid diseases or scars, comprising administering to a subject in need thereof a therapeutically effective amount of the compound or the pharmaceutically acceptable salt thereof according to the present invention.

In one embodiment of the compositions of the present invention, the pharmaceutical composition is a topical composition. In one embodiment of the methods of the present invention the pharmaceutical composition is administered topically.

In one embodiment of the compositions of the present invention, the pharmaceutical composition is an oral composition. In one embodiment of the methods of the present invention the pharmaceutical composition is administered orally.

Contemplated herein is combination therapy in which the methods further comprise co-administering additional therapeutic agents that are used for the treatment of cancer, fibrosis, inflammation, immunosuppression, angiogenesis, fungal infections, bacterial infections, metabolic conditions, pain and pruritis.

### DEFINITIONS

The following are some definitions that may be helpful in understanding the description of the present invention. These are intended as general definitions and should in no way limit the scope of the present invention to those terms alone, but are put forth for a better understanding of the following description.

Unless the context requires otherwise or specifically states to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

As used herein, the term “alkyl” includes within its meaning monovalent (“alkyl”) and divalent (“alkylene”) straight chain or branched chain saturated hydrocarbon radicals having from 1 to 6 carbon atoms, e.g., 1, 2, 3, 4, 5 or 6 carbon atoms. The straight chain or branched alkyl group is attached at any available point to produce a stable compound. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, and the like.

The term “aryl” or variants such as “arylene” as used herein refers to monovalent (“aryl”) and divalent (“arylene”) single, polynuclear, conjugated and fused analogs of aromatic hydrocarbons having from 6 to 10 carbon atoms. A fused analog of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, tetrahydrobenzopyranyl, 1,4-benzodioxanyl, and the like. A “substituted aryl” is an aryl that is independently substituted, with one or more, preferably 1, 2 or 3 substituents, attached at any available atom to produce a stable compound.

The term “heteroaryl” and variants such as “heteroaromatic group” or “heteroarylene” as used herein, includes within its meaning monovalent (“heteroaryl”) and divalent (“heteroarylene”), single, polynuclear, conjugated and fused heteroaromatic radicals having from 5 to 10 atoms, wherein 1 to 4 ring atoms, or 1 to 2 ring atoms are heteroatoms independently selected from O, N, NH and S. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. The heteroaromatic group may be C₁₋₉ heteroaromatic. A fused analog of heteroaryl means a heteroaryl group fused to a monocyclic cycloalkyl or monocyclic heterocyclyl group in which the point of attachment is on the aromatic portion. Examples of heteroaryl groups and fused analogs thereof include pyrazolyl, pyridyl, oxazolyl, oxadiazolyl, thiadiazolyl, tetrazolyl, triazinyl, thienyl, benzoxazolyl, benzothiazolyl, benzimidazolyl, benzofuranyl, benzothiophenyl, furo(2,3-b)pyridyl, indolyl, isoquinolyl, imidazopyridyl, pyrimidinyl, pyridazinyl, pyrazinyl, pyridonyl, phenanthrolinyl, quinolyl, isoquinolinyl, imidazolinyl, thiazolinyl, pyrrolyl, furanyl, thiophenyl, oxazolyl, isoxazolyl, isothiazolyl, triazolyl, and the like. “Nitrogen containing heteroaryl” refers to heteroaryl wherein any heteroatoms are N. A “substituted heteroaryl” is a heteroaryl that is independently substituted, with one or more, preferably 1, 2 or 3 substituents, attached at any available atom to produce a stable compound.

The term “heterocyclyl” and variants such as “heterocycloalkyl” as used herein, includes within its meaning monovalent (“heterocyclyl”) and divalent (“heterocyclylene”), saturated or partially saturated (non-aromatic), monocyclic, bicyclic, polycyclic or fused hydrocarbon radicals having from 3 to 10 ring atoms, wherein from 1 to 4, or from 1 to 2, ring atoms are heteroatoms independently selected from O, N, NH, or S, SO or SO₂, in which the point of attachment may be carbon or nitrogen. A fused analog of heterocyclyl means a monocyclic heterocycle fused to an aryl or heteroaryl group in which the point of attachment is on the non-aromatic portion. The heterocyclyl group may be C₃₋₈ heterocyclyl. The heterocycloalkyl group may be C₃₋₆ heterocyclyl. The heterocyclyl group may be C₃₋₅ heterocyclyl. Examples of heterocyclyl groups and fused analogs thereof include pyrrolidinyl, thiazolidinyl, piperidinyl, piperazinyl, imidazolidinyl, 2,3-dihydrofuro(2,3-b)pyridyl, benzoxazinyl, tetrahydroquinolinyl, tetrahydroisoquinolinyl, dihydroindolyl, quinuclidinyl, azetidinyl, morpholinyl, tetrahydrothiophenyl, tetrahydrofuranyl, tetrahydropyranyl, and the like. The term also includes partially unsaturated monocyclic rings that are not aromatic, such as 2- or 4-pyridones attached through the nitrogen or N-substituted uracils. The heterocycloalkyl may be substituted, for example the heterocycloalkyl may be substituted by ═O.

The term “halogen” or variants such as “halide” or “halo” as used herein refers to fluorine, chlorine, bromine and iodine.

The term “heteroatom” or variants such as “hetero-” or “heterogroup” as used herein refers to O, N, NH and S.

In general, “substituted” refers to an organic group as defined herein (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents.

The term “optionally substituted” as used herein means the group to which this term refers may be unsubstituted, or may be substituted with one or more groups independently selected from alkyl, alkenyl, alkynyl, cycloalkyl, cycloalkenyl, heterocycloalkyl, halo, haloalkyl, hydroxyl, hydroxyalkyl, alkoxy, thioalkoxy, alkenyloxy, haloalkoxy, NO₂, NH(alkyl), N(alkyl)₂, alkylamino, dialkylamino, acyl, alkenoyl, alkynoyl, acylamino, diacylamino, acyloxy, alkylsulfonyl, alkylsulfonyloxy, sulfonamido, heterocycloxy, heterocycloamino, haloheterocycloalkyl, alkylsulfenyl, alkylcarbonyloxy, phosphorus-containing groups such as phosphono and phosphinyl, aryl, heteroaryl, alkylaryl, aralkyl, alkylheteroaryl, cyano, ═O, CO₂H, CO₂alkyl, C(O)NH₂, —C(O)NH(alkyl), and —C(O)N(alkyl)₂. Preferred substituents include halogen, C₁-C₆alkyl, C₁-C₆haloalkyl, C₁-C₆alkoxy, hydroxy(C₁₋₆)alkyl, C₃-C₆cycloalkyl, C(O)OH, NHC(O)C₁-C₄alkyl, C(O)C₁-C₄alkyl, NH₂, NHC₁-C₄alkyl, N(C₁-C₄alkyl)₂, SO₂(C₁-C₄alkyl), OH and CN. Particularly preferred substituents include C₁₋₄alkyl, C₁₋₄alkoxy, SO₂(C₁-C₄alkyl), ═O, halogen, OH, hydroxy(C₁₋₃)alkyl (e.g. C(CH₃)₂OH), and C₁₋₃haloalkyl (e.g. CF₃, CH₂CF₃).

The present invention includes within its scope all stereoisomeric and isomeric forms of the compounds disclosed herein, including all diastereomeric isomers, racemates, enantiomers and mixtures thereof. It is also understood that the compounds described by Formula I may be present as E and Z isomers, also known as cis and trans isomers. Thus, the present disclosure should be understood to include, for example, E, Z, cis, trans, (R), (S), (L), (D), (+), and/or (−) forms of the compounds, as appropriate in each case. Where a structure has no specific stereoisomerism indicated, it should be understood that any and all possible isomers are encompassed. Compounds of the present invention embrace all conformational isomers. Compounds of the present invention may also exist in one or more tautomeric forms, including both single tautomers and mixtures of tautomers. Also included in the scope of the present invention are all polymorphs and crystal forms of the compounds disclosed herein.

The present invention includes within its scope isotopes of different atoms. Any atom not specifically designated as a particular isotope is meant to represent any stable isotope of that atom. Thus, the present disclosure should be understood to include the deuterium isotope of hydrogen.

All references cited in this application are specifically incorporated by cross-reference in their entirety. Reference to any such documents should not be construed as an admission that the document forms part of the common general knowledge or is prior art.

In the context of this specification the term “administering” and variations of that term including “administer” and “administration”, includes contacting, applying, delivering or providing a compound or composition of the invention to an organism, or a surface by any appropriate means.

In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

In the context of this specification the term “topical administration” or variations on that term including “topical application” includes within its meaning applying, contacting, delivering or providing a compound or composition of the invention to the skin, or localized regions of the body.

In the context of this specification the term “local administration” or variations on that term including “local application” includes within its meaning applying, contacting, delivering or providing a compound or composition of the invention to the skin, or localized regions of the body.

In the context of this specification the term “effective amount” includes within its meaning a sufficient but non-toxic amount of a compound or composition of the invention to provide a desired effect. Thus, the term “therapeutically effective amount” includes within its meaning a sufficient but non-toxic amount of a compound or composition of the invention to provide the desired therapeutic effect. The exact amount required will vary from subject to subject depending on factors such as the species being treated, the sex, age and general condition of the subject, the severity of the condition being treated, the particular agent being administered, the mode of administration, and so forth. Thus, it is not possible to specify an exact “effective amount”.

However, for any given case, an appropriate “effective amount” may be determined by one of ordinary skill in the art using only routine experimentation.

### BRIEF DESCRIPTION OF THE FIGURES

FIG. 1: a) depicts the time dependent inhibitory effects of Compound 1 against LOXL1; b) depicts the time dependent inhibitory effects of Compound 1 against LOXL2.

FIG. 2 depicts the diffusion of Compound 1 through human skin.

FIG. 3 depicts efficacy of Compound 1 in a mouse model of sclerosis (bleo=bleomycin; ns=no significance; ****=P≤0.0001; Cmpd 1=Compound 1).

FIG. 4 depicts reduction in collagen cross-links following treatment with Compound 1 in a rodent injury model (*=P≤0.05).

### DETAILED DESCRIPTION

The present invention relates to novel fluoroallylamine sulfone derivatives which may inhibit lysyl oxidase (LOX), lysyl oxidase-like2 (LOXL2) and other lysyl oxidase isoenzymes.

In particular, the present invention relates to compounds of Formula I:

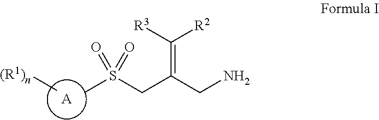

or a pharmaceutically acceptable salt thereof; wherein:
A is aryl;
each R¹ is independently selected from the group consisting of X—R⁴, halogen, deuterium, C₁₋₆alkyl, O—C₁₋₆alkyl, aryl, heteroaryl, cycloalkyl, heterocycloalkyl, —CN, —NO₂, —C(O)OR⁵, —C(O)NR⁶R⁷, —S(O)₂NR⁶R⁷, —S(O)₂R⁸, —NR⁹C(O)R¹⁰, and —NR⁹S(O)₂R¹⁰; wherein each C₁₋₆alkyl, aryl, heteroaryl, cycloalkyl and heterocycloalkyl is optionally substituted by one or more substituents selected from the group consisting of halogen, —OH, and —C₁₋₄alkyl;
R² is H or F;
R³ is H or F; provided that one, and only one, of R² or R³ is F;
X is selected from the group consisting of O, CH₂, and S(O)₂;
R⁴ is selected from the group consisting of aryl and heterocycloalkyl; wherein each aryl is optionally substituted by —S(O)₂R⁸; and wherein each heterocycloalkyl is optionally substituted by ═O;
R⁵ is selected from the group consisting of hydrogen and C₁₋₆alkyl;
R⁶ and R⁷ are independently selected from the group consisting of hydrogen and C₁₋₆alkyl;
R⁸ is C₁₋₆alkyl;
R⁹ is hydrogen or C₁₋₆alkyl;
R¹⁰ is C₁₋₆alkyl; and
n is 0, 1, 2, 3, 4 or 5.

In some embodiments, the present invention includes polymorphic forms, solvates, hydrates or tautomeric forms of the compounds of Formula I. In some embodiments, the present invention includes polymorphic forms of the compounds of Formula I. In other embodiments the present invention includes solvates or hydrates of the compounds of Formula I. In further embodiments the present invention includes tautomeric forms of the compounds of Formula I.

In one embodiment of compounds of the present invention, A is selected from the group consisting of phenyl, naphthyl and 1,4-benzodioxanyl. In another embodiment of compounds of the present invention, A is phenyl.

In one embodiment of compounds of the present invention, R¹ is independently selected from the group consisting of X—R⁴, halogen, deuterium, C₁₋₆alkyl, O—C₁₋₆alkyl, aryl, heteroaryl, cycloalkyl, heterocycloalkyl, —CN, —NO₂, —C(O)OR⁵, —C(O)NR⁶R⁷, —S(O)₂NR⁶R⁷, —S(O)₂R⁸, —NR⁹C(O)R¹⁰, and —NR⁹S(O)₂R¹⁰; wherein each C₁₋₆alkyl, aryl, heteroaryl, cycloalkyl and heterocycloalkyl is optionally substituted by one or more substituents selected from the group consisting of halogen, —OH, and —C₁₋₄alkyl. In another embodiment of compounds of the present invention, each R¹ is independently selected from the group consisting of X—R⁴, halogen, deuterium, C₁₋₆alkyl, O—C₁₋₆alkyl, heteroaryl, —CN, —NO₂, —C(O)OR⁵, —C(O)NR⁶R⁷ and —S(O)₂R⁸; wherein each C₁₋₆alkyl and heteroaryl is optionally substituted by one or more substituents selected from the group consisting of halogen and —C₁₋₄alkyl. In a further embodiment of compounds of the present invention, each R¹ is independently selected from the group consisting of X—R⁴, halogen, deuterium, —C₁₋₄alkyl, —OCH₃, heteroaryl, —CN, —NO₂, —C(O)OH, —C(O)NH₂, —S(O)₂CH₃, wherein each —C₁₋₄alkyl and heteroaryl is optionally substituted by one or more substituents selected from the group consisting of fluorine and —C₁₋₄alkyl. In another embodiment of compounds of the present invention, each R¹ is independently selected from the group consisting of X—R⁴, F, Cl, Br, deuterium, -methyl, —CF₃, isopropyl, —OCH₃, thiazolyl, pyrazolyl, —CN, —NO₂, —C(O)OH, —C(O)NH₂, —S(O)₂CH₃, wherein each thiazolyl or pyrazolyl is optionally substituted by methyl.

In one embodiment of compounds of the present invention, R² is H or F and R³ is H or F, provided that one, and only one of R² or R³ is F. In another embodiment of the compounds of the present invention R² is F and R³ is H. In a further embodiment of the compounds of the present invention R² is H and R³ is F.

In one embodiment of the compounds of the present invention X is selected from the group consisting of O, CH₂, and S(O)₂. In another embodiment of the compounds of the present invention X is selected from the group consisting of O and S(O)₂. In another embodiment of the compounds of the present invention X is O. In a further embodiment of the compounds of the present invention X is CH₂. In another embodiment of the compounds of the present invention X is S(O)₂.

In one embodiment of the compounds of the present invention R⁴ is selected from the group consisting of aryl and heterocycloalkyl; wherein each aryl is optionally substituted by —S(O)₂R⁸, and wherein each heterocycloalkyl is optionally substituted by ═O. In another embodiment of the compounds of the present invention, R⁴ is selected from the group consisting of phenyl and

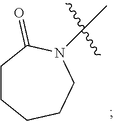 [SMILES: *N1CCCCCC1=O]

wherein each phenyl is optionally substituted by —S(O)₂CH₃. In a further embodiment R⁴ is phenyl. In another embodiment R⁴ is phenyl substituted by —S(O)₂CH₃. In a further embodiment R⁴ is

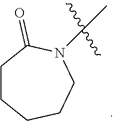 [SMILES: *N1CCCCCC1=O]

In one embodiment of the compounds of the present invention R⁵ is selected from the group consisting of hydrogen and C₁₋₆alkyl. In another embodiment of the compounds of the present invention R⁵ is selected from the group consisting of hydrogen and methyl. In a further embodiment of the compounds of the present invention R⁵ is hydrogen. In another embodiment of the compounds of the present invention R⁵ is methyl.

In one embodiment of the compounds of the present invention R⁶ and R⁷ are independently selected from the group consisting of hydrogen and C₁₋₆alkyl. In another embodiment of the compounds of the present invention R⁶ and R⁷ are both hydrogen. In a further embodiment of the compounds of the present invention R⁶ and R⁷ are both methyl.

In one embodiment of the compounds of the present invention R⁸ is C₁₋₆alkyl. In another embodiment of the compounds of the present invention R⁸ is methyl.

In one embodiment of the compounds of the present invention R⁹ is hydrogen or C₁₋₆alkyl.

In another embodiment of the compounds of the present invention R⁹ is hydrogen. In a further embodiment of the compounds of the present invention R⁹ is C₁₋₆alkyl. In another embodiment of the compounds of the present invention R⁹ is methyl.

In one embodiment of the compounds of the present invention R¹⁰ is C₁₋₆alkyl. In another embodiment of the compounds of the present invention R¹⁰ is methyl.

In one of embodiment of compounds of the present invention, n is 0, 1, 2, 3, 4 or 5. In another embodiment of compounds of the present invention, n is 0. In a further embodiment of compounds of the present invention, n is 0, 1 or 2. In another embodiment of compounds of the present invention, n is 1, 2 or 3. In another embodiment of compounds of the present invention n is 1 or 2. In a further embodiment of compounds of the present invention, n is 1. In another embodiment of compounds of the present invention, n is 2. In a further embodiment of compounds of the present invention, n is 3. In another embodiment of compounds of the present invention, n is 4. In a further embodiment of compounds of the present invention, n is 5.

In one embodiment, the present invention relates to a compound of Formula Ia:

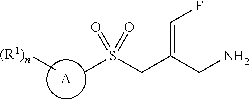

or a pharmaceutically acceptable salt thereof, wherein A, R¹ and n are as defined for compounds of Formula I.

In one embodiment, the present invention relates to a compound of Formula Ib:

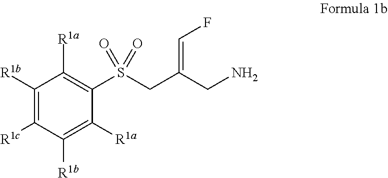

or a pharmaceutically acceptable salt thereof; wherein:
each R^{1a} is independently selected from the group consisting of X—R⁴, halogen, deuterium, C₁₋₄alkyl and —CN;
each Rib is independently selected from the group consisting of X—R⁴, halogen, deuterium, —C₁₋₄alkyl, —O—C₁₋₄alkyl, heteroaryl, —CN, —NO₂, —C(O)OR⁵, —C(O)NR⁶R⁷, —S(O)₂R⁸, wherein each —C₁₋₄alkyl and heteroaryl is optionally substituted by one or more substituents selected from the group consisting of halogen, and —C₁₋₄alkyl;
R^{1c} is selected from the group consisting of deuterium, —C₁₋₄alkyl, —CN, —C(O)OR⁵ and —S(O)₂R⁸;
X is selected from the group consisting of O, CH₂, and S(O)₂;
R⁴ is selected from the group consisting of aryl and heterocycloalkyl; wherein each R⁴ is optionally substituted by —S(O)₂R⁸;
R⁵ is selected from the group consisting of hydrogen and C₁₋₄alkyl;
R⁶ and R⁷ are independently selected from the group consisting of hydrogen and C₁₋₄alkyl; and
R⁸ is C₁₋₄alkyl.

In one embodiment of compounds of Formula Ib, each R^{1a} is independently selected from the group consisting of X—R⁴, chlorine, deuterium, methyl and —CN; each R^{1b} is independently selected from the group consisting of X—R⁴, halogen, deuterium, —C₁₋₄alkyl, —OCH₃, heteroaryl, —CN, —NO₂, —C(O)OH, —C(O)NH₂, —S(O)₂CH₃, wherein each —C₁₋₄alkyl and heteroaryl is optionally substituted by one or more substituents selected from the group consisting of fluorine, and —C₁₋₄alkyl; R^{1c} is selected from the group consisting of deuterium, methyl, —CN, —C(O)OH and —S(O)₂CH₃; X is selected from the group consisting of O, CH₂, and S(O)₂; and R⁴ is selected from the group consisting of phenyl and

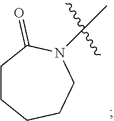 [SMILES: *N1CCCCCC1=O]

wherein each phenyl is optionally substituted by —S(O)₂CH₃.

In a further embodiment of compounds of Formula Ib, each R^{1a} is independently selected from the group consisting of X—R⁴, chlorine, deuterium, methyl and —CN; each R^{1b} is independently selected from the group consisting of (X—R⁴, F, Cl, Br, deuterium, -methyl, —CF₃, isopropyl, —OCH₃, thiazolyl, pyrazolyl, triazolyl —CN, —NO₂, —C(O)OH, —C(O)NH₂, —S(O)₂CH₃, wherein each thiazolyl or pyrazolyl is optionally substituted by methyl; R^{1c} is selected from the group consisting of deuterium, methyl, —CN, —C(O)OH and —S(O)₂CH₃; X is selected from the group consisting of O, CH₂, and S(O)₂; and R⁴ is selected from the group consisting of phenyl and

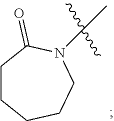 [SMILES: *N1CCCCCC1=O]

wherein each phenyl is optionally substituted by —S(O)₂CH₃.

Exemplary compounds according to the present invention include the compounds set forth in Table 2:

TABLE 2

| | | |
|---|---|---|
| 1 | 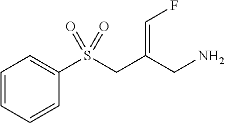 [SMILES: NC/C(=C\F)CS(=O)(=O)c1ccccc1] | (E)-3-fluoro-2-((phenylsulfonyl)methyl)prop- 2-en-1-amine |
| 2 | 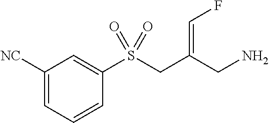 [SMILES: N#Cc1cccc(S(=O)(=O)C/C(=C/F)CN)c1] | (E)-3-((2-(aminomethyl)-3- fluoroallyl)sulfonyl)benzonitrile |
| 3 | 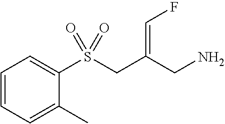 [SMILES: Cc1ccccc1S(=O)(=O)C/C(=C/F)CN] | (E)-3-fluoro-2-((o-tolylsulfonyl)methyl)prop- 2-en-1-amine |
| 4 | 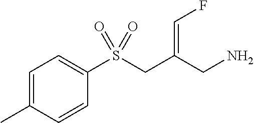 [SMILES: Cc1ccc(S(=O)(=O)C/C(=C/F)CN)cc1] | (E)-3-fluoro-2-(tosylmethyl)prop-2-en-1- amine |
| 5 | 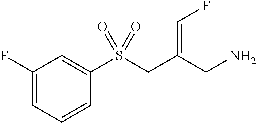 [SMILES: NC/C(=C\F)CS(=O)(=O)c1cccc(F)c1] | (E)-3-fluoro-2-(((3- fluorophenyl)sulfonyl)methyl)prop-2-en-1- amine |
| 6 | 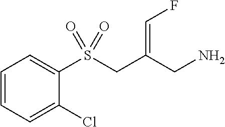 [SMILES: NC/C(=C\F)CS(=O)(=O)c1ccccc1Cl] | (E)-2-(((2-chlorophenyl)sulfonyl)methyl)-3- fluoroprop-2-en-1-amine |
| 7 | 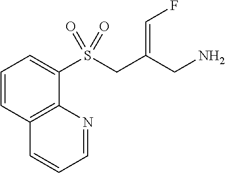 [SMILES: NC/C(=C\F)CS(=O)(=O)c1cccc2cccnc12] | (E)-3-fluoro-2-((quinolin-8- ylsulfonyl)methyl)prop-2-en-1-amine |
| 8 | 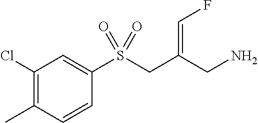 [SMILES: Cc1ccc(S(=O)(=O)C/C(=C/F)CN)cc1Cl] | (E)-2-(((3-chloro-4- methylphenyl)sulfonyl)methyl)-3-fluoroprop- 2-en-1-amine |
| 9 | 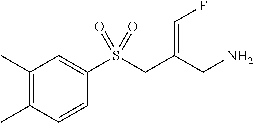 [SMILES: Cc1ccc(S(=O)(=O)C/C(=C/F)CN)cc1C] | (E)-2-(((3,4- dimethylphenyl)sulfonyl)methyl)-3- fluoroprop-2-en-1-amine |
| 10 | 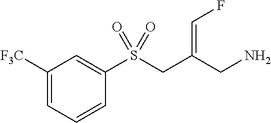 [SMILES: NC/C(=C\F)CS(=O)(=O)c1cccc(C(F)(F)F)c1] | (E)-3-fluoro-2-(((3- (trifluoromethyl)phenyl)sulfonyl)methyl) prop-2-en-1-amine |
| 11 | 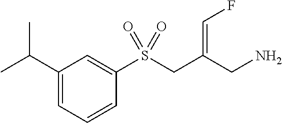 [SMILES: CC(C)c1cccc(S(=O)(=O)C/C(=C/F)CN)c1] | (E)-3-fluoro-2-(((3- isopropylphenyl)sulfonyl)methyl)prop-2-en- 1-amine |
| 12 | 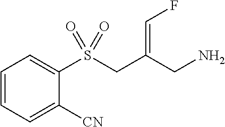 [SMILES: N#Cc1ccccc1S(=O)(=O)C/C(=C/F)CN] | (E)-2-((2-(aminomethyl)-3- fluoroallyl)sulfonyl)benzonitrile |
| 13 | 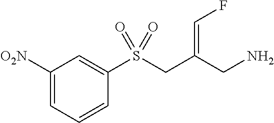 [SMILES: NC/C(=C\F)CS(=O)(=O)c1cccc([N+](=O)[O-])c1] | (E)-3-fluoro-2-(((3- nitrophenyl)sulfonyl)methyl)prop-2-en-1- amine |
| 14 | 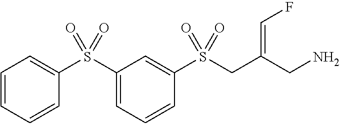 [SMILES: NC/C(=C\F)CS(=O)(=O)c1cccc(S(=O)(=O)c2ccccc2)c1] | (E)-3-fluoro-2-(((3- (phenylsulfonyl)phenyl)sulfonyl)methyl) prop-2-en-1-amine |
| 15 | 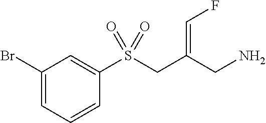 [SMILES: NC/C(=C\F)CS(=O)(=O)c1cccc(Br)c1] | (E)-2-(((3-bromophenyl)sulfonyl)methyl)-3- fluoroprop-2-en-1-amine |
| 16 | 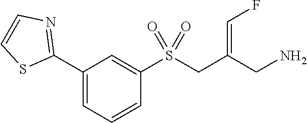 [SMILES: NC/C(=C\F)CS(=O)(=O)c1cccc(-c2nccs2)c1] | (E)-3-fluoro-2-(((3-(thiazol-2- yl)phenyl)sulfonyl)methyl)prop-2-en-1-amine |
| 17 | 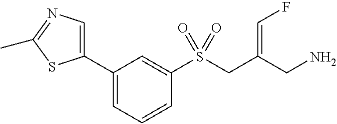 [SMILES: Cc1ncc(-c2cccc(S(=O)(=O)C/C(=C/F)CN)c2)s1] | (E)-3-fluoro-2-(((3-(2-methylthiazol-5- yl)phenyl)sulfonyl)methyl)prop-2-en-1-amine |
| 18 | 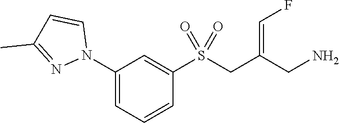 [SMILES: Cc1ccn(-c2cccc(S(=O)(=O)C/C(=C/F)CN)c2)n1] | (E)-3-fluoro-2-(((3-(3-methyl-1H-pyrazol-1- yl)phenyl)sulfonyl)methyl)prop-2-en-1-amine |
| 19 | 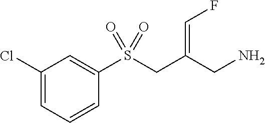 [SMILES: NC/C(=C\F)CS(=O)(=O)c1cccc(Cl)c1] | (E)-2-(((3-chlorophenyl)sulfonyl)methyl)-3- fluoroprop-2-en-1-amine |
| 20 | 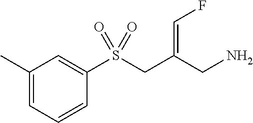 [SMILES: Cc1cccc(S(=O)(=O)C/C(=C/F)CN)c1] | (E)-3-fluoro-2-((m- tolylsulfonyl)methyl)prop-2-en-1-amine |
| 21 | 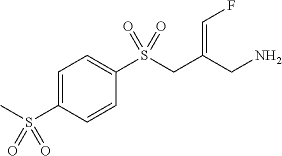 [SMILES: CS(=O)(=O)c1ccc(S(=O)(=O)C/C(=C/F)CN)cc1] | (E)-3-fluoro-2-(((4- (methylsulfonyl)phenyl)sulfonyl)methyl) prop-2-en-1-amine |
| 22 | 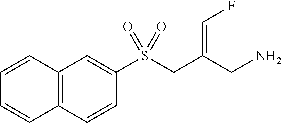 [SMILES: NC/C(=C\F)CS(=O)(=O)c1ccc2ccccc2c1] | (E)-3-fluoro-2-((naphthalen-2- ylsulfonyl)methyl)prop-2-en-1-amine |
| 23 | 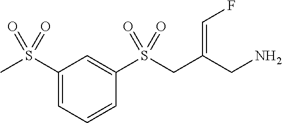 [SMILES: CS(=O)(=O)c1cccc(S(=O)(=O)C/C(=C/F)CN)c1] | (E)-3-fluoro-2-(((3- (methylsulfonyl)phenyl)sulfonyl)methyl) prop-2-en-1-amine |
| 24 | 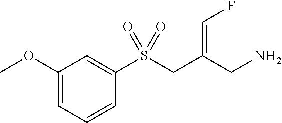 [SMILES: COc1cccc(S(=O)(=O)C/C(=C/F)CN)c1] | (E)-3-fluoro-2-(((3- methoxyphenyl)sulfonyl)methyl)prop-2-en-1- amine |
| 25 | 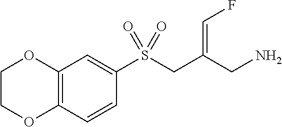 [SMILES: NC/C(=C\F)CS(=O)(=O)c1ccc2c(c1)OCCO2] | (E)-2-(((2,3-dihydrobenzo[b][1,4]dioxin-6- yl)sulfonyl)methyl)-3-fluoroprop-2-en-1- amine |
| 26 | 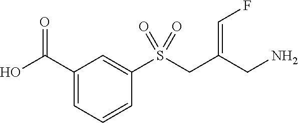 [SMILES: NC/C(=C\F)CS(=O)(=O)c1cccc(C(=O)O)c1] | (E)-3-((2-(aminomethyl)-3- fluoroallyl)sulfonyl)benzoic acid |
| 27 | 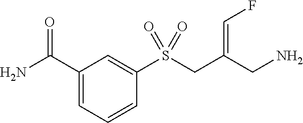 [SMILES: NC/C(=C\F)CS(=O)(=O)c1cccc(C(N)=O)c1] | (E)-3-((2-(aminomethyl)-3- fluoroallyl)sulfonyl)benzamide |
| 28 | 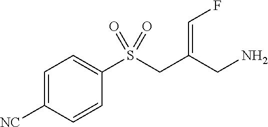 [SMILES: N#Cc1ccc(S(=O)(=O)C/C(=C/F)CN)cc1] | (E)-4-((2-(aminomethyl)-3- fluoroallyl)sulfonyl)benzonitrile |
| 29 | 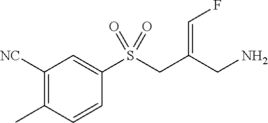 [SMILES: Cc1ccc(S(=O)(=O)C/C(=C/F)CN)cc1C#N] | (E)-5-((2-(aminomethyl)-3- fluoroallyl)sulfonyl)-2-methylbenzonitrile |
| 30 | 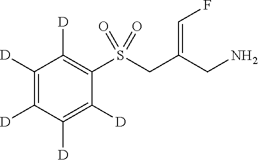 [SMILES: [2H]c1c([2H])c([2H])c(S(=O)(=O)C/C(=C/F)CN)c([2H])c1[2H]] | (E)-3-fluoro-2-(((phenyl- d₅)sulfonyl)methyl)prop-2-en-1-amine |
| 31 | 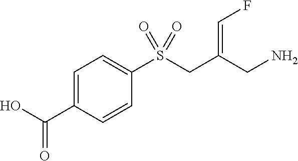 [SMILES: NC/C(=C\F)CS(=O)(=O)c1ccc(C(=O)O)cc1] | (E)-4-((2-(aminomethyl)-3- fluoroallyl)sulfonyl)benzoic acid |
| 32 | 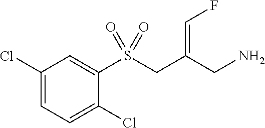 [SMILES: NC/C(=C\F)CS(=O)(=O)c1cc(Cl)ccc1Cl] | (E)-2-(((2,5-dichlorophenyl)sulfonyl)methyl)- 3-fluoroprop-2-en-1-amine |
| 33 | 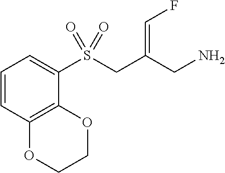 [SMILES: NC/C(=C\F)CS(=O)(=O)c1cccc2c1OCCO2] | (E)-2-(((2,3-dihydrobenzo[b][1,4]dioxin-5- yl)sulfonyl)methyl)-3-fluoroprop-2-en-1- amine |
| 34 | 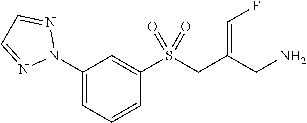 [SMILES: NC/C(=C\F)CS(=O)(=O)c1cccc(-n2nccn2)c1] | (E)-2-(((3-(2H-1,2,3-triazol-2- yl)phenyl)sulfonyl)methyl)-3-fluoroprop-2- en-1-amine |
| 35 | 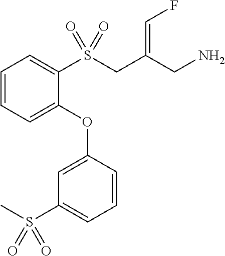 [SMILES: CS(=O)(=O)c1cccc(Oc2ccccc2S(=O)(=O)C/C(=C/F)CN)c1] | (E)-3-fluoro-2-(((2-(3- (methylsulfonyl)phenoxy)phenyl)sulfonyl) methyl)prop-2-en-1-amine |
| 36 | 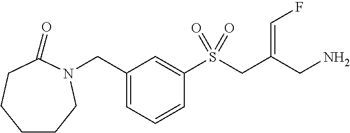 [SMILES: NC/C(=C\F)CS(=O)(=O)c1cccc(CN2CCCCCC2=O)c1] | (E)-1-(3-((2-(aminomethyl)-3- fluoroallyl)sulfonyl)benzyl)azepan-2-one |
| 37 | 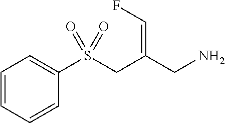 [SMILES: NC/C(=C/F)CS(=O)(=O)c1ccccc1] | (Z)-3-fluoro-2-((phenylsulfonyl)methyl)prop- 2-en-1-amine |

or a pharmaceutically acceptable salt thereof.

In particular, the present invention relates to the compound:

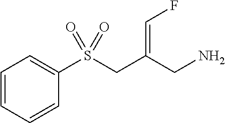 [SMILES: NC/C(=C\F)CS(=O)(=O)c1ccccc1]

or a pharmaceutically acceptable salt, polymorphic form, solvate, hydrate or tautomeric form thereof.

In one embodiment, the compound is a pharmaceutically acceptable salt, polymorphic form, solvate, hydrate or tautomeric form of the compound.

In one embodiment, the compound is a pharmaceutically acceptable salt of the compound.

In another embodiment, the compound is the hydrochloride salt:

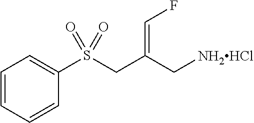

The one embodiment, the compound of the present invention has the chemical name (E)-3-fluoro-2-((phenylsulfonyl)methyl)prop-2-en-1-amine.

### Preparation of Compounds of Formula I

Compounds described herein are synthesized using standard synthetic techniques using methods known in the art.

Compounds are prepared using standard organic techniques described, but not limited to, Advanced Organic Chemistry, 6th Edition by March, John Wiley and Sons Inc. Standard procedures for the use of protecting groups for the temporary protection of functional groups such as alcohols, amines and carboxylic acids are described in for example Protecting Groups in Organic Synthesis, 4th Edition, John Wiley and Sons Inc.

Alternative reaction conditions for the chemical transformations described herein may be employed such as variation in solvent, reaction temperature, reaction time as well as different chemical reagents.

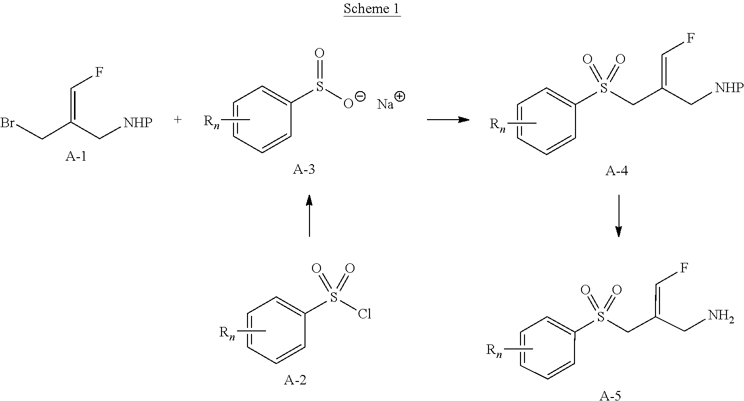

P is a functional group used to protect a nitrogen functionality. Examples of P are carbonates such as the tert-butyloxycarbonyl (BOC), the 9-fluorenylmethyloxycarbonyl (FMOC), and the benzyloxycarbonyl (CBZ) groups. In some embodiments, allylbromide A-1 (the synthesis of A-1 is described in patents WO 2021/258159 and WO 2018/158140) is treated with arylsulfinic acid, sodium salt A-3 in a suitable solvent such as DMF to give allylsulfone A-4. In some embodiments, the solvent is acetone or acetonitrile. In some embodiments, A-3 can be prepared from the corresponding arylsulfonyl chloride A-2 by treatment with sodium sulphite and sodium carbonate in a suitable solvent such as water. In other embodiments, the reaction is conducted over a period of 1-5 h. In some embodiments, standard amine deprotection conditions afford A-5. In some embodiments, the deprotection conditions involve treatment of A-4 with HCl solution. In other embodiments, HCl in 1,4-dioxane is used. In some embodiments, HCl in diethyl ether is used. In some embodiments, HCl in ethyl acetate is used. In other embodiments, the deprotection conditions involve treatment of A-4 with TFA.

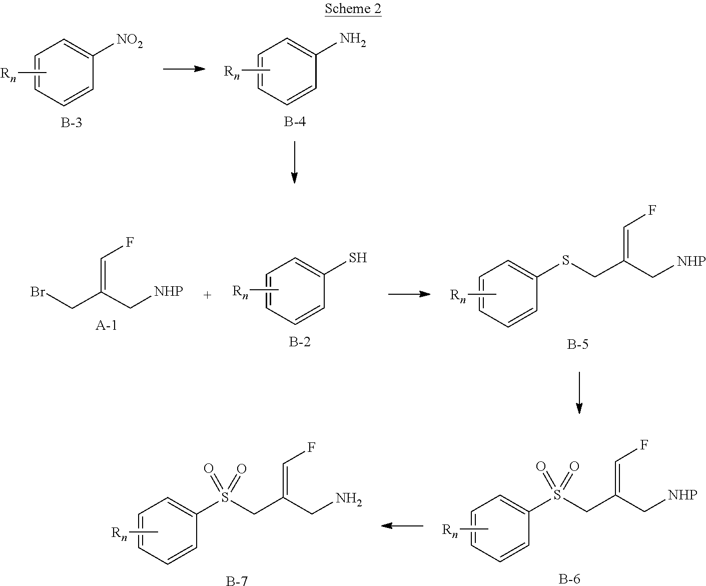

In some embodiments, thiol derivative B-2 is treated with allyl bromide A-1 in the presence of a suitable base using a suitable polar solvent to afford thioether derivative B-5. In some embodiments, the base is Cs₂CO₃. In other embodiments, the base is K₂CO₃. In some embodiments, the solvent is DMF. In other embodiments, the solvent is acetone or alternatively, acetonitrile. In some embodiments, sulfone derivative B-6 is obtained by treatment of thioether B-5 with a suitable oxidising agent in a suitable solvent. In some embodiments, the oxidising agent is hydrogen peroxide. In other embodiments, sodium tungstate is added. In yet other embodiments, the oxidising agent is Oxone® (potassium peroxymonosulfate). In some embodiments, the solvent is methanol. In some embodiments, standard amine deprotection conditions afford B-7. In some embodiments, the deprotection conditions involve treatment of B-6 with HCl solution. In other embodiments HCl in 1,4-dioxane is used. In other embodiments, the deprotection conditions involve treatment of B-6 with TFA.

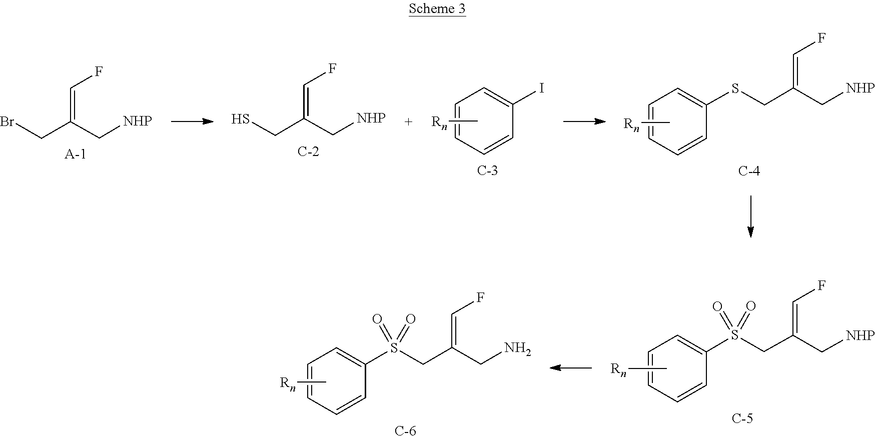

In some embodiments, allylsulfide derivative C-2 can be obtained from A-1 by treatment with thiourea followed by base promoted hydrolysis in a suitable solvent at a suitable temperature.

In some embodiments, the solvent is methanol. In some embodiments, the temperature is reflux. In other embodiments the temperature is rt. In some embodiments, allylsulfide C-2 can be coupled with aryliodide C-3 in the presence of a suitable catalyst and base to afford allylsulfide C-4. In some embodiments, the catalyst is CuO. In other embodiments, the catalyst is CuI or alternatively a palladium containing catalyst. In some embodiments, the base is potassium hydroxide. In other embodiments, the base is Cs₂CO₃. In some embodiments, the solvent used for the coupling reaction is DMSO. In other embodiments, the solvent is water or alternatively, DMF. In some embodiments, the temperature at which the coupling reaction is conducted is 120 °C. In some embodiments, sulfone derivative C-5 is obtained by treatment of thioether C-4 with a suitable oxidising agent in a suitable solvent. In some embodiments, the oxidising agent is hydrogen peroxide. In other embodiments, the sodium tungstate is added. In yet other embodiments, the oxidising agent is Oxone®. In some embodiments, the solvent is methanol. In some embodiments, standard amine deprotection conditions afford C-6. In some embodiments, the deprotection conditions involve treatment of C-5 with HCl solution. In other embodiments, HCl in 1,4-dioxane is used. In other embodiments, the deprotection conditions involve treatment of C-5 with TFA.

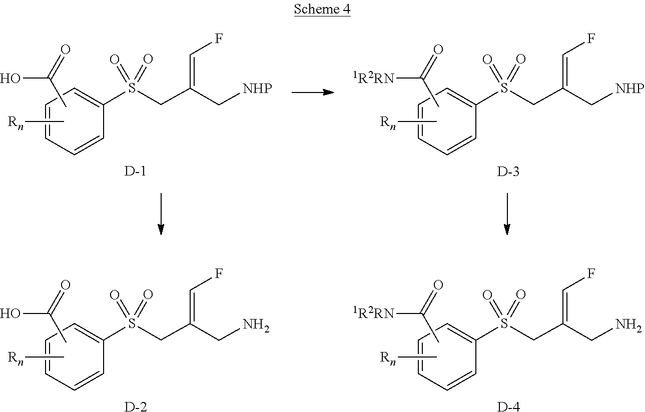

In some embodiments, acid derivative D-1 is converted to the corresponding amide D-3 using standard peptide coupling techniques known to a person in the art. In some embodiments, standard peptide coupling conditions between carboxylic acid derivatives D-1 and an appropriately substituted amine HNR¹R² affords amides D-3. In some embodiments, standard amine deprotection conditions afford D-4 from D-3, and D-2 from D-1. In some embodiments, the deprotection conditions involve treatment of D-1 or D-3 with HCl solution. In other embodiments, HCl in 1,4-dioxane is used. In other embodiments, the deprotection conditions involve treatment of D-1 or D-3 with TFA.

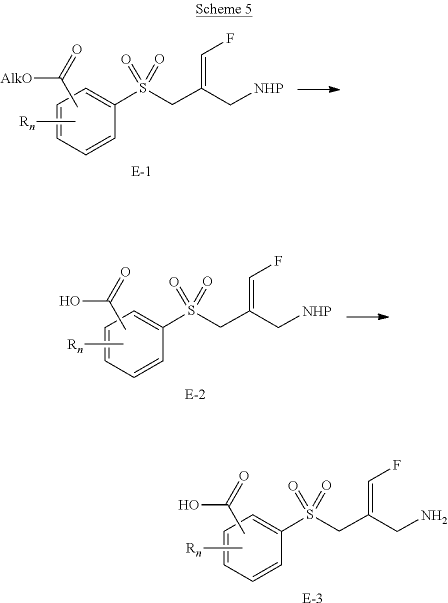

In some embodiments, ester E-1 is converted to corresponding acid derivatives E-2 by treatment with a suitable base in water and a suitable co-solvent or co-solvents. In some embodiments, the base is sodium hydroxide. In other embodiments, the base is potassium hydroxide. In some embodiments, the co-solvent used is methanol. In other embodiments, the co-solvents are methanol and THF. In some embodiments, standard amine deprotection conditions afford E-3. In some embodiments, the deprotection conditions involve treatment of E-2 with HCl solution. In other embodiments HCl in 1,4-dioxane is used. In other embodiments, the deprotection conditions involve treatment of E-2 with TFA.

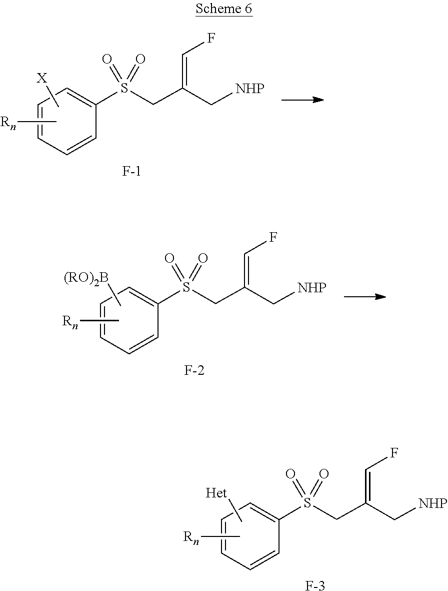

In some embodiments, boronic ester F-2 is obtained from aryl halide by treatment with a suitable diboron reagent, palladium based catalyst and base in a suitable solvent. In some embodiments, the diboron reagent is bis(pinacolato)diboron. In some embodiments, the palladium catalyst is bis(diphenylphosphino)ferrocene-palladium(II)dichloride dichloromethane complex. In some embodiments, the base is potassium acetate. In some embodiments, the solvent is 1,4-dioxane. In some embodiments, biaryl derivative F-3 is obtained by application of standard Suzuki-type coupling conditions between boronic ester F-2 and a suitably substituted aryl or heteroaryl. In some embodiments, the palladium catalyst employed for the coupling reaction is tetrakis(triphenylphosphine)palladium(0). In some embodiments, the base used is potassium phosphate tribasic. In other embodiments, the solvent used is 1,4-dioxane. In yet other embodiments, the temperature at which the coupling reaction is conducted is 90 °C. In some embodiments, biaryl derivative F-3 is obtained from boronic ester F-2 and a suitably substituted aryl or heteroaryl by treatment with copper acetate and a suitable base. In some embodiments, the base is pyridine. In other embodiments, the reaction is conducted in a solvent such as acetonitrile.

Cis/trans (E/Z) isomers may be separated by conventional techniques well known to those skilled in the art, for example, chromatography and fractional crystallisation.

### Therapeutic Uses and Formulations

Another aspect of the present invention relates to a pharmaceutical composition comprising a compound of the invention, or a pharmaceutically acceptable salt thereof, together with a pharmaceutically acceptable diluent, excipient or adjuvant.

The present invention also relates to use of the compounds of the invention in therapy, in particular to inhibit members of the lysyl oxidase family members, LOX, LOXL1, LOXL2, LOXL3 and LOXL4. In one embodiment, the invention provides for the selective inhibition of specific lysyl oxidase isoenzymes. In another embodiment, the invention provides for the simultaneous inhibition of 2, 3, 4 or 5 lysyl oxidase isoenzymes. The relative inhibitory potencies of the compound can be determined by the amount needed to inhibit the amine oxidase activity of LOX, LOXL1, LOXL2, LOXL3 and LOXL4 in a variety of ways, e.g., in an in vitro assay with recombinant or purified human protein or with recombinant or purified non-human enzyme, in cellular assays expressing normal rodent enzyme, in cellular assays which have been transfected with human protein, in in vivo tests in rodent and other mammalian species, and the like.

In one embodiment, the compounds of the present invention are long lasting inhibitors of the lysyl oxidase family members LOX, LOXL1, LOXL2, LOXL3 and LOXL4. In one embodiment, the compound of the present invention are long lasting inhibitors of the LOX or LOXL1-4 enzymes if the inhibition continues to be greater than 50% of the LOX or LOXL1-4 enzymes' activity after the compounds concentration has been reduced below the IC₅₀. In one embodiment, the compounds of the present invention exhibit sustained inhibition of the LOX or LOXL1-4 enzymes over a period of 30 minutes. In one embodiment, the compounds of the present invention exhibit sustained inhibition of the LOX or LOXL1-4 enzymes over a period of 24 hours. In one embodiment, the compounds of the present invention are irreversible inhibitors of the lysyl oxidase family members LOX, LOXL1, LOXL2, LOXL3 and LOXL4.

Accordingly, a further aspect of the invention is directed to a method of inhibiting the amine oxidase activity of any one of LOX, LOXL1, LOXL2, LOXL3 or LOXL4 in a subject in need thereof, comprising administering to the subject an effective amount of a compound of the invention, or a pharmaceutically acceptable salt or solvate thereof, or a pharmaceutical composition thereof.

In one embodiment, the present invention is directed to a method of inhibiting the amine oxidase activity of LOXL2. In another embodiment, the present invention is directed towards inhibiting the amine oxidase activity of LOX and LOXL2. In a further embodiment, the present invention is directed to a method of inhibiting the amine oxidase activity of LOX.

As discussed previously, LOX and LOXL1-4 enzymes are members of a large family of flavin-dependent and copper-dependent amine oxidases, which includes SSAO/VAP-1, monoamine oxidase-B (MAO-B) and diamine oxidase (DAO). In one embodiment, the compounds of the present invention selectively inhibits members of the lysyl oxidase isoenzyme family with respect to SSAO/VAP-1, MAO-B or DAO. In one embodiment, the compounds of the present invention selectively inhibit members of the lysyl oxidase isoenzyme family with respect to other members of the amine oxidase family.

Compounds containing an amine moiety may be metabolised as a substrate by amine oxidases. This may reduce the therapeutically effective compound concentration and generate noxious products. The compounds of the present invention are stable in the presence of SSAO/VAP-1, MAO-A, MAO-B, DAO and dog serum.

The present invention also discloses methods to use the compounds of the invention to inhibit one or more lysyl oxidase isoenzymes (LOX, LOXL1, LOXL2, LOXL3 and LOXL4) in patients suffering from a fibrotic disease, and methods to treat fibrotic diseases. Furthermore, the present invention discloses methods to use the compounds of the invention to inhibit one or more lysyl oxidase isoenzymes (LOX, LOXL1, LOXL2, LOXL3 and LOXL4) in patients suffering from cancer, including metastatic cancer, and methods to treat cancer and metastatic cancer.

In a further aspect of the invention, there is provided a method of treating a condition by inhibiting the activity of any one of the LOX, LOXL1, LOXL2, LOXL3 and LOXL4 proteins, comprising administering to a subject in need thereof a therapeutically effective amount of the compounds of the invention, or a pharmaceutically acceptable salt or solvate thereof, or a pharmaceutical composition thereof.

In another aspect, there is a provided a method of treating a condition modulated by any one of LOX, LOXL1, LOXL2, LOXL3 and LOXL4, comprising administering to a subject in need thereof a therapeutically effective amount of the compounds of the invention, or a pharmaceutically acceptable salt or solvate thereof, or a pharmaceutical composition thereof.

In one embodiment of the methods of the present invention, the condition is selected from the group consisting of fibrosis, cancer and arthritis.

In another aspect, the present invention provides a method for decreasing extracellular matrix formation by treating human subjects, pets and livestock with the fluoroallylamine inhibitor of the lysyl oxidase isoenzyme family of the invention as described herein.

The above-described methods are applicable wherein the condition is fibrosis. As employed here “fibrosis” includes such diseases as cystic fibrosis, pulmonary fibrosis, liver fibrosis, kidney fibrosis, scleroderma, radiation-induced fibrosis, Dupuytren's contracture, Peyronie's disease, scarring and other diseases where excessive fibrosis contributes to disease pathology.

In one embodiment, the fibrosis is selected from the group consisting of mediastinal fibrosis, myelofibrosis, retroperitoneal fibrosis, progressive massive fibrosis, nephrogenic systemic fibrosis, Crohn's Disease, keloid, scleroderma/systemic sclerosis, arthrofibrosis, Dupuytren's contracture, adhesive capsulitis, fibrosis of the pancreases, fibrosis of the intestine, liver fibrosis, lung fibrosis, kidney fibrosis, cardiac fibrosis, fibrostenosis, cystic fibrosis, idiopathic pulmonary fibrosis, radiation-induced fibrosis, Peyronie's disease and scleroderma or is associated with respiratory disease, abnormal wound healing and repair, scarring, hypertrophic scarring/keloids, scarring post-surgery, cardiac arrest and all conditions where excess or aberrant deposition of fibrous material is associated with disease, injury, implants or surgery. In another embodiment, the fibrosis is selected from the group consisting of liver fibrosis, lung fibrosis, kidney fibrosis, cardiac fibrosis, scarring and scleroderma. In a further embodiment the fibrosis is selected from the group consisting of keloid, scarring, hypertrophic scarring, scleroderma and Dupuytren's contracture.

In one embodiment, kidney fibrosis includes, but is not limited to, diabetic nephropathy, vesicoureteral reflux, tubulointerstitial renal fibrosis; glomerulonephritis or glomerular nephritis, including focal segmental glomerulosclerosis and membranous glomerulonephritis, IgA nephropathy and mesangiocapillary glomerular nephritis. In one embodiment, liver fibrosis results in cirrhosis, and includes associated conditions such as chronic viral hepatitis, non-alcoholic fatty liver disease (NAFLD), alcoholic steatohepatitis (ASH), non-alcoholic steatohepatitis (NASH), primary biliary cirrhosis (PBC), biliary cirrhosis, and autoimmune hepatitis.

In one embodiment, the fibrosis is selected from keloid, scarring, ocular scarring, hypertrophic scarring, scleroderma, Dupuytren's contracture and Peyronie's disease. In one embodiment, the hypertrophic scarring results from a burn. In one embodiment, the hypertrophic scarring is caused by external injuries. In another embodiment, the hypertrophic scarring is caused by surgical procedures. In one embodiment, the keloid is caused by external injuries. In another embodiment, the keloid is caused by surgical procedures. In a further embodiment, the keloid is a result of a skin injury caused by acne, burns, chicken pox, piercing, scratches, surgical cuts or vaccination sites.

A further aspect of the present invention provides for a method of treating or preventing keloid diseases or scars, comprising administering to a subject in need thereof a therapeutically effective amount of the compound or the pharmaceutically acceptable salt thereof according to the present invention. In one embodiment the compound is administered topically. In another embodiment, the compound is administered orally.

In one embodiment, the keloid diseases or scars are keloids, hypertrophic scars, postoperative scars, burn scars, posttraumatic scars, Dupuytren's contracture or are caused by external injuries or surgical procedures or are a result of a skin injury caused by acne, burns, chicken pox, infection, piercing, scratches, surgical cuts or vaccination sites. In one embodiment, the treatment assists in wound healing and improves improves the skin compliance or skin appearance of the subject.

The above-described methods are also applicable wherein the condition is a proliferative disease for example cancer. In one embodiment, the cancer is selected from the group consisting of lung cancer; breast cancer; colorectal cancer; anal cancer; pancreatic cancer; prostate cancer; ovarian carcinoma; liver and bile duct carcinoma; esophageal carcinoma; non-Hodgkin's lymphoma; bladder carcinoma; carcinoma of the uterus; glioma, glioblastoma, medullablastoma, and other tumours of the brain; myelofibrosis, kidney cancer; cancer of the head and neck; cancer of the stomach; multiple myeloma; testicular cancer; germ cell tumour; neuroendocrine tumour; cervical cancer; oral cancer, carcinoids of the gastrointestinal tract, breast, and other organs; signet ring cell carcinoma; mesenchymal tumours including sarcomas, fibrosarcomas, haemangioma, angiomatosis, haemangiopericytoma, pseudoangiomatous stromal hyperplasia, myofibroblastoma, fibromatosis, inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma or a leiomyosarcoma.

In one embodiment, the proliferative disease is a myeloid malignancy selected from the group consisting of myelodysplastic syndromes (MDS), myelodysplastic neoplasma (MPN), MDS/MPS overlap syndromes and acute myeloid leukemia (AML), including acute promyelocytic leukemia (APL).

In one embodiment, the cancer is selected from the group consisting of breast cancer, head and neck squamous cell carcinoma, brain cancer, prostate cancer, renal cell carcinoma, liver cancer, lung cancer, oral cancer, cervical cancer and tumour metastasis.

In one embodiment, lung cancer includes lung adenocarcinoma, squamous cell carcinoma, large cell carcinoma, bronchoalveolar carcinoma, non-small-cell carcinoma, small cell carcinoma and mesothelioma. In one embodiment breast cancer includes ductal carcinoma, lobular carcinoma, inflammatory breast cancer, clear cell carcinoma, and mucinous carcinoma. In one embodiment, colorectal cancer includes colon cancer and rectal cancer. In one embodiment, pancreatic cancer includes pancreatic adenocarcinoma, islet cell carcinoma and neuroendocrine tumours.

In one embodiment, ovarian carcinoma includes ovarian epithelial carcinoma or surface epithelial-stromal tumour including serous tumour, endometrioid tumour and mucinous cystadenocarcinoma, and sex-cord-stromal tumour. In one embodiment liver and bile duct carcinoma includes hepatocelluar carcinoma, cholangiocarcinoma and hemangioma. In one embodiment, esophageal carcinoma includes esophageal adenocarcinoma and squamous cell carcinoma. In one embodiment, carcinoma of the uterus includes endometrial adenocarcinoma, uterine papillary serous carcinoma, uterine clear-cell carcinoma, uterine sarcomas and leiomyosarcomas and mixed mullerian tumours. In one embodiment, kidney cancer includes renal cell carcinoma, clear cell carcinoma and Wilm's tumour. In one embodiment, cancer of the head and neck includes squamous cell carcinomas. In one embodiment, cancer of the stomach includes stomach adenocarcinoma and gastrointestinal stromal tumour.

In one embodiment, the cancer is selected from the group consisting of pancreatic cancer, liver cancer, breast cancer, myelofibrosis and mesothelioma.

In one embodiment, the compounds of the invention may be for use in the treatment of a non-metastatic cancer. In another embodiment, the compounds of the invention may be for use in the treatment of metastatic cancer. In a further embodiment, the compound of the present invention may be for use in the prevention or treatment of tumour metastasis.

The above-described methods are applicable wherein the condition is arthritis. As used herein arthritis includes rheumatoid arthritis and osteoarthritis.

In one embodiment of the methods of the present invention, the subject is selected from the group consisting of humans, pets and livestock. In another embodiment of the methods of the present invention, the subject is a human.

A further aspect of the invention provides for use of a compound of the invention, or a pharmaceutically acceptable salt or solvate thereof, for the manufacture of a medicament for treating a condition by inhibiting the activity of any one of the LOX, LOXL1, LOXL2, LOXL3 and LOXL4 proteins.

Another aspect of the invention provides for use of a compound of the invention, or a pharmaceutically acceptable salt or solvate thereof, for the manufacture of a medicament for treating a condition modulated by any one of LOX, LOXL1, LOXL2, LOXL3 and LOXL4.

Pharmaceutical and/or Therapeutic Formulations

In another embodiment of the present invention, there are provided compositions comprising a compound of the invention and at least one pharmaceutically acceptable excipient, carrier or diluent thereof. The compounds of the invention may also be present as suitable salts, including pharmaceutically acceptable salts.

The phrase “pharmaceutically acceptable carrier” refers to any carrier known to those skilled in the art to be suitable for the particular mode of administration. In addition, the compounds may be formulated as the sole pharmaceutically active ingredient in the composition or may be combined with other active ingredients.

The phrase “pharmaceutically acceptable salt” refers to any salt preparation that is appropriate for use in a pharmaceutical application. By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art and include acid addition and base salts. Hemisalts of acids and bases may also be formed. Pharmaceutically acceptable salts include amine salts of mineral acids (e.g., hydrochlorides, hydrobromides, sulfates, and the like); and amine salts of organic acids (e.g., formates, acetates, lactates, malates, tartrates, citrates, ascorbates, succinates, maleates, butyrates, valerates, fumarates, sulfonates and the like).

For compounds having a basic site, suitable pharmaceutically acceptable salts may be acid addition salts. For example, suitable pharmaceutically acceptable salts of such compounds may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the invention.

S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Representative acid addition salts include acetate, adipate, alginate, ascorbate, asparate, benzenesulfonate, benzoate, bisulfate, borate, butyrate, camphorate, camphorsulfonate, citrate, digluconate, cyclopentanepropionate, dodecylsulfate, ethanesulfonate, fumarate, glucoheptonate, glycerophosphate, hemisulfate, heptonate, hexanoate, hydrobromide, hydrochloride, 2-hydroxy-ethanesulfonate, lactobionate, lactate, laurate, lauryl sulfate, malate, maleate, malonate, methanesulfonate, 2-naphthalenesulfonate, nicotinate, oleate, palmitate, pamoate, pectinate, 3-phenylpropionate, phosphate, pivalate, propionate, stearate, succinate, sulfate, tartrate, toluenesulfonate, undecanoate, valerate salts, and the like. Suitable base salts are formed from bases that form non-toxic salts. Examples include the arginine, benzathine, calcium, choline, diethylamine, diolamine, glycine, lysine, magnesium, meglumine, olamine, potassium, sodium, tromethamine and zinc salts. Representative alkali or alkaline earth metal salts include sodium, lithium, potassium, calcium, magnesium, and the like, as well as non-toxic ammonium, quaternary ammonium, and amine cations, including, but not limited to ammonium, tetramethylammonium, tetraethylammonium, dimethylamine, trimethylamine, triethylamine, triethanolamine and the like.

Pharmaceutically acceptable salts of the compound may be prepared by methods known to those skilled in the art, including for example:
(i) by reacting the compound with the desired acid or base;
(ii) by removing an acid- or base-labile protecting group from a suitable precursor of the compound or by ring-opening a suitable cyclic precursor, for example, a lactone or lactam, using the desired acid or base; or
(iii) by converting one salt of the compound to another by reaction with an appropriate acid or base or by means of a suitable ion exchange column.

The above reactions (i)-(iii) are typically carried out in solution. The resulting salt may precipitate out and be collected by filtration or may be recovered by evaporation of the solvent. The degree of ionisation in the resulting salt may vary from completely ionised to almost non-ionised.

Thus, for instance, suitable pharmaceutically acceptable salts of compounds according to the present invention may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, carbonic acid, tartaric acid, or citric acid with the compounds of the invention. Suitable pharmaceutically acceptable salts of the compounds of the present invention therefore include acid addition salts.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Compositions herein comprise the compounds provided herein. In one embodiment, a compound of the invention is formulated into suitable pharmaceutical preparations such as solutions, suspensions, tablets, creams, gels, dispersible tablets, pills, capsules, powders, sustained release formulations or elixirs, for oral administration or in sterile solutions or suspensions for parenteral administration, as well as transdermal patch preparation and dry powder inhalers. In one embodiment, the compound is formulated into pharmaceutical compositions using techniques and procedures well known in the art.

In the compositions, effective concentrations the compound or pharmaceutically acceptable derivatives thereof is (are) mixed with a suitable pharmaceutical carrier. The compound may be derivatized as the corresponding salts, base, solvates, hydrates prior to formulation, as described above. The concentrations of the compound in the compositions is effective for delivery of an amount, upon administration, that treats, prevents, or ameliorates one or more of the symptoms of diseases or disorders to be treated.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein, and then extrapolated from there for dosages for humans.

The concentration of active compound in the pharmaceutical composition will depend on absorption, distribution, inactivation and elimination rates of the active compound, the physicochemical characteristics of the compound, the dosage schedule, and amount administered as well as other factors known to those of skill in the art.

Dosing may occur at intervals of minutes, hours, days, weeks, months or years or continuously over any one of these periods. Suitable dosages lie within the range of about 0.1 ng per kg of body weight to 0.1 g per kg of body weight per dosage. The dosage is preferably in the range of 1 μg to 0.1 g per kg of body weight per dosage, such as is in the range of 1 mg to 0.1 g per kg of body weight per dosage. Suitably, the dosage is in the range of 1 μg to 50 mg per kg of body weight per dosage, such as 1 μg to 20 mg per kg of body weight per dosage, or 1 μg to 10 mg per kg of body weight per dosage. Other suitable dosages may be in the range of 1 mg to 25 mg per kg of body weight, including 1 mg to 10, 20, 50 or 100 mg per kg of body weight per dosage or 10 μg to 100 mg per kg of body weight per dosage. In one embodiment, the dosage is in the range of 1 mg to 10 mg per kg of body weight per dosage.

Alternatively, an effective dosage may be up to about 10 mg/cm², or it may be up to about 1 mg/cm², about 0.5 mg/cm², about 0.2 mg/cm², about 0.1 mg/cm², about 0.05 mg/cm², about 0.02 mg/cm², or about 0.01 mg/cm². It may be, for example, in the range from about 0.1 pg/cm² to about 1 mg/cm², or from about 1 pg/cm² to about 1 mg/cm², about 10 pg/cm² to about 1 mg/cm², about 10 μg/cm² to about 0.1 mg/cm², about 10 μg/cm² to about 0.01 mg/cm², about 10 μg/cm² to about 500 μg/cm², about 10 μg/cm² to about 200 μg/cm², about 10 μg/cm² to about 100 μg/cm², about 10 μg/cm² to about 50 μg/cm², about 20 μg/cm² to about 1 mg/cm², about 50 μg/cm² to about 1 mg/cm², about 100 μg/cm² to about 1 mg/cm², about 200 μg/cm² to about 1 mg/cm², about 500 μg/cm² to about 1 mg/cm², about 50 μg/cm² to about 500 μg/cm², about 50 μg/cm² to about 200 μg/cm², about 100 μg/cm² to about 500 μg/cm², or about 200 μg/cm² to about 500 μg/cm².

Suitable dosage amounts and dosing regimens can be determined by the attending physician and may depend on the particular condition being treated, the severity of the condition, as well as the general health, age and weight of the subject.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, dissolution in aqueous sodium bicarbonate, formulating the compounds of interest as nanoparticles, and the like. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof.

The pharmaceutically therapeutically active compounds and derivatives thereof are, in one embodiment, formulated and administered in unit-dosage forms or multiple-dosage forms. The active ingredient may be administered at once, or may be divided into a number of smaller doses to be administered at intervals of time. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules. Hence, multiple dose form is a multiple of unit-doses which are not segregated in packaging.

Actual methods of preparing such dosage forms are known, or will be apparent, to those skilled in this art.

Dosage forms or compositions containing active ingredient in the range of 0.005% to 100% (wt %) with the balance made up from non-toxic carrier may be prepared. Methods for preparation of these compositions are known to those skilled in the art. The contemplated compositions may contain 0.001%-100% (wt %) active ingredient, in one embodiment 0.1-95% (wt %), in another embodiment 75-85% (wt %) and in another embodiment 0.1-25%. (wt %) active ingredient. The amount of active in such therapeutically useful compositions is such that an effective dosage level can be attained.

### Modes of Administration

Convenient modes of administration include injection (subcutaneous, intravenous, etc.), oral administration, inhalation, transdermal application, topical to skin, eyes, ears, oral surfaces, vaginal or rectal administration. Depending on the route of administration, the formulation and/or compound may be coated with a material to protect the compound from the action of enzymes, acids and other natural conditions which may inactivate the therapeutic activity of the compound. The compound may also be administered parenterally or intraperitoneally.

### Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

### Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and ethanol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

### Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, olive oil, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4 °C to rt.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

### Topical Administration

A topical composition typically includes an active, a pharmaceutically acceptable carrier and optionally one or more additional ingredients that, for example, aid in the formation of the desired delivery vehicle of the active. Topical mixtures are prepared as described for the local and systemic administration. The resulting mixture may be a solution, suspension, emulsion or the like and are formulated as a cream, gel, jelly, wax, ointment, emulsion, solution, elixir, lotion, suspension, tincture, paste, foam, aerosol, irrigation, spray, suppository, bandage, dermal patch, and/or a combination thereof or any other formulations suitable for topical administration.

### Carriers

Active agents can be combined with a pharmaceutically acceptable carrier or diluent to produce a pharmaceutical composition or formulation. In such pharmaceutical formulations, the active agents or therapeutic composition can be combined with a “carrier” that is physiologically compatible with the skin or mucosal tissue of a human or animal to which it is topically administered. Typically, the carrier is substantially inactive, with the exception of its intrinsic sur-factant properties which may aid in the production of a solution or suspension of the active ingredients. The compositions may include other physiologically active constituents that do not interfere with the efficacy of the active agents in the composition. In some embodiments, the carriers can be liquid or gel-based materials for use in liquid or gel formulations. The specific formulations depend, in part, upon the desired routes or modes of administration.

Suitable carrier materials include any carrier or vehicle commonly used as a base for solutions, dispersions, emulsions, gels, creams, ointment, lotions, pastes, or foams, for topical administration. Examples include emulsifying agents, inert carriers including hydrocarbon bases, emulsifying bases, non-toxic solvents or water-soluble bases.

Many suitable liquid or gel-based carriers are well known in the art. The carrier should be able to dissolve or disperse an active at an effective level, optionally with the aid of non-toxic surfactants. Examples include water, physiological salt solutions, alcohols (e.g., methanol, ethanol, propanol, or butanol), glycerol, glycols (e.g., ethylene glycol, propylene glycol, or ethoxy diglycol), polyethylene glycol (e.g., MW 400 to 20,000), water-alcohol/glycol blends, and the like. Suitable carriers and diluents for certain embodiments include, for example, water, saline, isotonic saline solutions, for example, phosphate-buffered saline, aqueous dextrose, glycerol, ethoxy diglycol, dimethyl sulfoxide (DMSO), and the like, or combinations thereof.

Suitable carriers further include aqueous and oleaginous carriers such as, for example, white petrolatum, isopropyl myristate, lanolin or lanolin alcohols, mineral oil, fragrant or essential oil, nasturtium extract oil, sorbitan mono-oleate, cetostearyl alcohol (togetheror in various combinations), and detergents (e.g., polysorbates (Tweens) such as polysorbate 20, 40, 60, or 80; polyoxyl stearate; or sodium lauryl sulfate). One or more carrier materials can be mixed with water to form a lotion, gel, cream, semi-solid composition, or the like. Other suitable carriers include water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether, water, or combinations thereof. For example, emulsions containing water, glycerol stearate, glycerin, mineral oil, synthetic spermaceti, cetyl alcohol, or combinations thereof, may be used. Preservatives may also be included in the carrier, such as one or more of butylparaben, methylparaben, propylparaben, benzyl alcohol, and ethylene diamine tetraacetate salts. The composition of the carrier can be varied so long as it does not interfere significantly with the pharmacological activity of the active ingredients of the therapeutic composition.

Suitable pharmaceutically acceptable carriers include, but are not limited to, creams such as Cetaphil Moisturising Cream (Galderma Laboratories, L.P.), QV Cream (Lision Hong), Sorbolene, or the like. In some embodiments, the pharmaceutically acceptable carrier includes a lotion, such as Alpha Keri Moisturizing Lotion (Mentholatum), DermaVeen Moisturizing Lotion (DermaTech Laboratories), QV Skin Lotion (Lision Hong), Cetaphil Moisturizing Lotion (Galderma Laboratories, L.P.), or the like.

### Gelling Agents and Thickening Agents

The compositions described herein can include one or more gelling agents to increase the viscosity of the composition. Examples of gelling agents and thickening agents, include, but are not limited to, fatty acids, fatty acid salts and esters, fatty alcohols, synthetic polymers, modified celluloses, xanthangum, or combinations thereof. Examples of suitable synthetic polymers include polyethylene glycol (PEG), polyvinylpyrrolidone (PVP), polyvinyl alcohol (PVA), various Pluronics (poloxamers), or carbomers (e.g., Carbomer 940 or Carbomer 934). Examples of suitable modified celluloses include methylcellulose, carboxymethylcellulose (CMC), hydroxyethylcellulose (HEC), hydroxymethyl cellulose (HMC), hydroxypropyl cellulose (HPC), hydroxypropyl-methylcellulose (HPMC), or other cellulose-based gelling agents.

A variety of gelling agents are commercially available and can be obtained in many suitable molecular weights and ranges. For example, the molecular weights of the gelling agent can be about 1 kDa to about 1,000 kDa, about 10 kDa to about 1,000 kDa, about 100 kDa to about 1,000 kDa, or about 50 kDa to about 500 kDa. Examples of thickening agents include lanolin, hard paraffin, liquid paraffin, white petrolatum, soft yellow paraffin or soft white paraffin, white beeswax, yellow beeswax, propolis (propoleum), cetostearyl alcohol, cetyl alcohol, dimethicones, emulsifying waxes, microcrystalline wax, oleyl alcohol and stearyl alcohol. A gelling agent or thickening agent can be present in a formulation at about 0.05 wt. % to about 20 wt. %, typically about 0.1 wt. % to about 10 wt. %, about 0.1 wt. % to about 5 wt. %, about 0.5 wt. % to about 2 wt. %, about 0.8 wt. % to about 2 wt. %, or about 1-1.5 wt. %. In one embodiment, the composition comprises 0.5 wt. % to 15 wt. %, 1 wt. % to 10 wt. %, or 2 wt. % to 10 wt. %, of one or more thickening agents or gelling agents.

One or more gelling agents or thickening agents may be included in a single formulation. Such agents can be employed with liquid carriers to form spreadable gels, pastes, ointments, soaps, and the like, for application directly to the skin of the user.

### pH Adjusting Agents

Topical formulations of the present invention can also comprise a pH adjusting agent. In one embodiment, the pH adjusting agent is a base. Suitable pH adjusting bases include bicarbonates, carbonates and hydroxides such as alkali or alkaline earth metal hydroxide as well as transition metal hydroxides. In another embodiment, the pH adjusting agent is an acid, an acid salt, or mixtures thereof. In a further embodiment, the pH adjusting agent is a buffer. Suitable buffers include citrate/citric acid buffers, acetate/acetic acid buffers, phosphate/phosphoric acid buffers, formate/formic acid buffers, propionate/propionic acid buffers, lactate/lactic acid buffers, carbonate/carbonic acid buffers, ammonium/ammonia buffers, and the like.

### Solutions and Dispersions

Solutions of an active or its salts can be prepared in water, optionally mixed with a nontoxic surfactant. Dispersions can be prepared in glycerol, liquid polyethylene glycols, triacetin, or in a pharmaceutically acceptable oil, or mixtures thereof. Under ordinary conditions of storage and use, preparations may contain a preservative to prevent the growth of microorganisms.

Pharmaceutical dosage forms can include sterile aqueous solutions or dispersions comprising the active ingredient adapted for the extemporaneous preparation of sterile solutions or dispersions, optionally encapsulated in liposomes. The ultimate dosage form should be fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity of the composition can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the compositions can be brought about by agents delaying absorption, for example, aluminum monostearate and/or gelatin.

Solutions can be prepared by incorporating the active in a desired amount in the appropriate solvent or oil with various other ingredients enumerated herein, as desired, followed by optional filter sterilization. For powders used in the preparation of solutions, methods of preparation can include vacuum drying and freeze drying techniques, which yield a powder of the active plus any additional desired ingredient present in the prepared solutions.

### Gels

Gels are clear, sticky, jelly-like semisolids or solids prepared from high molecular weight polymers in an aqueous or alcoholic base. Alcoholic gels are often drying and cooling. Non-alcoholic gels are more lubricating. Gels or jellies can be produced using a suitable gelling agent including, but not limited to, gelatin, tragacanth, a carbomer, or a cellulose derivative and may include glycerol as a humectant, an emollient, and/or a preservative. In some embodiments, gel formulations will include the same or similar ingredients as a solution or dispersion, with the addition of a gelling agent.

The gel can include a nonionic copolymer gelling agent. In one embodiment, the gelling agent is a nonionic polyoxyethylene-polyoxypropylene copolymer gel, for example, a Pluronic gel such as Pluronic F-127 (BASF Corp.), to provide a pluronic gel-based formulation. This gel can be advantageous because it is a liquid at low temperatures but rapidly sets at physiological temperatures, which confines the release of the agent to the site of application or immediately adjacent that site. Other formulations can be carboxymethylcellulose (CMC)-based formulations, hydroxymethyl cellulose (HMC)-based formulations, hydroxypropyl cellulose (HPC)-based formulations, or hydroxypropylmethylcellulose (HPMC)-based formulations, and the like.

### Creams

Creams are viscous liquids or semisolid emulsions, either oil-in-water or water-in-oil. Cream bases are water-washable, and comprise an oil phase, an emulsifier, and an aqueous-phase. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant (a substance, such as glycerin, sorbitol, or urea, that absorbs or helps another substance retain moisture).

The emulsifier in a cream formulation is generally a nonionic, anionic, cationic, or amphoteric surfactant. Examples of emulsifiers include, but are not limited to, fatty alcohol polyoxyethylene ether (Peregal A-20), stearates such as polyoxylstearate (Softener SG), glyceryl stearate and pegylated forms of glyceryl stearate such as PEG-5 glyceryl stearate, cetyl alcohol, dithranol, or a combination thereof. Oil-phase ingredients can include, but are not limited to, dimethicone, dimethiconol, cyclomethicone, diisopropyl adipate, cetyl alcohol, stearyl alcohol, paraffin, petrolatum, almond oil, stearic acid, or a combination thereof. In particular aspects, aqueous ingredients can include, but are not limited to, purified water, glycerol (glycerin), propylene glycol, ethyl paraben, a humectant, or a combination thereof.

### Ointments

Ointments are semisolid preparations that include the active incorporated into a fatty, waxy, or synthetic base. Ointments are typically based on petrolatum or other petroleum derivatives. The specific ointment base to be used, as will be appreciated by those skilled in the art, is one that will provide for suitable drug delivery and other desired characteristics such as emolliency or the like. As with other carriers or vehicles, an ointment base is typically inert, stable, non-irritating and non-sensitizing.

Ointment bases may be generally grouped in four classes: oleaginous bases; emulsifiable bases; emulsion bases; and water-soluble bases. Oleaginous ointment bases can include, for example, vegetable oils, fats obtained from animals, and semisolid hydrocarbons obtained from petroleum. Emulsifiable ointment bases, also known as absorbent ointment bases, contain little or no water and can include, for example, hydroxystearin sulfate, anhydrous lanolin, and hydrophilic petrolatum. Emulsion ointment bases are either water-in-oil (W/O) emulsions or oil-in-water (O/W) emulsions, and the oil components can include, for example, cetyl alcohol, glyceryl monostearate, lanolin, and stearic acid. Water-soluble ointment bases can be prepared from polyethylene glycols of varying molecular weight.

### Lotions

Lotions are liquid or semiliquid preparations in which solid particles, including the active agent(s), are present in a water or alcohol base. Lotions are usually suspensions of solids, and can include a liquid oily emulsion of the oil-in-water type. Lotions are often desirable formulations because of the ease of applying a more fluid composition. It is generally advantageous for the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

### Pastes

Pastes are semisolid dosage forms in which the active agent is suspended in a suitable base. Depending on the nature of the base, pastes are divided between fatty pastes or those made from a single-phase aqueous gel. The base in a fatty paste is generally petrolatum, hydrophilic petrolatum, or the like. The pastes made from single-phase aqueous gels generally incorporate carboxymethylcellulose or the like as a base.

### Foams

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Accordingly, the composition described herein may be formulated for any desired form of topical or transdermal administration, including slow or delayed release preparations. Formulations may include known antioxidants (e.g., vitamin E); buffering agents; lubricants (e.g., synthetic or natural beeswax); sunscreens (e.g., para-aminobenzoic acid); and cosmetic agents (e.g., coloring agents, fragrances, essential oils, moisturizers, or drying agents).

An auxiliary agent such as casein, gelatin, albumin, or sodium alginate may also be included in various formulations. Adjuvants such as fragrances and additional antimicrobial agents can be added to optimize the properties for a given use. Examples of fragrances include Ylang-Ylang oil, lavender oil, powder scent, jasmine, gardenia oil, or green tea oil. In addition, substances such as wetting or emulsifying agents, stabilizing agents, or pH buffering agents, may also be included. When a water-based carrier is used, the composition is typically near a neutral pH(+/−about 1, or 2, pH units).

Further examples of dermatological ingredients and compositions for delivering active agents to the skin are known to the art. Such dermatological compositions can be used in combinations with the actives described herein in place of other actives.

The compositions described above can be prepared using standard compounding techniques. For example, for a composition that includes an active, or a salt of the active it can be triturated to reduce particle size. A second active can then be added with a small amount of carrier such as polysorbate 80 and/or ethoxy diglycol to wet the actives. This mixture can then be incorporated into a desired amount of oil using principles of geometric dilution until a smooth and uniform suspension is formed. This suspension can then be combined with other ingredients, such as a fragrance, to provide a therapeutic composition. The suspension can also be combined with other ingredients to form a variety of formulations, such as a gel, a jelly, a cream, an ointment, a wax, a lotion, a paste, a foam, or an aerosol. The suspension, or a gel, jelly, cream, ointment, wax, lotion, or paste can also be incorporated into a patch, such as an occlusive patch, to further improve transdermal penetration.

### Nanoparticles

Nanoparticles may be composed of materials such as polymers, lipids (e.g., liposomes) or dendrimers, such as PAMAM, PEI, PPI, or polylysine containing dendrimers. For example, the use of a nanoemulsion containing nanoparticles comprising the active, provides for different physical and chemical properties, for instance, nanoparticles may allow for deeper penetration into the skin, delivering treatment to more layers of skin and beyond. The nanoparticles may be coated with and/or embedded with (encapsulate) the composition. The nanoparticles may be formed using techniques such as electrospraying. The nanoparticles may be formed by grinding particles comprising the active to a desired size. The nanoparticles may include a surface coating.

The nanoparticles may be from about 1 nm to about 100 nm, about 100 nm to about 2,500 nm, or about 2,500 nm to about 10,000 nm in diameter. Nanoparticles may be formed of polymers or surfactants. Polymers and surfactants include but are not limited to non-ionic surfactants, anionic surfactants, e.g., carboxylates such as alkyl carboxylates-fatty acid salts or carboxylate fluoro surfactants; sulfates such as alkyl sulfates (e.g., sodium lauryl sulfate) or alkyl ether sulfates (e.g., sodium laureth sulfate); sulfonates, e.g., docusates (e.g., dioctyl sodium sulfosuccinate) or alkyl benzene sulfonates; and phosphate esters such as alkyl aryl ether phosphates or alkyl ether phosphates; cationic surfactants including fatty amine salts and quaternary ammoniums; zitterionic surfactants such as those with a quaternary amine group and a sulfonic or carboxyl group, natural polymers and synthetic polymers including but not limited to alginate, hyaluronate, chitosan, gelatin, cellulose, e.g., ethylcellulose, PVP, PEG or acylates. In one embodiment, the surfactant includes polyoxyethylene, poloxamer, poloxamine, or polysorbate.

The compounds or pharmaceutically acceptable derivatives thereof may be formulated as aerosols for topical application, such as by inhalation. These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye or in the mouth, in the form of gels, creams, and lotions and for application to the eye or for intracisternal or intraspinal application. Topical administration is contemplated for dermal or transdermal delivery and for administration to the eyes or mucosa, including buccal mucosa, or for inhalation therapies. Nasal, buccal, lingual and sublingual formulations of the active compound alone or in combination with other pharmaceutically acceptable excipients enumerated herein can also be administered.

These solutions, particularly those intended for ophthalmic use, may be formulated as 0.01%-10% (vol %) isotonic solutions, pH about 5-7, with appropriate salts.

### Compositions for Other Routes of Administration

Other routes of administration, such as transdermal patches, including iontophoretic and electrophoretic devices, vaginal and rectal administration, are also contemplated herein.

Transdermal patches, including iontophoretic and electrophoretic devices, are well known to those of skill in the art. For example, pharmaceutical dosage forms for rectal administration are rectal suppositories, capsules and tablets for systemic effect. Rectal suppositories as used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by moulding. The weight of a rectal suppository, in one embodiment, is about 2 to 3 gm.

Tablets and capsules for rectal administration are manufactured using the same pharmaceutically acceptable substance and by the same methods as for formulations for oral administration.

Other transdermal delivery methods known in the art or as described herein, including, for example, methods directed to 1) the use of chemical penetration enhancers or skin enhancers; 2) liposome-mediated delivery; 3) electroporation; 5) sonophoresis (ultrasound waves); 6) mechanical (e.g., microporation) devices, and/or 7) air pressure.

Methods suitable for transdermal delivery of the agents described herein can include, for example, methods directed to enhancing the transport of material across the skin pores by increasing the rate of transport across existing pores or by amplifying the number of available skin pores through the creation of artificial pores.

Transdermal delivery can be carried out by the use of chemical or penetration enhancers, including for example, a pharmaceutically acceptable oil of vegetable, nut, ethoxylated oil, PEG, linoleic acid, ethanol, methanol, and/or agents which delipidize the stratum comeum. Suitable oils include meadowfoam oil, castor oil, jojoba oil, corn oil, sunflower oil, sesame oil, all of which may be optionally ethoxylated. In addition, transdermal patches can be used for the topical or transdermal delivery of a composition described herein. A patch can also be adapted for delivery of dry or lyophilized forms of the compositions described herein. Other patch technology that can be employed in conjunction with the compositions described herein, e.g., in a reservoir of the patch. Transdermal delivery can also be carried out by liposome-mediated delivery methods (e.g., delivery facilitated by application of lipophilic membrane compositions). Transdermal delivery systems can also be employed in conjunction with a wide variety of iontophoresis or electrotransport systems. When a sonophoresis technique is used, one ultrasonic frequency can be applied to the skin, or two or more different ultrasonic frequencies can be applied to the skin (e.g., one low and one high ultrasonic frequency). As with the other techniques described above, this technique can be used in combination with other techniques, such as prior to the topical application of a composition described herein, including the application of a transdermal patch.

Another transdermal drug delivery technique that can be used in conjunction with the compositions described herein includes employing a device to use air pressure to inject a small stream of the composition through the top layers of the skin without the aid of a needle. The air pressure gun can be the same as or similar to the devise used to provide vaccines to children. Small, disposable pen-like devices are also suitable, as for diabetics who take insulin daily.

### Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions.

Liposomal suspensions, including tissue-targeted liposomes, such as tumour-targeted liposomes, may also be suitable as pharmaceutically acceptable carriers. These may be prepared according to methods known to those skilled in the art. For example, liposome formulations may be prepared. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Co-Administration with Other Drugs

In accordance with another aspect of the present invention, it is contemplated that the compound as described herein may be administered to a subject in need thereof in combination with medication considered by those of skill in the art to be current standard of care for the condition of interest. Such combinations provide one or more advantages to the subject, e.g., requiring reduced dosages to achieve similar benefit, obtaining the desired therapeutic effect in less time, and the like.

Compounds in accordance with the present invention may be administered as part of a therapeutic regimen with other drugs. It may be desirable to administer a combination of active compounds, for example, for the purpose of treating a particular disease or condition. Accordingly, it is within the scope of the present invention that two or more pharmaceutical compositions, at least one of which contains a compound according to the present invention, may be combined in the form of a kit suitable for co-administration of the compositions.

In one embodiment, of the methods of the present inventions a compound of the present invention may be administered with a second therapeutic agent. In one embodiment the second therapeutic agent may be selected from one or more of the following categories:
(i) Anti-cancer agents such as cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine, gemcitabine, 5-fluorouracil, tegafur, raltitrexed, methotrexate, pemetrexed, leucovorin, cytosine arabinoside, floxuridine, cytarabine, 6-mercaptopurine, 6-thioguanine, fludarabine phosphate, pentostatine, hydroxyurea, trifluridine, trifluracil, adriamycin, bleomycin, doxorubicin, daunomycin, epirubicin, idarubicin, mitomycin-C, dactinomycin, mithramycin, vincristine, vinblastine, vindesine and vinorelbine, taxol, taxotere, eribulin, carfilzomib, bortezomib, etoposide, teniposide, amsacrine, topotecan, irinotecan, mitoxantrone, camptothecin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, IFN-alpha, azacitidine, decitabine or the orally bioavailable cedazuridine combination product thereof, vorinostat, MS-275, panobinostat, romidepsin, valproic acid, mocetinostat (MGCD0103), pracinostat SB939, belinostat, panobinostat, irabectedin, tamoxifen, fulvestrant, toremifene, raloxifene, droloxifene, iodoxyfene, bicalutamide, flutamide, nilutamide, cyproterone acetate, goserelin, leuprorelin, buserelin, progestogens, megestrol acetate, anastrozole, letrozole, vorazole, exemestane, finasteride, navelbene, CPT-II, anastrazole, letrazole, capecitabine, cyclophosphamide, ifosamide, and droloxafine; and abiraterone, enzalutamide, lanreotide, dasatinib, bosutinib (SKI-606), trastuzumab, pertuzumab, panitumumab, cetuximab, gefitinib, erlotinib, 6-acrylamido-/V-(3-chloro-4-fluorophenyl)-7-(3-morpholinopropoxy)-quinazolin-4-amine (CI 1033), afatinib, vandetanib, osimertinib, rociletinib, lapatinib, CTLA-4, 4-IBB, PD-I, imatinib, nilotinib (AMN107), sorafenib, tipifarnib and lonafarnib, vemurafenib, dabrafenib, trametinib, cobimetinib, ponatinib, palbociclib, everolimus, ruxolitinib, ibrutinib, ceritinib, crizotinib, ectinib, cabozantirsib, vismodegib, soni iegib, BAL3833, regorafenib, vandetanib, vatalanib, sunitinib, axitinib, pazopanib, lenvatinib, talimogene laherparepvec, denosumab, obinuluzumab, biinatomumab, dinutuximab, idarucizumab, daratumumab, necitumumab, elotuzumab, olaratumab, alemtuzumab, rituximab, ibritumomab tiuxetan, ofatumumab, peginterferon alpha-2b, aldesleukin, Gardasil, Cervarix, Oncophage, Sipuleucel-T (Provenge) gp100, Ad.p53 DC, pembrolizumab, atezolizumab, indoximod, MK-3475, nivolumab, MEDI-4736, RG-7446, ipilumumab, brentuximab vedotin, irastuzumab emtansine, fludaribine (fludara), cladribine, pentostatin, idelalisib, perifosine, birinapant, LCL161, AEG40730, SM-164, LBW242, ML101, AT-406, GDC-0917, AEG35156, HGS1029, bortezomib, ixazomib, carfilzomib, marizomib (NPI-0052), MLN9708, olaparib, rucaparib; antiapoptotic, venetoclax, navitoclax, chimeric antigen receptors.
(ii) Anti-inflammatory agents such as meloxicam, feoprofen, oxaprozin, salsalate, etoricoxib, tenoxicam, aspirin, nabumetone, flurbiprofen, mefenamic acid, phenylbutazone, lornoxicam, indomethacin, etodolac, diflunisal, ketoprofen, valdecoxib, tolfenamic acid, piroxicam, sulindac, tolmetin, ketorolac, loxoprofen, acetaminophen, bromfenac, diclofenac, ibuprofen, meclofenamate, nabumetone, naproxen, nepafenac, celecoxib, triamcinolone acetonide, colchicine, hydrocortisone, hydrocortisone acetate, methylprednisolone, aclomethasone dipropionate, emricasan, PXS-4728 (BI 1467335), namodenoson, roflumilast, crisaborole, PF-07038124, etrasimod, ritlecitinib, ruxolitinib, pacritinib, delgocitinib, tofacitinib, erdulatinib, brepocitinib, momelotinib, PF-07295324, PF-07259955.
(iii) Anti-hypertensive agent such as hydrochlorothiazide, chlorthalidone, furosemide, spironolactone, triamterene, amiloride, benazepril, captopril, lisinopril, enalapril, ramipril, fosinopril, moexipril, perindopril, quinapril, trandolapril, losartan, candesartan, valsartan, telmisartan, clonidine, methyldopa, propranolol, nadolol, timolol, pindolol, labetolol, metoprolol, atenolol, esmolol, betaxolol, carvedilol, prazosin, terazosin, doxazosin, phenoxybenzamine, phentolamine, verapamil, diltiazem, nifedipine, felodipine, amlodipine, nimodipine, diazoxide, minoxidil, pinacidil, nicorandil, hydralazine, sodium nitroprusside.
(iv) Anti-fibrotic agent such as pirfenidone, nintedanib, cenicriviroc, selonsertib, lanifibranor, nimacimab, nitrazoxanide, NGM282, apararenone, tipelukast, Actimmune, ponatinib, lenvatinib, dovitinib, lucitanib, danusertinib, brivatinib, erdafitinib, bexotegrast, PD173074, PD166866, AZD4547, BGJ398, LY2874455, TAS-120, ARQ087, BLU9931, FGF401, BAY-1163877, ENMD-2076, IMCA1, FGF401, DEB101347, FIIN-2, GP-369, PRO-001, H3B-6527, BAY1187982, MFGR1877S, FP-1039, BLU554, PRN1371, S49076, SU6668, SU5416, TERN-501, MGL-3196, VK2809.
(v) Anti-angiogenesis agent such as axitinib, bevacizumab, cabozantinib, everolimus, lenalidomide, lenvatinib, pazopanib, ramucirumab, regorafenib, vandetanib, vatalanib, sunitinib, ziv-aflibercept, thalidomide, pomalidomide.
(vi) Anti-diabetic agent such as gliclazide, glimepiride, repaglinide, metformine, pioglitazone, rosiglitazone, acarbose, lingliptine, saxagliptine, sitagliptine, alogliptine, exenatide, liraglutide, dulaglutide, lixisenatide, semaglutide, canaglifozine, dapagliflozine, empagliflozine, ertugliflozine.
(vii) Immunosuppressive agent such as prednisone, budesonide, prednisolone, tofacitinib, cyclosporine, tacrolimus, sirolimus, everolimus, azathioprine, leflunomide, mycophenolate, abatacept, adalimumab, anakinra, certolizumab, etanercept, golimumab, infliximab, ixekizumab, natalizumab, rituximab, secukinumab, tocilizumab, ustekinumab, vedolizumab, basiliximab, daclizumab.
(viii) Anti-bacterial agent such as daptomycin, delafloxacin, telavancin, ceftaroline, fidaxomicin, amoxicillin, ampicillin, becampicillin, carbenicillin, cloxacillin, dicloxacillin, flucloxacillin, mezlocillin, nafcillin, oxacillin, penicillin G, penicillin V, piperacillin, pivampicillin, pivmecillinam, ticarcillin, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrzole, cefivitril, cefmatilen, cefmepidium, cefovecin, cefoxazole, cefrotil, cefsumide, cefuracetime, ceftioxide, ceftazidime, avibactam, ceftolozane, tazobactam, aztreonam, vaborbactam, imipenem, doripenem, ertapenem, meropenem, azithromycin, erythromycin, clarithromycin, dirithromycin, roxithromycin, telithromycin, clindamycin, lincomycin, pristinamycin, quinupristin, dalfopristin, amikacin, gentamicin, kanamycin, neomycin, netilmicin, paromomycin, streptomycin, tobramycin, flumequine, nalidixic acid, oxolinic acid, piromidic acid, pipemidic acid, rosoxacin, ciprofloxacin, enoxacin, lomefloxacin, nadifloxacin, norfloxacin, ofloxacin, perfloxacin, rufloxacin, balofloxacin, gatifloxacin, grepafloxacin, levofloxacin, moxifloxacin, pazufloxacin, sparfloxacin, temafloxacin, tosufloxacin, besifloxacin, delafloxacin, clinafloxacin, gemifloxacin, prulifloxacin, sitafloxacin, trovafloxacin, sulfamethizole, sulfamethoxazole, sulfisoxazole, trimethoprim, demeclocycline, doxycycline, minocycline, oxytetracycline, tetracycline, tigecycline, chloramphenicol, metronidazole, tinidazole, nitrofurantoin, vancomycin, teicoplanin, telavancin, linezolid, cycloserine, rifampin, rifabutin, rifapentine, rifalazil, bacitracin, polymixin B, viomycin, capreomycin.
(ix) Anti-fungal agent such as amorolfin, butenafine, naftifine, terbinafine, bifonazole, butoconazole, clotrimazole, econazole, fenticonazole, ketoconazole, isoconazole, luliconazole, miconazole, omoconazole, oxiconazolesertaconazole, sulconazole, tioconazole, terconazole, albaconazole, efinaconazole, fluconazole, isavuconazole, itraconazole, posaconazole, ravuconazole, voriconazole, abafungin, amphotericin B, nystatin, natamycin, trichomycin, anidulafungin, caspofungin, micafungin, tolnaftate, flucytosine, butenafine, griseofulvin, ciclopirox, selenium sulfide, tavaborole.
(x) Anti-pruritic agent such as doxepin, tacrolimus, pimecrolimus, menthol, capsaicin, salicylic acid, pramoxine, lidocaine, polidocanol, N-palmitoylethanolamine, prednisone, prednisolone, mirtazapine, paroxetine, fluvoxamine, sertraline, naltrexone, methylnaltrexone, butorphanol, nalfurafine, gabapentin, pregablin, aprepitant, loratadine, desloratadine, cetirizine, levocetirizine, NGX-4010, TS-022.
(xi) Metabolic agent such as obeticholic acid, elafibranor, aramchol, seladelpar, MGL-3196, tropifexor, MSDC-0602K, BMS-986036, semaglutide, EDP-305, gemcabene, PF-05221304, PF-06865571, PF-06835919, LIK066, LMB763, vitamin E, acarbose, miglitol, pramlintide, alogliptan, linagliptan, saxagliptin, sitagliptin, albiglutide, dulaglutide, exenatide, liraglutide, lixisenatide, insulin, nateglinide, repaglinide, metformin, canagliflozin, dapagliflozin, empagliflozin, chlorpropamide, glimepiride, glipizide, glyburide, tolazamide, tolbutamide, rosiglitazone, pioglitazone, atorvastatin, amlodipine, simvastatin, ezetimibe, lovastatin, sitagliptin, cholestyramine, colesevelam, colestipol, fenofibrate, gemfibrozil, fenofibric acid, niacin, icosapent, mipomersen, lomitapide, evolocumab, alirocumab, fluvastatin, pravastatin, rosuvastatin, pitavastatin, simvastatin, cerivastatin, allopurinol, lesinurad, pegloticase, febuxostat, rasburicase, ivacaftor, velaglucerase alfa, imiglucerase, alglucosidase alfa, laronidase, cerliponase alfa, alglucerase, idursulfase, taliglucerase alfa, agalsidase beta, sebelipase alfa, vestronidase alfa, galsulfase, elosulfase alfa, eliglustat, burosumab, migalastat, sapropterin, metreleptin, nitisinone, pegvaliase, asfotase alfa, inotersen, miglustat, orlistat, sodium phenylbutyrate, glycerol phenylbutyrate.

In one embodiment, a compound of the present invention may be administered in combination with other therapeutic treatments. For example, a compound of the present invention may be administered in combination with radiotherapy or chemotherapy. In one embodiment a compound of the present invention may be administered in combination with one or more additional anti-tumour agent and/or radiotherapy for the treatment of a cancer. In one embodiment a compound of the present invention may be administered in combination with wound covers and wound dressings. In one embodiment a compound of the present invention may be administered in combination with scar covers and scar dressings.

When two or more active ingredients are co-administered, the active ingredients may be administered simultaneously, sequentially or separately. In one embodiment a compound of the invention is co-administered simultaneously with a second therapeutic agent. In another embodiment a compound of the invention and the second therapeutic agent are administered sequentially. In a further embodiment a compound of the invention and the second therapeutic agent are administered separately.

The invention will now be described in greater detail, by way of illustration only, with reference to the following non-limiting examples. The examples are intended to serve to illustrate the invention and should not be construed as limiting the generality of the disclosure of the description throughout this specification.

### EXAMPLES

For purposes of this specification, the following abbreviations have the indicated meanings:
rt=room temperature
t=time
min=minute(s)
°C=degrees Celsius
h=hour(s)
RT=retention time
aq=aqueous
sat=saturated
NaHMDS=sodium hexamethyldisilazide
THF=tetrahydrofuran
DMF=dimethylformamide
TFA=trifluoroacetic acid
DIBAL=diisobutylaluminumhydride
MTBE=methyl tert-butylether
DIPEA=N,N-diisopropylethylamine
HATU=1-[bis(dimethylamino)methylene]-1H-1,2,3-triazolo[4,5-b]pyridinium 3-oxid hexafluorophosphate
LC-MS or LCMS=liquid chromatography-mass spectroscopy
rpm=revolutions per minute
NMR=nuclear magnetic resonance
DMSO=dimethylsulfoxide
Boc=tert-butyloxycarbonyl
HPLC=high-performance liquid chromatography (also, high pressure liquid chromatography)
TLC=thin-layer chromatography
V=volume(s)
conc=concentrated
RP=reverse-phase (chromatography)
NP=normal-phase (chromatography)

### Experimental: General Methods

All commercially available solvents and reagents were used as received. Where appropriate, reactions were carried out under an argon atmosphere. Reactions were monitored by either analytical thin-layer chromatography (TLC) or by analytical liquid chromatography-mass spectrometry (LC-MS) recorded on either a Shimadzu LC-MS 2020 instrument or an Agilent LC/MSD 1200 instrument using reverse-phase conditions. Purification of intermediates and final compounds was conducted, where necessary, using column chromatography or preparative HPLC. Normal-phase column chromatography was conducted under medium pressure either on silica gel or on prepacked silica gel cartridges using a flash chromatography system (CombiFlash Rf200, Teledyne Isco systems, USA). Reverse-phase column chromatography was conducted under low pressure on prepacked C18 cartridges using a flash chromatography system (Reveleris® X2). Eluents were monitored by UV light (λ=254/280 nm). ¹H-NMR spectra were recorded using either a Bruker 300 MHz NMR spectrometer or a Bruker Fourier 400/300 MHz spectrometer. Chemical shifts (δ) are reported as parts per million (ppm) relative to tetramethylsilane (TMS; internal standard). The following abbreviations are used for multiplicities: s=singlet; br s=broad singlet; d=doublet; t=triplet; q=quartet; m=multiplet; and br m=broad multiplet. Low resolution mass spectra (MS) were obtained as electrospray—atmospheric pressure ionization (ES-API) mass spectra, which were recorded on either a Shimadzu LCMS 2020 instrument or an Agilent LC/MSD 1200 instrument using reverse-phase conditions. All animal experiments performed were conducted in compliance with institutional guidelines and approval from local ethics committees.

Unless otherwise indicated, conventional methods of mass spectroscopy, NMR, HPLC and protein chemistry, biochemistry techniques are employed. Unless stated otherwise, starting materials for chemical synthesis and biological applications are available from commercial sources.

LC-MS method used for analysis is described here:

Method A: Shimadzu LC-MS 2020 instrument; LC-MS column Restek Raptor ARC-18 5 um (4.6×150 mm); mobile phase A: 0.1% formic acid in Milli Q Water, Mobile phase B: 0.1% formic acid in methanol (Gradient grade); Flow rate: 1.5 mL/min; Column oven temperature: 40 °C; Run time: 9.4 min; Injection volume: 5 μL; Binary Gradient flow program: Starting at 95% water (0.1% formic acid) and 5% methanol (0.1% formic acid) to 50% water (0.1% formic acid) and 50% methanol (0.1% formic acid) in 1.40 min, changing to 5% water (0.1% formic acid) and 95% methanol (0.1% formic acid) in 6.00 min and holding the condition until 7.40 min, back to water (0.1% formic acid) 95% and methanol (0.1% formic acid) 5% from 7.41 min to 9.40 min.

Method B: Quadruple Mass Spectrometer on Agilent LC/MSD 1200 Series (Column: ODS 2000 (50×4.6 mm, 5 m) operating in ES (+) or (−) ionization mode; T=30 °C; flow rate=1.5 mL/min; detected wavelength: 214 nm.

Experimental details regarding the synthesis of intermediate-1 (Int-1) have been disclosed in patents WO 2021/258159 and WO 2018/158140. The contents of which are incorporated by reference.

### Example 1

### Preparation of (E)-3-fluoro-2-((phenylsulfonyl)methyl)prop-2-en-1-amine hydrochloride (gram scale process; Compound 1)

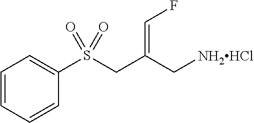

### Procedure A: Preparation of tert-butyl (E)-(3-fluoro-2-((phenylsulfonyl)methyl)allyl)carbamate

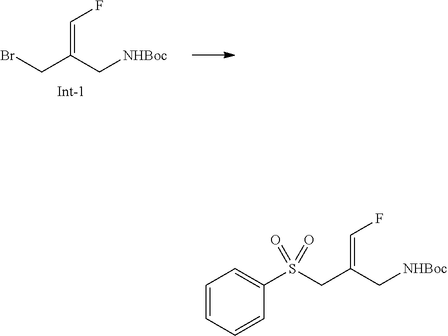 [SMILES: CC(C)(C)OC(=O)NC/C(=C\F)CBr ; CC(C)(C)OC(=O)NC/C(=C\F)CS(=O)(=O)c1ccccc1]

To a stirred solution of Int-1 (2.50 g, 9.32 mmol) in DMF (12 mL) was added sodium benzenesulfinate (2.30 g, 14.0 mmol) in one lot. The resulting mixture was stirred at rt for 2 h. TLC analysis after this time showed an absence of Int-1. Water (100 mL) was added and the reaction mixture was stirred at rt for 5 min. The precipitated solid was collected by filtration and the solid was washed with water (2×25 mL). The solid was dried in an oven at 60 °C overnight to afford tert-butyl (E)-(3-fluoro-2-((phenylsulfonyl)methyl)-allyl)carbamate (2.80 g, 91%). ¹H NMR (300 MHz, CDCl₃) δ ppm: 7.96-7.85 (m, 2H), 7.72 (td, J=7.2, 1.5 Hz, 1H), 7.62 (ddd, J=8.3, 6.4, 1.4 Hz, 2H), 6.39 (d, J=80.5 Hz, 1H), 5.03 (s, 1H), 3.98 (ddd, J=6.4, 2.8, 1.4 Hz, 2H), 3.75 (d, J=2.9 Hz, 2H), 1.46 (d, J=1.2 Hz, 9H).

### Procedure B: preparation (E)-3-fluoro-2-((phenylsulfonyl)methyl)prop-2-en-1-amine hydrochloride (Compound 1)

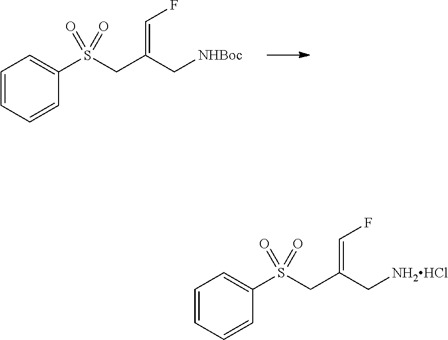

To a stirred suspension of tert-butyl (E)-(3-fluoro-2-((phenylsulfonyl)methyl)allyl)carbamate (2.80 g, 8.50 mmol) in MeOH (10 mL) was added HCl (4.0 M in 1,4-dioxane; 5.0 mL). The resulting mixture was stirred at rt for 2 h. TLC analysis after this time indicated complete consumption of starting material. The reaction mixture was concentrated in vacuo to remove volatiles. Ethyl acetate (25 mL) was added and the mixture was stirred for 5 min. The precipitated solid was collected by filtration and the solid was washed with ethyl acetate (25 mL). The solid was dried at 60 °C for 1 h affording Compound 1 (2.09 g, 93%).

¹H NMR (300 MHz, Methanol-d₄) δ ppm: 8.03-7.93 (m, 2H), 7.87-7.76 (m, 1H), 7.75-7.65 (m, 2H), 6.78 (d, J=80.5 Hz, 1H), 4.10 (d, J=2.8 Hz, 2H), 3.86 (d, J=2.5 Hz, 2H). MS: 230.1 [M+H]⁺; RT=2.85 min (Method A).

### Preparation of (E)-3-fluoro-2-((phenylsulfonyl)methyl)prop-2-en-1-amine hydrochloride (kilogram scale process; Compound 1)

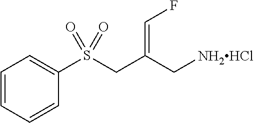

### Procedure C: Preparation of tert-butyl (E)-(3-fluoro-2-((phenylsulfonyl)methyl)allyl)-carbamate

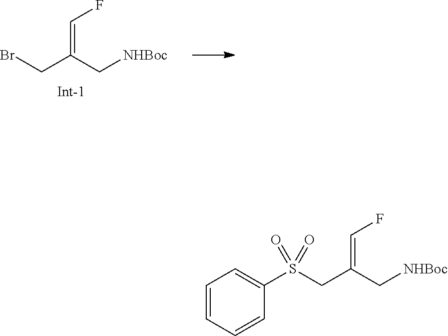 [SMILES: CC(C)(C)OC(=O)NC/C(=C\F)CBr ; CC(C)(C)OC(=O)NC/C(=C\F)CS(=O)(=O)c1ccccc1]

A reaction vessel was charged with Int-1 (8.4 kg, 1.0 eq), sodium benzenesulfinate (7.7 kg, 1.5 eq) and DMF (33.6 L, 4 V). The resulting mixture was stirred at rt for 20 h. Water (67 L, 8 V) was added into the reactor and stirring was continued for 1 h. The solid was collected by filtration and the filter cake was washed with water (25.2 L, 3 V). The crude solid was dried under vacuum to afford crude product (8.9 kg, 95.8% purity; residual Int-1: 82 ppm). The crude product was dissolved in ethyl acetate (3 V) at 60 °C. To this was added n-heptane (30 V) dropwise. The mixture was then cooled to 20 °C and the resulting solid was collected by filtration and dried under vacuum to afford tert-butyl (E)-(3-fluoro-2-((phenylsulfonyl)methyl)-allyl)carbamate (8.6 kg, residual Int-1: 32 ppm).

### Procedure D: Preparation (E)-3-fluoro-2-((phenylsulfonyl)methyl)prop-2-en-1-amine hydrochloride (Compound 1)

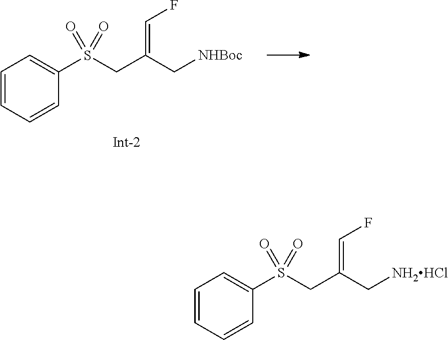

To a reaction vessel charged with tert-butyl (E)-(3-fluoro-2-((phenylsulfonyl)methyl)-allyl)carbamate (8.6 kg, 1.0 eq), and ethyl acetate (51.6 L, 6 V) under nitrogen was added HCl (4 M in ethyl acetate; 25.8 L, 3 V) dropwise. The resulting mixture was stirred at 25-30 °C for 16 h. The solid was collected by filtration and the filter cake was then washed with ethyl acetate (25.8 L, 3.0 V). The solid was then dried under vacuum at 45-50 °C to afford Compound 1 (6.3 kg).

### Preparation of (E)-3-((2-(aminomethyl)-3-fluoroallyl)sulfonyl)benzonitrile hydrochloride (Compound 2)

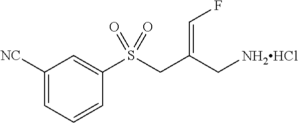

### Procedure E: Preparation of sodium 3-cyanobenzenesulfinate

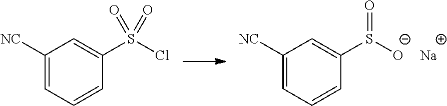 [SMILES: N#Cc1cccc(S(=O)(=O)Cl)c1 ; N#Cc1cccc(S(=O)[O-])c1 ; [Na+]]

To a stirring solution of sodium sulfite (945 mg, 7.50 mmol) and sodium carbonate (1.06 g, 10.0 mmol) in water (10 mL) at rt was added 3-cyanobenzenesulfonyl chloride (1.01 g, 5.00 mmol) portion-wise over 5 min. The mixture was stirred at rt for 4 h. The mixture was concentrated to approximately 5 mL. The reaction mixture was purified directly by RP chromatography (Reveleris® C18 40 g; eluent: solvent A=(12 mM aq HCl); solvent B=methanol; 100% A for 4.8 min followed by gradient 0-50% B over 4.2 min. Finally, held at 50% B for 1 min. Flow rate 32 mL/min.). The desired fractions were pooled and sat. aq. NaHCO₃ (2.0 mL) was added. The mixture was concentrated under vacuum to afford crude sodium 3-cyanobenzenesulfinate (700 mg, 74%) that was further used as such. ¹H NMR (300 MHz, Deuterium Oxide) 6 7.88 (td, J=1.7, 0.6 Hz, 1H), 7.81 (dt, J=7.8, 1.4 Hz, 1H), 7.78 (td, J=7.7, 1.5 Hz, 1H), 7.58 (td, J=7.8, 0.6 Hz, 1H).

### Procedure F: Preparation of tert-butyl (E)-(2-(((3-cyanophenyl)sulfonyl)methyl)-3-fluoroallyl)carbamate

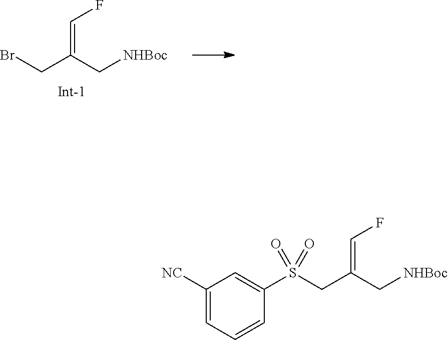 [SMILES: CC(C)(C)OC(=O)NC/C(=C\F)CBr ; CC(C)(C)OC(=O)NC/C(=C\F)CS(=O)(=O)c1cccc(C#N)c1]

To a stirring mixture of crude sodium 3-cyanobenzenesulfinate (284 mg, ca. 1.5 mmol) in DMF (2.5 mL) was added Int-1 (268 mg, 1.00 mmol). The resulting mixture was stirred at rt for 1 h. TLC analysis after this time indicated complete consumption of Int-1. Water (10 mL) was added and the product was extracted with ethyl acetate (3×10 mL). The combined organics were washed with brine, dried over Na₂SO₄ and then concentrated in vacuo. The crude material was purified by NP chromatography (CombiHash® Redisep column 12 g silica; eluent: solvent A=cyclohexane; solvent B=ethyl acetate; 10% B for 1 min followed by gradient 10%-60% B over 8 min. Finally, held at 60% B for 2 min. Flow rate 30 mL/min) to afford tert-butyl (E)-(2-(((3-cyanophenyl)sulfonyl)methyl)-3-fluoroallyl)carbamate (95 mg, 27%). ¹H NMR (300 MHz, CDCl₃) δ 8.24 (d, J=1.8 Hz, 1H), 8.16 (dt, J=7.9, 1.5 Hz, 1H), 7.99 (dt, J=7.8, 1.4 Hz, 1H), 7.82-7.73 (m, 1H), 6.49 (d, J=80.2 Hz, 1H), 4.97 (s, 1H), 4.04-3.91 (m, 2H), 3.81 (d, J=2.7 Hz, 2H), 1.46 (s, 9H).

### Procedure G: Preparation of (E)-3-((2-(aminomethyl)-3-fluoroallyl)sulfonyl)benzonitrile hydrochloride (Compound 2)

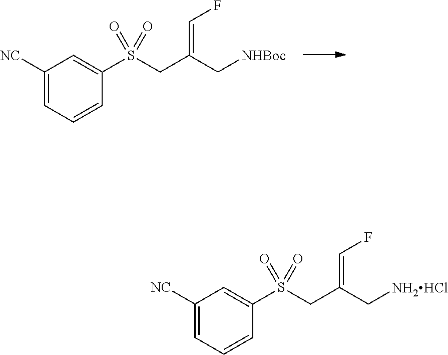

To a suspension of tert-butyl (E)-(2-(((3-cyanophenyl)sulfonyl)methyl)-3-fluoroallyl)carbamate (94.0 mg, 0.27 mmol) in 1,4-dioxane (2.0 mL) at rt was added HCl (4.0 M in 1,4-dioxane; 2.0 mL, 8.0 mmol). The resulting mixture was stirred at rt for 2 h. TLC analysis after this time showed complete consumption of starting material. The reaction mixture was concentrated under vacuum. Ethyl acetate (5 mL) was added and the mixture was stirred for 5 min. The precipitated product was then collected by filtration and the filter “cake” was washed with ethyl acetate (5 mL). The product was dried at 60 °C for 1 h to afford Compound 2 (55.0 mg, 71%). ¹H NMR (300 MHz, DMSO-d₆) δ 8.41 (brs, 4H), 8.29 (d, J=7.8 Hz, 1H), 8.22 (d, J=8.0 Hz, 1H), 7.91 (t, J=7.9 Hz, 1H), 6.81 (d, J=81.2 Hz, 1H), 4.66-4.31 (m, 2H), 3.70-3.48 (m, 2H). LC-MS: m/z 255 [M+H]+; RT=2.45 min (Method A).

### Procedure H: Preparation of tert-butyl (E)-(3-fluoro-2-(((3-(4,4,5,5-tetramethyl-1,3,2-dioxaborolan-2-yl)phenyl)sulfonyl)methyl)allyl)carbamate

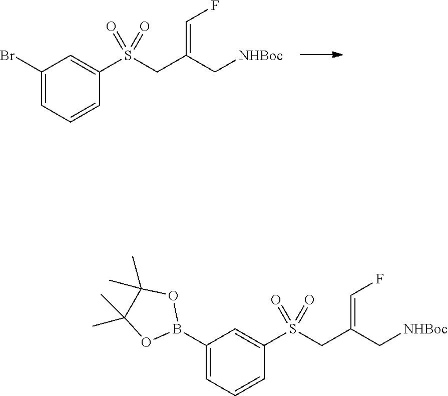 [SMILES: CC(C)(C)OC(=O)NC/C(=C\F)CS(=O)(=O)c1cccc(Br)c1 ; CC(C)(C)OC(=O)NC/C(=C\F)CS(=O)(=O)c1cccc(B2OC(C)(C)C(C)(C)O2)c1]

A stirring solution of tert-butyl (E)-(2-(((3-bromophenyl)sulfonyl)methyl)-3-fluoroallyl)carbamate (0.41 g, 1.0 mmol), bis(pinacolato)diboron (0.28 g, 1.1 mmol) and potassium acetate (0.29 g, 3.00 mmol) in 1,4-dioxane (10.0 mL) was purged with nitrogen for 5 min. To the solution was then added 1,1′-bis(diphenylphosphino)ferrocene-palladium(II)dichloride dichloromethane complex (41 mg, 0.05 mmol) and the resulting mixture was then heated at 80 °C overnight. The reaction mixture was then cooled to rt and diluted with water (25 mL). The product was extracted with ethyl acetate (3×25 mL). The combined organics were washed with brine, dried over Na₂SO₄ and then concentrated in vacuo to afford crude tert-butyl (E)-(3-fluoro-2-(((3-(4,4,5,5-tetramethyl-1,3,2-dioxaborolan-2-yl)phenyl)sulfonyl)methyl)-allyl)carbamate (0.46 g, 99%). The crude material was progressed to the next step immediately without purification.

### Procedure I: Preparation of tert-butyl (E)-(3-fluoro-2-(((3-(thiazol-2-yl)phenyl)sulfonyl)methyl)allyl)-carbamate

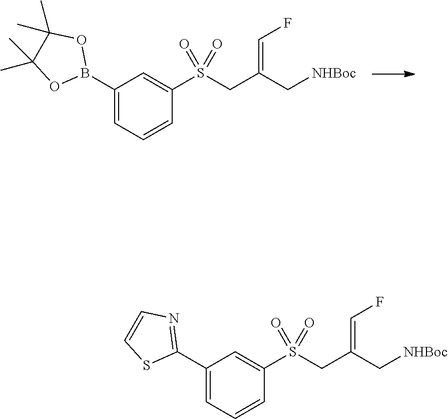 [SMILES: CC(C)(C)OC(=O)NC/C(=C\F)CS(=O)(=O)c1cccc(B2OC(C)(C)C(C)(C)O2)c1 ; CC(C)(C)OC(=O)NC/C(=C\F)CS(=O)(=O)c1cccc(-c2nccs2)c1]

A solution of 2-bromothiazole (164 mg, 1.00 mmol), tert-butyl (E)-(3-fluoro-2-(((3-(4,4,5,5-tetramethyl-1,3,2-dioxaborolan-2-yl)phenyl)sulfonyl)methyl)allyl)carbamate (228 mg, 0.50 mmol), and potassium phosphate tribasic (0.46 g, 2.00 mmol) in 1,4-dioxane (8.0 mL) and water (2.0 mL) was degassed by passing argon through it for 5 min. Tetrakis(triphenylphosphine)palladium(0) (58 mg, 0.05 mmol) was then added under argon and the reaction mixture heated at 90 °C for 5 h. TLC analysis after this time showed completion of the reaction. The reaction mixture was cooled to rt, diluted with ethyl acetate (50 mL) and then filtered through a plug of Celite™. The filtrate was then concentrated in vacuo. The crude material was purified by NP chromatography (CombiHash® Redisep column 12 g silica; eluent: solvent A=cyclohexane; solvent B=ethyl acetate; gradient 0%-40% B over 10 min. Finally, held at 40% B for 5 min. Flow rate 30 mL/min.) to afford tert-butyl (E)-(3-fluoro-2-(((3-(thiazol-2-yl)phenyl)sulfonyl)methyl)allyl)carbamate (120 mg, 51%). ¹H NMR (300 MHz, CDCl₃) δ 8.48 (t, J=1.8 Hz, 1H), 8.28 (ddd, J=7.8, 1.8, 1.1 Hz, 1H), 7.99-7.90 (m, 2H), 7.69 (t, J=7.8 Hz, 1H), 7.46 (d, J=3.3 Hz, 1H), 6.46 (d, J=80.5 Hz, 1H), 5.12 (s, 1H), 4.00 (ddd, J=6.4, 2.7, 1.3 Hz, 2H), 3.85-3.76 (m, 2H), 1.44 (s, 9H).

### Procedure J: Preparation of tert-butyl (E)-(3-fluoro-2-(((3-(2-methylthiazol-5-yl)phenyl)sulfonyl)-methyl)allyl)carbamate

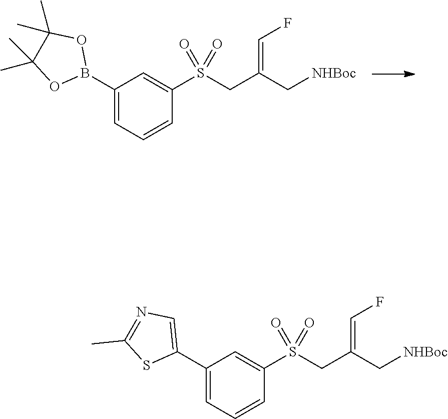 [SMILES: CC(C)(C)OC(=O)NC/C(=C\F)CS(=O)(=O)c1cccc(B2OC(C)(C)C(C)(C)O2)c1 ; Cc1ncc(-c2cccc(S(=O)(=O)C/C(=C/F)CNC(=O)OC(C)(C)C)c2)s1]

A solution of 5-bromo-2-methyl-thiazole (178 mg, 1.00 mmol), tert-butyl (E)-(3-fluoro-2-(((3-(4,4,5,5-tetramethyl-1,3,2-dioxaborolan-2-yl)phenyl)sulfonyl)methyl)allyl)carbamate (227 mg, 0.50 mmol), potassium phosphate tribasic (0.46 g, 2.0 mmol) in 1,4-dioxane (8 mL) and water (2 mL) was degassed by passing argon through it for 5 min. Tetrakis(triphenylphosphine)palladium(0) (58 mg, 0.05 mmol) was then added under argon and the reaction mixture heated at 90 °C for 5 h. TLC analysis after this time showed a trace of unreacted starting material. The reaction mixture was cooled to rt, diluted with ethyl acetate (50 mL) and then filtered through a plug of Celite®. The filtrate was concentrated in vacuo. The crude material was adsorbed on silica gel and purified by NP chromatography (CombiFlash® Redisep column 12 g silica; eluent: solvent A=cyclohexane; solvent B=ethyl acetate; 10% B for 1 min followed by gradient 10%-40% B over 10 min. Finally, held at 40% B for 7 min. Flow rate 30 mL/min.). LCMS analysis showed ~10% of an unidentified impurity. This material was therefore further purified by RP chromatography (Reveleris® C18 24 g; eluent: solvent A=12 mM aq HCl; solvent B=methanol; 20% B for 3 min followed by gradient 20-80% B over 15 min. Flow rate 32 mL/min.) to afford tert-butyl (E)-(3-fluoro-2-(((3-(2-methylthiazol-5-yl)phenyl)sulfonyl)methyl)allyl)-carbamate (75 mg, 31%). ¹H NMR (300 MHz, CDCl₃) δ 8.03 (d, J=1.9 Hz, 1H), 7.92 (s, 1H), 7.82 (dd, J=7.9, 1.8 Hz, 2H), 7.66-7.59 (m, 1H), 6.47 (d, J=80.5 Hz, 1H), 5.05 (s, 1H), 4.03-3.93 (m, 2H), 3.79 (d, J=2.7 Hz, 2H), 2.78 (s, 3H), 1.45 (s, 9H).

### Procedure K: Preparation of tert-butyl (E)-(3-fluoro-2-(((3-(3-methyl-1H-pyrazol-1-yl)phenyl)sulfonyl)methyl)allyl)carbamate

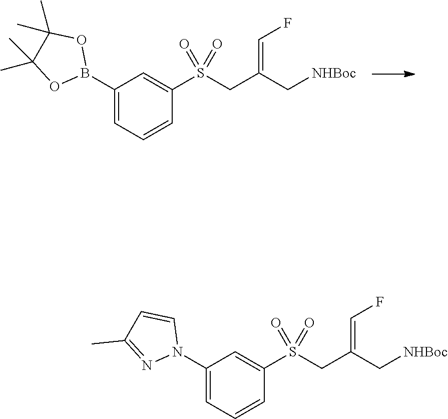 [SMILES: CC(C)(C)OC(=O)NC/C(=C\F)CS(=O)(=O)c1cccc(B2OC(C)(C)C(C)(C)O2)c1 ; Cc1ccn(-c2cccc(S(=O)(=O)C/C(=C/F)CNC(=O)OC(C)(C)C)c2)n1]

A mixture of 3-methyl-1H-pyrazole (123 mg, 1.50 mmol), cupric acetate (272 mg, 1.50 mmol) pyridine (0.16 mL, 2.00 mmol) and tert-butyl (E)-(3-fluoro-2-(((3-(4,4,5,5-tetramethyl-1,3,2-dioxaborolan-2-yl)phenyl)sulfonyl)methyl)-allyl)carbamate (455 mg, 1.00 mmol) in acetonitrile (10 mL) was stirred at rt overnight. The reaction mixture was concentrated under vacuum and the crude material was purified by NP chromatography (CombiFlash® Redisep column 24 g silica; eluent: solvent A=cyclohexane; solvent B=ethyl acetate; 10% B for 1 min. followed by gradient 10%-50% B over 9.5 min. Flow rate 35 mL/min.) to afford tert-butyl (E)-(3-fluoro-2-(((3-(3-methyl-1H-pyrazol-1-yl)phenyl)sulfonyl)methyl)allyl)carbamate (25 mg, 6%).

### Procedure L: Preparation of tert-butyl (E)-(3-fluoro-2-(((3-(phenylsulfonyl)phenyl)sulfonyl)methyl)-allyl)carbamate

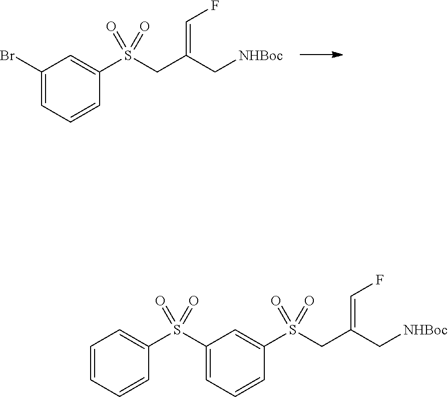 [SMILES: CC(C)(C)OC(=O)NC/C(=C\F)CS(=O)(=O)c1cccc(Br)c1 ; CC(C)(C)OC(=O)NC/C(=C\F)CS(=O)(=O)c1cccc(S(=O)(=O)c2ccccc2)c1]

A stirring mixture of tert-butyl (E)-(2-(((3-bromophenyl)sulfonyl)methyl)-3-fluoroallyl)carbamate (500 mg, 1.22 mmol), sodium carbonate (130 mg, 1.22 mmol), sodium benzenesulfinate (402 mg, 2.45 mmol), N,N-dimethylethylenediamine (3.0 mg, 34 μmol) and cupric acetate (3.0 mg, 17 μmol) in DMSO (2.0 mL) was purged with argon. The mixture was then heated to 100 °C for 4 h. LCMS analysis after this time indicated approximately 20% desired product. The reaction mixture was cooled to rt and then diluted with water (20 mL). The product was extracted with ethyl acetate (3×20 mL). The combined organic extract was washed with brine, dried over Na₂SO₄ and then concentrated in vacuo. The crude material was purified by NP chromatography (CombiFlash® Redisep column 12 g silica; eluent: solvent A=cyclohexane; solvent B=ethyl acetate; 20% B for 1 min followed by gradient 20%-40% B over 3.5 min. held at 40% B for 1.5 min. Finally, gradient of 40%-60% B over 4 min. Flow rate 30 mL/min.) to afford tert-butyl (E)-(3-fluoro-2-(((3-(phenylsulfonyl)phenyl)sulfonyl)-methyl)allyl)carbamate (80 mg, 14%). ¹H NMR (300 MHz, CDCl₃) δ 8.48-8.45 (m, 1H), 8.27 (ddd, J=7.9, 1.8, 1.1 Hz, 1H), 8.11 (ddd, J=7.8, 1.8, 1.1 Hz, 1H), 8.03-7.95 (m, 2H), 7.78 (td, J=7.9, 0.5 Hz, 1H), 7.69-7.53 (m, 3H), 6.40 (d, J=80.2 Hz, 1H), 4.98 (s, 1H), 3.98-3.91 (m, 2H), 3.81-3.76 (m, 2H), 1.46 (s, 9H).

TABLE 3
Summary of procedures used for the preparation of Compounds 3-18 with corresponding analytical data.

| Com- | | | | |
|---|---|---|---|---|
| pound | | Pro- | | |
| (free- | Sodium arylsulfinate | cedures | Procedural modification(s)/ | |
| base) | starting material | used | Chromatographic purification details | Analytical data (xHCl salt) |
| 3 | 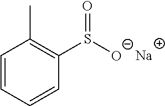 | A and B | Procedure A: crude material was purified by NP chromatography (CombiFlash ® Redisep column 12 g silica; eluent: solvent A = cyclohexane; solvent B = ethyl acetate; 5% B for 1 min followed by gradient 5%- 50% B over 10.5 min. Flow rate 30 mL/min.) | ¹H NMR (300 MHz, DMSO-d₆) δ 8.31 (s, 3H), 7.92-7.76 (m, 1H), 7.67 (td, J = 7.5, 1.4 Hz, 1H), 7.50 (d, J = 8.1 Hz, 2H), 6.88 (d, J = 81.4 Hz, 1H), 4.28 (d, J = 2.7 Hz, 2H), 3.57 (d, J = 2.5 Hz, 2H), 2.68 (s, 3H). LC-MS: m/z 244 [M + H]⁺; RT = 2.81 min (Method A). |
| 4 | 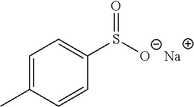 | A and B | None | ¹H NMR (300 MHz, DMSO-d₆) δ 8.41 (s, 3H), 7.87-7.70 (m, 2H), 7.59-7.38 (m, 2H), 6.81 (d, J = 81.6 Hz, 1H), 4.33 (d, J = 2.7 Hz, 2H), 3.57 (d, J = 2.7 Hz, 2H), 2.44 (s, 3H). LC-MS: m/z 244 [M + H]⁺; RT = 2.84 min (Method A). |
| 5 | 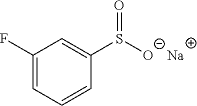 | E, A and B | Procedure E: Reaction is conducted over 4 h. Crude material is purified by RP chromatography (Reveleris ® C18 40 g; eluent: solvent A = 12 mM aq HCl; solvent B = methanol; gradient 0-5% B over 2 min. followed by gradient 5-12% B over 1.5 min. Finally, held at 97% B for 3.5 min. | ¹H NMR (300 MHz, DMSO-d₆) δ 8.33 (s, 3H), 7.86-7.58 (m, 4H), 6.83 (d, J = 81.3 Hz, 1H), 4.42 (d, J = 2.7 Hz, 2H), 3.61 (d, J = 2.4 Hz, 2H). LC-MS: m/z 248 [M + H]⁺; RT = 2.59 min (Method A). |
| | | | Flow rate 40 mL/min.). | |
| | | | Procedure A: crude material was purified | |
| | | | by NP chromatography (CombiFlash ® | |
| | | | Redisep column 12 g silica; eluent: solvent | |
| | | | A = cyclohexane; solvent B = ethyl acetate; | |
| | | | 10% B for 1 min followed by gradient 10%- | |
| | | | 75% B over 11.5 min. Flow rate 30 | |
| | | | mL/min.) | |
| 6 | 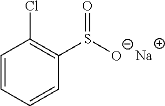 | A and B | Procedure A: crude material was purified by NP chromatography (CombiFlash ® Redisep column 12 g silica; eluent: solvent A = cyclohexane; solvent B = ethyl acetate; 5% B for 1 min followed by gradient 5%- 40% B over 9 min. Flow rate 30 mL/min.) | ¹H NMR (300 MHz, DMSO-d₆) δ 8.11 (brs, 3H), 8.02 (dd, J = 8.4, 1.0 Hz, 1H), 7.81 (dd, J = 3.9, 1.6 Hz, 2H), 7.67 (ddd, J = 7.9, 5.6, 3.1 Hz, 1H), 6.96 (d, J = 80.7 Hz, 1H), 4.42 (s, 2H), 3.62 (s, 2H). LC-MS: m/z 264 [M + H]⁺; RT = 2.63 min (Method A). |
| 7 | 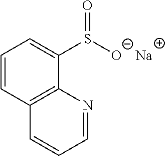 [SMILES: O=S([O-])c1cccc2cccnc12 ; [Na+]] | A and B | Procedure A: the product did not precipitate upon addition of water. The product was instead extracted into ethyl acetate. Product was obtained after removal of volatiles in vacuo. | ¹H NMR (300 MHz, DMSO-d₆) δ 9.14 (dd, J = 4.3, 1.7 Hz, 1H), 8.62 (dd, J = 8.4, 1.8 Hz, 1H), 8.42 (ddd, J = 8.7, 7.8, 1.4 Hz, 2H), 7.91-7.82 (m, 1H), 7.78 (dd, J = 8.4, 4.2 Hz, 1H), 6.73 (d, J = 80.8 Hz, 1H), 4.78 (d, J = 2.7 Hz, 2H), 3.65 (d, J = 2.4 Hz, 2H). LC-MS: m/z 281 [M + H]⁺; RT = 2.67 min (Method A). |
| 8 | 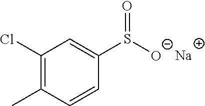 | E, A and B | Procedure E: no chromatographic purification performed. Crude product used. Procedure A: the product did not precipitate upon addition of water. The product was instead extracted into ethyl acetate. Product was obtained after removal of volatiles in vacuo. Crude material was subjected to | ¹H NMR (300 MHz, DMSO-d₆) δ 8.35 (s, 3H), 7.93 (d, J = 1.9 Hz, 1H), 7.78 (dd, J = 8.0, 1.9 Hz, 1H), 7.69 (d, J = 8.0 Hz, 1H), 6.85 (d, J = 81.4 Hz, 1H), 4.40 (d, J = 2.7 Hz, 2H), 3.61 (d, J = 2.4 Hz, 2H), 2.46 (s, 3H). LC-MS: m/z 278 [M + H]⁺; RT = 2.49 min (Method A). |
| | | | chromatographic purification | |
| | | | (CombiFlash ® Redisep column 12 g silica; | |
| | | | eluent: solvent A = cyclohexane; solvent B = | |
| | | | ethyl acetate; 10% B for 1 min followed | |
| | | | by gradient 10%-70% B over 8 min. Flow | |
| | | | rate 30 mL/min.) | |
| 9 | 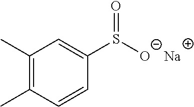 | | Procedure E: Reaction was conducted overnight during which time the product precipitated from solution. Crude material was purified by RP chromatography (Reveleris ® C18 40 g; eluent: solvent A = 12 mM aq HCl; solvent B = methanol; 20% B for 5 min. followed by gradient 20- | ¹H NMR (300 MHz, DMSO-d₆) δ 8.39 (s, 4H), 7.69 (s, 1H), 7.61 (dd, J = 7.9, 2.0 Hz, 1H), 7.44 (d, J = 8.0 Hz, 1H), 6.83 (d, J = 81.6 Hz, 1H), 4.30 (d, J = 2.7 Hz, 2H), 3.57 (s, 3H), 2.34 (s, 6H). LC-MS: m/z 258 [M + H]⁺; RT = 3.00 min (Method A). |
| | | | 90% B over 1 min. followed by gradient 5- | |
| | | | 12% B over 1.5 min. Finally, held at 90% B | |
| | | | for 10 min. Flow rate 40 mL/min.). | |
| | | | Procedure A: the product did not precipitate | |
| | | | upon addition of water. The product was | |
| | | | instead extracted into ethyl acetate. Product | |
| | | | was obtained after removal of volatiles in | |
| | | | vacuo. Crude material was subjected to NP | |
| | | | chromatographic purification | |
| | | | (CombiFlash ® Redisep column 12 g silica; | |
| | | | eluent: solvent A = cyclohexane; solvent B = | |
| | | | ethyl acetate; 10% B for 1 min followed | |
| | | | by gradient 10%-45% B over 8 min. Flow | |
| | | | rate 30 mL/min.) | |
| 10 | 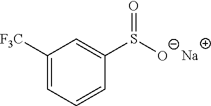 [SMILES: O=S([O-])c1cccc(C(F)(F)F)c1 ; [Na+]] | E, A and B | Procedure E: Reaction is conducted overnight during which time the product precipitated from solution. This suspension was used directly in the next step. Procedure A: A suspension of the sodium sulfinate starting material was used. The product did not precipitate upon addition of | ¹H NMR (300 MHz, DMSO-d₆) δ 8.33 (s, 3H), 8.28-8.12 (m, 3H), 7.97 (t, J = 8.1 Hz, 1H), 6.84 (d, J = 81.2 Hz, 1H), 4.48 (d, J = 2.7 Hz, 2H), 3.65 (d, J = 2.4 Hz, 2H). LC-MS: m/z 298 [M + H]⁺; RT = 3.00 min (Method A). |
| | | | water. The product was instead, extracted | |
| | | | into ethyl acetate. Product was obtained | |
| | | | after removal of volatiles in vacuo. Crude | |
| | | | material was purified by recrystallization | |
| | | | from hot cyclohexane | |
| 11 | 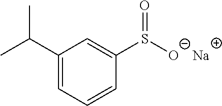 | E, A and B | Procedure E: the reaction mixture was concentrated to dryness and the the solid thus obtained was used in the next step without purification Procedure A: crude material was purified by NP chromatography (CombiFlash ® Redisep column 12 g silica; eluent: solvent | ¹H NMR (300 MHz, DMSO-d₆) δ 8.21 (s, 3H), 7.74 (s, 1H), 7.71 (dt, J = 2.7, 1.6 Hz, 1H), 7.68 (d, J = 1.6 Hz, 1H), 7.66-7.58 (m, 1H), 6.79 (d, J = 81.6 Hz, 1H), 4.30 (s, 2H), 3.66-3.53 (m, 2H), 3.06 (h, J = 6.9 Hz, 1H), 1.25 (d, J = 6.9 Hz, 6H). LC-MS: m/z 272 |
| | | | A = cyclohexane; solvent B = ethyl acetate; | [M + H]⁺; RT = 3.28 min (Method A). |
| | | | 0% B for 1 min followed by gradient 0%- | |
| | | | 25% B over 7.5 min. then held at 25% B for | |
| | | | 1.5 min. Finally, gradient 25%-50% over 6 | |
| | | | min. Flow rate 30 mL/min.) | |
| | | | Procedure B: Ethyl acetate used as the | |
| | | | reaction solvent instead of MeOH | |
| 12 | 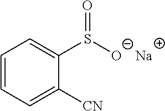 [SMILES: N#Cc1ccccc1S(=O)[O-] ; [Na+]] | E, A and B | Procedure E: the reaction mixture was concentrated to dryness. MeOH was added and the precipitated inorganic impurities were removed by filtration. The filtrate was concentrated in vacuo to afford the desired product. The material thus obtained was used without further purification | ¹H NMR (300 MHz, DMSO-d₆) δ 8.42 (s, 3H), 8.27-8.17 (m, 1H), 8.17-8.08 (m, 1H), 8.08-7.95 (m, 2H), 6.79 (d, J = 80.9 Hz, 1H), 4.52 (d, J = 2.6 Hz, 2H), 3.66 (s, 2H). LC-MS: m/z 255 [M + H]⁺; RT = 2.49 min (Method A). |
| | | | Procedure A: crude material was purified | |
| | | | by NP chromatography CombiFlash ® | |
| | | | Redisep column 12 g silica; eluent: solvent | |
| | | | A = cyclohexane; solvent B = ethyl acetate; | |
| | | | 20% B for 1 min followed by gradient 20%- | |
| | | | 60% B over 5 min. Finally, held at 60% B | |
| | | | for 2 min. Flow rate 30 mL/min.) | |
| 13 | 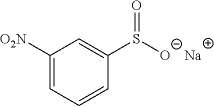 [SMILES: O=[N+]([O-])c1cccc(S(=O)[O-])c1 ; [Na+]] | E, A and B | Procedure E: the reaction mixture was concentrated to dryness. MeOH was added and the precipitated inorganic impurities were removed by filtration. The filtrate was concentrated in vacuo to afford the desired product. The material thus obtained was used without further purification | ¹H NMR (300 MHz, DMSO-d₆) δ 8.63 (dd, J = 4.2, 2.3 Hz, 2H), 8.51-8.11 (m, 4H), 8.00 (dd, J = 8.9, 7.8 Hz, 1H), 6.85 (d, J = 81.0 Hz, 1H), 4.51 (d, J = 2.6 Hz, 2H), 3.64 (s, 2H). LC-MS: m/z 275 [M + H]⁺; RT = 2.65 min (Method A). |
| | | | Procedure A: the product did not precipitate | |
| | | | upon addition of water. The product was | |
| | | | instead extracted into ethyl acetate. Product | |
| | | | was obtained after removal of volatiles in | |
| | | | vacuo. | |
| 14 | 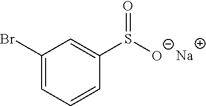 | E, A, L and B | Procedure E: the reaction mixture was concentrated to dryness. MeOH was added and the precipitated inorganic impurities were removed by filtration. The filtrate was concentrated in vacuo to afford the desired product. The material thus obtained was used without further purification | ¹H NMR (300 MHz, DMSO-d₆) δ 8.44- 8.29 (m, 5H), 8.25 (dt, J = 7.9, 1.4 Hz, 1H), 8.10-8.01 (m, 2H), 7.96 (t, J = 7.8 Hz, 1H), 7.80-7.71 (m, 1H), 7.72- 7.62 (m, 2H), 6.78 (d, J = 81.2 Hz, 1H), 4.57-4.37 (m, 2H), 3.62 (s, 2H). LC- MS: m/z 370 [M + H]⁺; RT = 3.14 min |
| | | | Procedure A: the product did not precipitate | (Method A). |
| | | | upon addition of water. The product was | |
| | | | instead extracted into ethyl acetate. Product | |
| | | | was obtained after removal of volatiles in | |
| | | | vacuo. Crude material was subjected to NP | |
| | | | chromatographic purification | |
| | | | (CombiFlash ® Redisep column 40 g silica; | |
| | | | eluent: solvent A = cyclohexane; solvent | |
| | | | B = ethyl acetate; 0% B for 1 min followed | |
| | | | by gradient 0%-25% B over 7.5 min. then | |
| | | | held at 25% B for 4 min. Finally, held at | |
| | | | 80% B for 6 min. Flow rate 40 mL/min.) | |
| 15 | 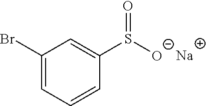 | E, A and B | Procedure E: the reaction mixture was concentrated to dryness. MeOH was added and the precipitated inorganic impurities were removed by filtration. The filtrate was concentrated in vacuo to afford the desired product. The material thus obtained was used without further purification | ¹H NMR (300 MHz, DMSO-d₆) δ 8.40 (s, 3H), 8.09 (t, J = 1.9 Hz, 1H), 8.02 (ddd, J = 8.0, 2.0, 1.0 Hz, 1H), 7.93 (dt, J = 7.9, 1.3 Hz, 1H), 7.66 (t, J = 7.9 Hz, 1H), 6.86 (d, J = 81.3 Hz, 1H), 4.45 (d, J = 2.6 Hz, 2H), 3.76-3.48 (m, 2H). LC-MS: m/z 308 [M + H]⁺; RT = 3.00 |
| | | | Procedure A: the product did not precipitate | min (Method A). |
| | | | upon addition of water. The product was | |
| | | | instead extracted into ethyl acetate. Product | |
| | | | was obtained after removal of volatiles in | |
| | | | vacuo. Crude material was subjected to NP | |
| | | | chromatographic purification | |
| | | | (CombiFlash ® Redisep column 12 g silica; | |
| | | | eluent: solvent A = cyclohexane; solvent | |
| | | | B = ethyl acetate; 5% B for 1 min followed | |
| | | | by gradient 5%-60% B over 7 min. | |
| | | | Flow rate 30 mL/min.) | |
| 16 | 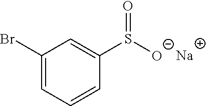 | E, A, H, I and B | Procedure E: the reaction mixture was concentrated to dryness. MeOH was added and the precipitated inorganic impurities were removed by filtration. The filtrate was concentrated in vacuo to afford the desired product. The material thus obtained was used without further purification | ¹H NMR (300 MHz, DMSO-d₆) δ 8.46 (s, 3H), 8.43 (t, J = 1.8 Hz, 1H), 8.37- 8.30 (m, 1H), 8.04 (d, J = 3.2 Hz, 1H), 8.02-7.98 (m, 0H), 7.95 (d, J = 3.2 Hz, 1H), 7.83 (t, J = 7.8 Hz, 1H), 6.89 (d, J = 81.4 Hz, 1H), 4.48 (d, J = 2.6 Hz, 2H), 3.69-3.58 (m, 2H). LC-MS: m/z |
| | | | Procedure A: the product did not precipitate | 313 [M + H]⁺; RT = 3.07 min (Method |
| | | | upon addition of water. The product was | A). |
| | | | instead extracted into ethyl acetate. Product | |
| | | | was obtained after removal of volatiles in | |
| | | | vacuo. Crude material was subjected to NP | |
| | | | chromatographic purification | |
| | | | (CombiFlash ® Redisep column 12 g silica; | |
| | | | eluent: solvent A = cyclohexane; solvent | |
| | | | B = ethyl acetate; 5% B for 1 min followed | |
| | | | by gradient 5%-60% B over 7 min. | |
| | | | Flow rate 30 mL/min.) | |
| 17 | 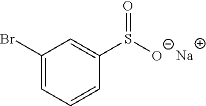 | E, A, H, J and B | Procedure E: the reaction mixture was concentrated to dryness. MeOH was added and the precipitated inorganic impurities were removed by filtration. The filtrate was concentrated in vacuo to afford the desired product. The material thus obtained was used without further purification | ¹H NMR (300 MHz, DMSO-d₆) δ 8.37 (s, 4H), 8.29 (s, 1H), 8.11 (d, J = 1.8 Hz, 1H), 8.03 (dt, J = 7.7, 1.4 Hz, 1H), 7.92- 7.81 (m, 1H), 7.75 (t, J = 7.8 Hz, 1H), 6.87 (d, J = 81.4 Hz, 1H), 4.54-4.37 (m, 2H), 3.63 (s, 2H), 2.72 (s, 3H). LC- MS: m/z 327 [M + H]⁺; RT = 3.23 min |
| | | | Procedure A: the product did not precipitate | (Method A). |
| | | | upon addition of water. The product was | |
| | | | instead extracted into ethyl acetate. Product | |
| | | | was obtained after removal of volatiles in | |
| | | | vacuo. Crude material was subjected to NP | |
| | | | chromatographic purification | |
| | | | (CombiFlash ® Redisep column 12 g silica; | |
| | | | eluent: solvent A = cyclohexane; solvent | |
| | | | B = ethyl acetate; 5% B for 1 min followed | |
| | | | by gradient 5%-60% B over 7 min. | |
| | | | Flow rate 30 mL/min.) | |
| 18 | 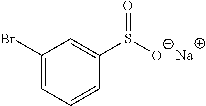 | E, A, H, K and B | Procedure E: the reaction mixture was concentrated to dryness. MeOH was added and the precipitated inorganic impurities were removed by filtration. The filtrate was concentrated in vacuo to afford the desired product. The material thus obtained was used without further purification | ¹H NMR (300 MHz, DMSO-d₆) δ 7.52 (t, J = 2.0 Hz, 1H), 7.48 (d, J = 2.5 Hz, 1H), 7.32 (ddd, J = 7.9, 2.4, 1.3 Hz, 1H), 7.08-6.91 (m, 2H), 6.05 (d, J = 80.4 Hz, 1H), 5.62 (d, J = 2.5 Hz, 1H), 3.37 (d, J = 2.8 Hz, 2H), 3.08 (d, J = 2.5 Hz, 2H), 1.58 (s, 3H). LC-MS: m/z 310 |
| | | | Procedure A: the product did not precipitate | [M + H]⁺; RT = 3.13 min (Method A). |
| | | | upon addition of water. The product was | |
| | | | instead, extracted into ethyl acetate. Product | |
| | | | was obtained after removal of volatiles in | |
| | | | vacuo. Crude material was subjected to NP | |
| | | | chromatographic purification | |
| | | | (CombiFlash ® Redisep column 12 g silica; | |
| | | | eluent: solvent A = cyclohexane; solvent | |
| | | | B = ethyl acetate; 5% B for 1 min followed | |
| | | | by gradient 5%-60% B over 7 min. Flow | |
| | | | rate 30 mL/min.) | |

### Preparation of (E)-2-(((3-chlorophenyl)sulfonyl)methyl)-3-fluoroprop-2-en-1-amine hydrochloride (Compound 19)

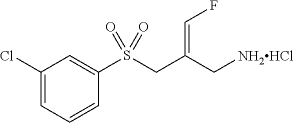

### Procedure M: Preparation of tert-butyl (E)-(2-(((3-chlorophenyl)thio)methyl)-3-fluoroallyl)carbamate

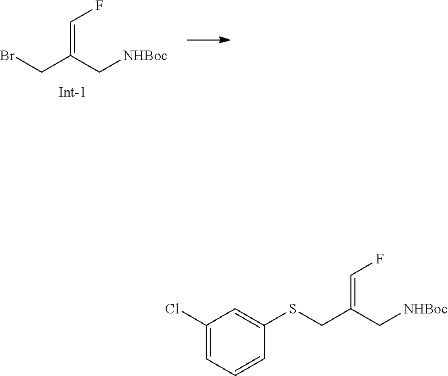 [SMILES: CC(C)(C)OC(=O)NC/C(=C\F)CBr ; CC(C)(C)OC(=O)NC/C(=C\F)CSc1cccc(Cl)c1]

To a stirred mixture of cesium carbonate (456 mg, 1.40 mmol) and 3-chlorothiophenol (217 mg, 1.50 mmol) in DMF (2.5 mL) was added Int-1 (268 mg, 1.00 mmol). The resulting mixture was stirred at rt for 2 h. TLC analysis after this time showed complete consumption of Int-1. Water (10 mL) was added and the product was extracted with ethyl acetate (3×10 mL). The combined organic extract was washed with brine, dried over Na₂SO₄ and concentrated to afford tert-butyl (E)-(2-(((3-chlorophenyl)thio)methyl)-3-fluoroallyl)carbamate (350 mg). This material was used directly in the next step.

### Procedure N: Preparation of tert-butyl (E)-(2-(((3-chlorophenyl)sulfonyl)methyl)-3-fluoroallyl)-carbamate

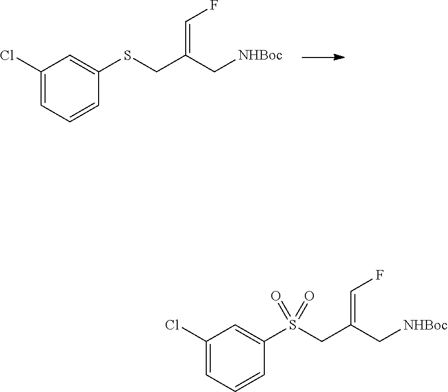 [SMILES: CC(C)(C)OC(=O)NC/C(=C\F)CSc1cccc(Cl)c1 ; CC(C)(C)OC(=O)NC/C(=C\F)CS(=O)(=O)c1cccc(Cl)c1]

To a stirring solution of tert-butyl (E)-(2-(((3-chlorophenyl)thio)methyl)-3-fluoroallyl)carbamate (350 mg) in methanol (5 mL) was added hydrogen peroxide (30% w/v, 4.0 mL) followed by sodium tungstate (100 mg). The mixture was stirred at rt for 2 h. LCMS analysis after this time indicated complete consumption of starting material. Water (20 mL) was added and the product was extracted with ethyl acetate (3×10 mL). The combined organic extract was washed with brine, dried over Na₂SO₄ and then concentrated in vacuo. The residue was purified over silica gel (CombiFlash® Redisep column 12 g silica; eluent: solvent A=cyclohexane; solvent B=ethyl acetate; 100% cyclohexane for 1 min. followed by gradient 0%-40% ethyl acetate in cyclohexane over 7 min. Flow rate 30 m/min.) to afford tert-butyl (E)-(2-(((3-chlorophenyl)sulfonyl)methyl)-3-fluoroallyl)-carbamate (0.31 g, 91%). ¹H NMR (300 MHz, CDCl₃) δ 7.92 (t, J=1.9 Hz, 1H), 7.81 (dt, J=7.8, 1.4 Hz, 1H), 7.69 (ddd, J=8.1, 2.1, 1.1 Hz, 1H), 7.56 (t, J=7.9 Hz, 1H), 6.46 (d, J=80.4 Hz, 1H), 5.02 (s, 1H), 4.00 (ddd, J=6.4, 2.8, 1.3 Hz, 2H), 3.81-3.71 (m, 2H), 1.47 (s, 9H).

### Procedure O: Preparation of (E)-2-(((3-chlorophenyl)sulfonyl)methyl)-3-fluoroprop-2-en-1-amine hydrochloride (Compound 19)

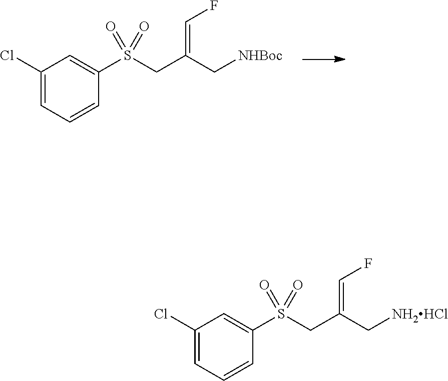

To a suspension of tert-butyl (E)-(2-(((3-chlorophenyl)sulfonyl)methyl)-3-fluoroallyl)-carbamate (0.33 g, 0.90 mmol) in methanol (5.0 mL) was added HCl (2.0 M in diethyl ether; 5.0 mL, 10.0 mmol). The resulting mixture was stirred at rt for 2 h. TLC analysis after this time showed completion of the reaction. The reaction mixture was concentrated under vacuum. Ethyl acetate (5 mL) was added and the mixture was stirred for 5 min. The precipitated product was collected by filtration and the filter cake was washed with ethyl acetate (5 mL). The solid was dried at 60 °C for 1 h to give (E)-2-(((3-chlorophenyl)sulfonyl)methyl)-3-fluoroprop-2-en-1-amine hydrochloride (190 mg, 70%). ¹H NMR (300 MHz, DMSO-d₆) δ 8.35 (s, 3H), 7.98 (t, J=1.9 Hz, 1H), 7.90 (ddt, J=8.3, 4.2, 1.3 Hz, 2H), 7.73 (t, J=7.9 Hz, 1H), 6.85 (d, J=81.3 Hz, 1H), 4.44 (d, J=2.6 Hz, 2H), 3.62 (d, J=2.4 Hz, 2H). LC-MS: m/z 264 [M+H]+; RT=2.83 min (Method A).

### Procedure P: Preparation of 2-(3-nitrophenyl)-2H-1,2,3-triazole

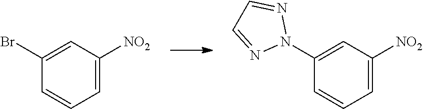 [SMILES: O=[N+]([O-])c1cccc(Br)c1 ; O=[N+]([O-])c1cccc(-n2nccn2)c1]

A stirring mixture of 1-bromo-3-nitrobenzene (1.01 g, 5.00 mmol), potassium phosphate tribasic (2.12 g, 10.0 mmol), 2H-triazole (691 mg, 10.0 mmol), N,N′-dimethylethylenediamine (44 mg, 0.50 mmol), cuprous iodide (17 μL, 0.50 mmol), cuprous oxide (72 mg, 0.50 mmol) and N,N′-dimethylethylenediamine (55 μL, 0.50 mmol) in DMF (25 mL) was purged with argon. The mixture was heated at reflux for 6 h. TLC analysis after this time showed formation of the desired product and a trace of unreacted starting material. The reaction mixture was cooled to rt and diluted with water (70 mL). The mixture was stirred at rt for 5 min and the resulting solid was collected by filtration. The crude solid was purified over silica gel (CombiFlash® Redisep column 24 g silica; eluent: solvent A=cyclohexane; solvent B=ethyl acetate; 10% B for 1 min followed by gradient 10%-16% B over 1 min. then held at 16% B for 3 min. then gradient 16%-50% B for 1 min. Finally, gradient of 50%-60% B over 9 min. Flow rate 30 mL/min.) to afford 2-(3-nitrophenyl)triazole (350 mg, 37%). ¹H NMR (300 MHz, CDCl₃) δ 9.00 (t, J=2.2 Hz, 1H), 8.47 (ddd, J=8.2, 2.1, 1.0 Hz, 1H), 8.24 (ddd, J=8.2, 2.2, 1.0 Hz, 1H), 7.91 (s, 2H), 7.71 (t, J=8.2 Hz, 1H).

### Procedure Q: Preparation of 3-(2H-1,2,3-triazol-2-yl)aniline

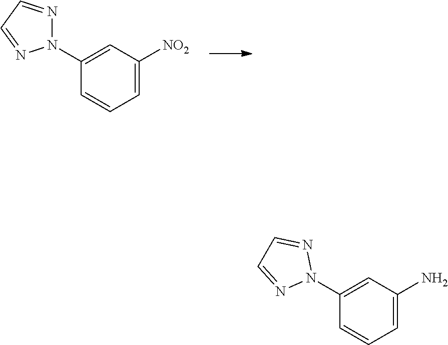 [SMILES: O=[N+]([O-])c1cccc(-n2nccn2)c1 ; Nc1cccc(-n2nccn2)c1]

A mixture of 2-(3-nitrophenyl)triazole (350 mg, 1.84 mmol) and 10% (w/w) Pd/C (40 mg) in methanol (25 mL) was hydrogenated at 50 psi for 1 h. TLC analysis showed absence of the starting material and formation of the lower running product. Pd/C was removed by filtration and the filtrate was concentrated under vacuum to afford crude 3-(triazol-2-yl)aniline (300 mg, 1.87 mmol, quantitative yield). This material was used in the next step without purification.

### Procedure R: Preparation of 3-(2H-1,2,3-triazol-2-yl)benzenethiol

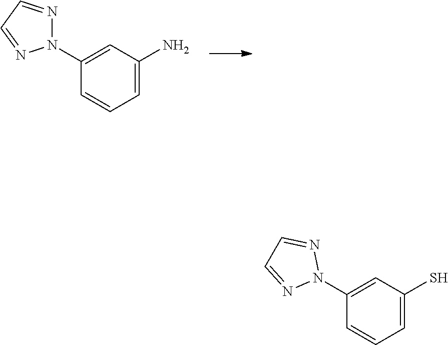 [SMILES: Nc1cccc(-n2nccn2)c1 ; Sc1cccc(-n2nccn2)c1]

A stirring solution of crude 3-(2H-1,2,3-triazol-2-yl)aniline (300 mg, 1.87 mmol) and conc. aqueous HCl (0.47 mL, 5.62 mmol), water (5.0 mL) and THF (5.0 mL) was cooled to 0 °C. A solution of sodium nitrite (142 mg, 2.06 mmol) in water (2.0 mL) was added in 4 lots. The resulting mixture was stirred at 0 °C for 30 min. A solution of potassium ethyl xanthogenate (453 mg, 2.81 mmol) in water (2.0 mL) was then added and the mixture was stirred at 0 °C for 20 min. The mixture was gradually warmed to 60 °C over 15 min. The mixture was then cooled to rt before aq. NaOH (2.0 M; 3.0 mL) was added. Stirring was then continued at rt for 1 h. The reaction mixture was concentrated under vacuum to approximately 2 mL. This solution was used directly in the next step without isolation or purification.

### Procedure S: Preparation of 1-(3-(methylsulfonyl)phenoxy)-2-nitrobenzene

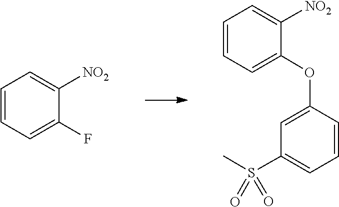 [SMILES: O=[N+]([O-])c1ccccc1F ; CS(=O)(=O)c1cccc(Oc2ccccc2[N+](=O)[O-])c1]

A mixture of 3-(methylsulfonyl)phenol (344 mg, 2.00 mmol) and potassium carbonate (300 mg, 2.17 mmol) in DMF (3.0 mL) was stirred for 5 min. To this was added 1-fluoro-2-nitro-benzene (282 mg, 2.00 mmol) in one lot and the resulting mixture was heated at 100 °C for 2 hours. TLC analysis after this time showed completion of the reaction. The reaction mixture was cooled to rt and then diluted with water (30 mL). The product was extracted with ethyl acetate (3×25 mL). The combined organic extract was washed with brine, dried over Na₂SO₄ and then concentrated in vacuo to afford 1-(3-(methylsulfonyl)phenoxy)-2-nitrobenzene (530 mg, 90%). ¹H NMR (300 MHz, CDCl₃) δ 8.04 (dd, J=8.1, 1.7 Hz, 1H), 7.75 (ddd, J=7.8, 1.7, 1.0 Hz, 1H), 7.69-7.52 (m, 3H), 7.42-7.30 (m, 2H), 7.16 (dd, J=8.3, 1.2 Hz, 1H).

### Procedure T: Preparation of 2-(3-(methylsulfonyl)phenoxy)aniline

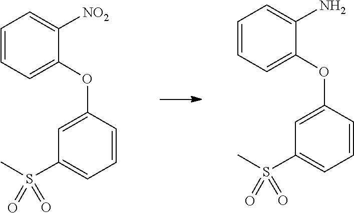 [SMILES: CS(=O)(=O)c1cccc(Oc2ccccc2[N+](=O)[O-])c1 ; CS(=O)(=O)c1cccc(Oc2ccccc2N)c1]

To a stirring solution of 1-(3-(methylsulfonyl)phenoxy)-2-nitrobenzene (530 mg, 1.81 mmol), THF (20 mL) and sat. aq. NH₄Cl (10 mL) was added zinc powder (1.00 g). The resulting suspension was stirred at rt for 30 min. LCMS analysis after this time indicated complete consumption of starting material. Saturated aq. NaHCO₃ (10 mL) and ethyl acetate (20 mL) were added and the mixture was stirred for 5 min. The organic layer was separated, washed with brine, dried over Na₂SO₄ and then concentrated in vacuo to afford 2-(3-(methylsulfonyl)phenoxy)aniline (450 mg, 95%). This material was used directly in the next step without further purification.

### Preparation of (E)-3-((2-(aminomethyl)-3-fluoroallyl)sulfonyl)benzamide hydrochloride (Compound 27)

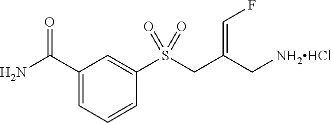

### Procedure U: Preparation of (E)-3-((2-(aminomethyl)-3-fluoroallyl)sulfonyl)benzamide hydrochloride (Compound 27)

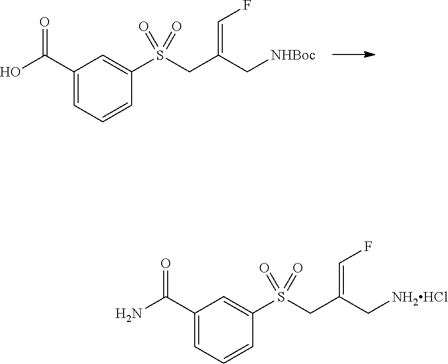

A mixture of (E)-3-((2-(((tert-butoxycarbonyl)amino)methyl)-3-fluoroallyl)sulfonyl)benzoic acid (150 mg, 0.40 mmol), ammonium chloride (43.0 mg, 0.80 mmol) and N,N-diisopropylethylamine (0.28 mL, 1.61 mmol) in DMF (2.0 mL) was stirred for 10 min. HATU (229 mg, 0.60 mmol) was added and stirring was continued at rt for 1 h. LCMS analysis after this time showed completion of the reaction. The reaction mixture was directly injected onto a 40 g RP column and purified by chromatography (Reveleris® C18 40 g; eluent: solvent A=12 mM aq HCl; solvent B=methanol; 5% B for 3 min followed by gradient 5-65% B over 15 min. Flow rate 40 mL/min.). Fractions containing the desired product were combined and concentrated under vacuum. During concentration, deprotection of the —NHBoc occurred to afford the hydrochloride salt. After complete removal of the water under vacuum, acetonitrile (4 mL) was added and the mixture was stirred for 5 min. The mixture, now containing precipitated product, was transferred to a vial and the solid was spun down in a centrifuge (4000 rpm, 4 min). The liquid was carefully decanted and the solid residue was dried at 60 °C for 1 h to afford Compound 27 (65 mg, 52%). ¹H NMR (300 MHz, DMSO-d₆) δ 8.44 (d, J=1.7 Hz, 1H), 8.35 (d, J=12.5 Hz, 4H), 8.29-8.20 (m, 1H), 8.10-7.96 (m, 1H), 7.79 (t, J=7.8 Hz, 1H), 6.85 (d, J=81.4 Hz, 1H), 4.41 (d, J=2.6 Hz, 2H), 3.61 (d, J=4.9 Hz, 2H). LC-MS: m/z 273 [M+H]P; RT=2.82 min (Method A).

### Preparation of (E)-4-((2-(aminomethyl)-3-fluoroallyl)sulfonyl)benzoic acid hydrochloride (Compound 31)

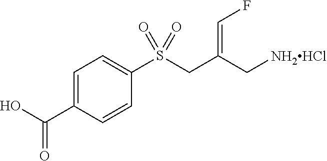

### Procedure V: Preparation of (E)-4-((2-(((tert-butoxycarbonyl)amino)methyl)-3-fluoroallyl)sulfonyl)benzoic acid

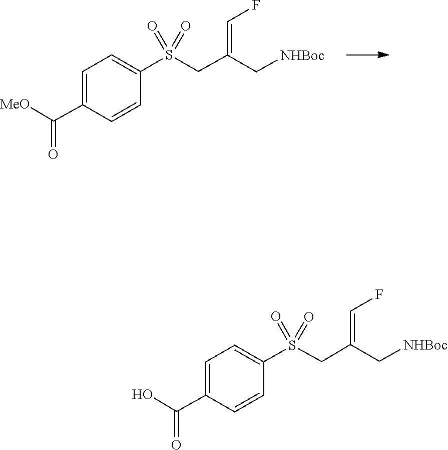 [SMILES: COC(=O)c1ccc(S(=O)(=O)C/C(=C/F)CNC(=O)OC(C)(C)C)cc1 ; CC(C)(C)OC(=O)NC/C(=C\F)CS(=O)(=O)c1ccc(C(=O)O)cc1]

To a stirred solution of methyl (E)-4-((2-(((tert-butoxycarbonyl)amino)methyl)-3-fluoroallyl)sulfonyl)benzoate (340 mg, 0.88 mmol) in methanol (10 mL) was added aq. NaOH (2.0 M; 2.50 mL, 5.00 mmol). The resulting mixture was stirred at rt for 1 h. LCMS analysis after this time showed complete hydrolysis of the methyl ester. The reaction mixture was then concentrated under vacuum. Water (10 mL) was added to dissolve the residue. The mixture was acidified with 2 M HCl when a white solid precipitated. The solid product was collected by filtration and the solid was washed with water and then dried at 60 °C to afford (E)-4-((2-(((tert-butoxycarbonyl)amino)methyl)-3-fluoroallyl)sulfonyl)benzoic acid (328 mg, 100%). This material was used directly in the next step without further purification.

### Procedure W: Preparation of (E)-4-((2-(aminomethyl)-3-fluoroallyl)sulfonyl)benzoic acid hydrochloride (Compound 31)

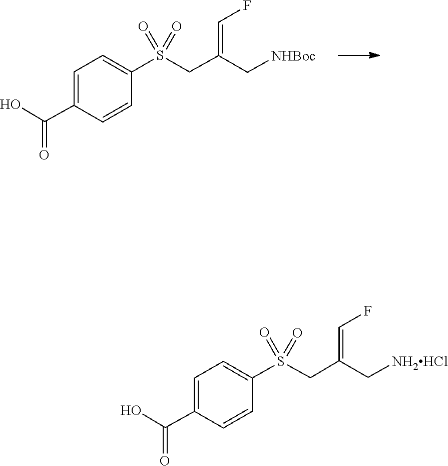

To a stirring suspension of (E)-4-((2-(((tert-butoxycarbonyl)amino)methyl)-3-fluoroallyl)sulfonyl)benzoic acid (328 mg, 0.88 mmol) in dichloromethane (5 mL)was added TFA (5.0 mL). The resulting mixture was stirred at rt for 1 h. LCMS analysis after this time indicated complete consumption of starting material. The reaction mixture was then concentrated under vacuum. Ethyl acetate (10 mL) was added and the mixture was again concentrated under vacuum. This process was repeated twice more in order to ensure complete removal of residual TFA. The residue was then dissolved in water (2 mL). To this was added aq. HCl (2.0 M, 2.0 mL) at which time a white precipitate formed. The resulting suspension was stirred for 5 min. The solid was collected by filtration and washed with water (1 mL). The product was dried at 60 °C for 2 h to afford Compound 31 (0.22 g, 81%). ¹H NMR (300 MHz, DMSO-d₆) δ 8.34 (s, 3H), 8.20 (d, J=8.4 Hz, 2H), 8.04 (d, J=8.4 Hz, 2H), 6.82 (d, J=81.2 Hz, 1H), 4.42 (d, J=2.6 Hz, 2H), 3.60 (d, J=2.3 Hz, 2H). LC-MS: m/z 274 [M+H]+(Method A).

TABLE 4
Summary of procedures used for the preparation of Compounds 20-35 with corresponding analytical data.

| Com- | | | | |
|---|---|---|---|---|
| pound | | Pro- | Procedural modification(s)/ | |
| (free- | Thiol starting | cedures | Chromatgraphic | |
| base) | material | used | purification details | Analytical data (xHCl salt) |
| 20 | 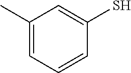 | M, N and O | Procedure N: Purified by NP chromatography (CombiFlash ® Redisep column 12 g silica; eluent: solvent A = cyclohexane; solvent B = ethyl acetate; 0% B for 1 min followed by gradient 0%-50% | ¹H NMR (300 MHz, DMSO-d₆) δ 8.48 (s, 3H), 7.79-7.73 (m, 1H), 7.73-7.67 (m, 1H), 7.64- 7.52 (m, 2H), 6.83 (d, J = 81.6 Hz, 1H), 4.37 (d, J = 2.7 Hz, 2H), 3.57 (s, 2H), 2.43 (s, 3H). LC-MS: m/z 244 [M + H]⁺; RT = 2.86 min |
| | | | B over 9 min. Flow rate 30 mL/min.) | (Method A). |
| 21 | 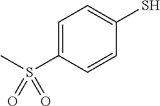 | M, N and O | Procedure M: crude material was purified by NP chromatography (CombiFlash ® Redisep column 12 g silica; eluent: solvent A = cyclohexane; solvent B = ethyl acetate; 10% B for 1 min followed by gradient 10%-60% B over 9 min. Finally, held at 60% for 3 min. Flow rate 30 mL/min.) | ¹H NMR (300 MHz, DMSO-d₆) δ 8.36 (s, 3H), 8.25 (d, J = 8.8 Hz, 2H), 8.19 (d, J = 8.8 Hz, 2H), 6.87 (d, J = 81.1 Hz, 1H), 4.62-4.34 (m, 2H), 3.62 (d, J = 2.3 Hz, 2H). LC-MS: m/z 308 [M + H]⁺; RT = 2.33 min (Method A). |
| | | | Procedure N: acetic acid and acetonitrile | |
| | | | (1:1) was employed as solvent instead of | |
| | | | MeOH. Product precipitated upon diluting | |
| | | | with water. Isolated by filtration and dried | |
| 22 | 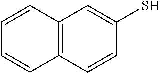 [SMILES: Sc1ccc2ccccc2c1] | M, N and O | Procedure M: Crude product precipitated upon diluting with water. Solid was isolated by filtration. The crude solid was purified by NP chromatography (CombiFlash ® Redisep column 12 g silica; eluent: solvent | ¹H NMR (300 MHz, DMSO-d₆) δ 8.60 (d, J = 1.8 Hz, 1H), 8.41 (s, 3H), 8.29-8.17 (m, 2H), 8.13 (dd, J = 8.1, 1.4 Hz, 1H), 7.93 (dd, J = 8.7, 1.9 Hz, 1H), 7.76 (dddd, J = 17.3, 8.2, 6.9, 1.4 Hz, 2H), 6.82 (d, J = 81.5 Hz, 1H), |
| | | | A = cyclohexane; solvent B = ethyl acetate; | 4.45 (d, J = 2.7 Hz, 2H), 3.62 (s, 2H). LC- |
| | | | 0% B for 4 min followed by gradient 0%- | MS: m/z 280 [M + H]⁺; RT = 3.18 min |
| | | | 30% B over 4 min. Finally, held at 30% for | (Method A). |
| | | | 3 min. Flow rate 30 mL/min.) | |
| | | | Procedure N: acetonitrile was employed as | |
| | | | solvent instead of MeOH. Product | |
| | | | precipitated upon diluting with water. | |
| | | | Product was isolated by filtration and dried | |
| 23 | 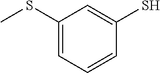 | M, N and O | Procedure N: product precipitated upon diluting with water. Product was isolated by filtration and dried | ¹H NMR (300 MHz, DMSO-d₆) δ 8.56-8.31 (m, 5H), 8.31-8.22 (m, 1H), 7.99 (t, J = 7.8 Hz, 1H), 6.86 (d, J = 81.2 Hz, 1H), 4.59-4.44 (m, 2H), 3.63 (s, 2H). LC-MS: m/z 308 [M + H]⁺; RT = 2.30 min (Method A). |
| 24 | 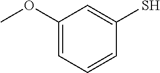 | | Procedure N: Purified by NP chromatography (CombiFlash ® Redisep column 12 g silica; eluent: solvent A = cyclohexane; solvent B = ethyl acetate; 10% B for 1 min followed by gradient | ¹H NMR (300 MHz, DMSO-d₆) δ 8.29 (s, 3H), 7.61 (t, J = 7.9 Hz, 1H), 7.47 (dt, J = 7.7, 1.2 Hz, 1H), 7.42 (d, J = 2.0 Hz, 1H), 7.36 (dt, J = 8.4, 1.7 Hz, 1H), 6.84 (d, J = 81.5 Hz, 1H), 4.35 (d, J = 3.9 Hz, 2H), 3.88 (s, 3H), 3.59 (d, |
| | | | 10%-60% B over 10 min. Flow rate 30 | J = 2.2 Hz, 2H). LC-MS: m/z 260 [M + H]⁺; |
| | | | mL/min.) | RT = 2.76 min (Method A). |
| 25 | 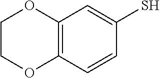 [SMILES: Sc1ccc2c(c1)OCCO2] | M, N and O | Procedure M: acetonitrile was employed as solvent instead of MeOH. Procedure N: Purified by NP chromatography (CombiFlash ® Redisep column 12 g silica; eluent: solvent A = | ¹H NMR (300 MHz, DMSO-d₆) δ 8.25 (s, 3H), 7.41-7.31 (m, 2H), 7.13 (d, J = 8.1 Hz, 1H), 6.84 (d, J = 81.6 Hz, 1H), 4.47-4.29 (m, 4H), 4.25 (d, J = 2.7 Hz, 2H), 3.59 (s, 2H). LC-MS: m/z 288 [M + H]⁺; 2.71 (Method A). |
| | | | cyclohexane; solvent B = ethyl acetate; | |
| | | | 10% B for 1 min followed by gradient | |
| | | | 10%-50% B over 5 min. Finally, held at | |
| | | | 50% for 2 min. Flow rate 30 mL/min.) | |
| 26 | 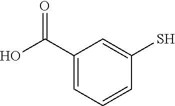 | M, N and O | Procedure M: acetone was employed as solvent instead of MeOH. The reaction mixture was concentrated under vacuum. Water and aq. 2M NaOH was added. The mixture was heated for 15 min at 50 °C. The reaction mixture was then cooled to rt and acidified to pH 5 with aq. 2M HCl. | ¹H NMR (300 MHz, DMSO-d₆) δ 13.65 (s, 1H), 8.39 (d, J = 1.8 Hz, 1H), 8.37-8.20 (m, 3H), 8.16 (dt, J = 7.9, 1.5 Hz, 1H), 7.84 (t, J = 7.8 Hz, 1H), 6.84 (d, J = 81.3 Hz, 1H), 4.40 (d, J = 2.6 Hz, 2H), 3.62 (s, 2H). LC-MS: m/z 274 [M + H]⁺;RT = 3.34 min (Method A). |
| | | | The product was extracted with ethyl | |
| | | | acetate. The combined organic layer was | |
| | | | dried over sodium sulfate and concentrated. | |
| | | | The residue was used in the next step | |
| | | | without purification. | |
| | | | Procedure N: product precipitated upon | |
| | | | diluting with water. Product was purified by | |
| | | | recrystallization from MeOH | |
| 28 | 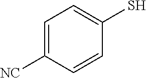 | M, N and O | Procedure N: Crude product precipitated upon diluting with water. Solid was isolated by filtration. The crude solid was purified by NP chromatography (CombiFlash ® Redisep column 12 g silica; eluent: solvent | ¹H NMR (300 MHz, DMSO-d₆) δ 8.38 (s, 3H), 8.26-8.15 (m, 2H), 8.15-8.05 (m, 2H), 6.83 (d, J = 81.1 Hz, 1H), 4.60-4.38 (m, 2H), 3.61 (s, 2H). LC-MS: m/z 255 [M + H]⁺; RT = 2.40 min (Method A). |
| | | | A = cyclohexane; solvent B = ethyl acetate; | |
| | | | 30% B for 1 min followed by gradient 30%- | |
| | | | 55% B over 6 min. Flow rate 30 mL/min.) | |
| 29 | 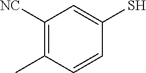 | | Procedure N: Crude product precipitated upon diluting with water. Solid was isolated by filtration. The crude solid was purified by NP chromatography (CombiFlash ® Redisep column 12 g silica; eluent: solvent | ¹H NMR (300 MHz, DMSO-d₆) δ 8.45-8.16 (m, 4H), 8.09 (dd, J = 8.2, 2.0 Hz, 1H), 7.79 (d, J = 8.2 Hz, 1H), 6.80 (d, J = 81.1 Hz, 1H), 4.41 (d, J = 3.0 Hz, 2H), 3.62 (d, J = 2.3 Hz, 2H), 2.62 (s, 3H). LC-MS: m/z 269 [M + H]⁺; RT = |
| | | | A = cyclohexane; solvent B = ethyl acetate; | 2.69 min (Method A). |
| | | | 10% B for 1 min followed by gradient 10%- | |
| | | | 60% B over 11 min. Finally, held at 60% | |
| | | | for 2 min. Flow rate 30 mL/min.) | |
| 30 | 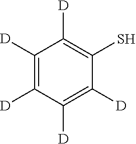 [SMILES: [2H]c1c([2H])c([2H])c(S)c([2H])c1[2H]] | M, N and O | Procedure M: crude material was purified by NP chromatography (CombiFlash ® Redisep column 40 g silica; eluent: solvent A = dichloromethane; solvent B = methanol; 0% B for 4 min followed by gradient 0%-20% B over 3 min. then held at 20% for 3.5 min. then gradient 20%- 100% B over 2 min. Finally, held at 100% for 3 min. Flow rate 40 mL/min.) | ¹H NMR (300 MHz, DMSO-d₆) δ 8.36 (s, 3H), 6.80 (d, J = 81.5 Hz, 1H), 4.46-4.23 (m, 2H), 3.59 (d, J = 2.4 Hz, 2H). LC-MS: m/z 235 [M + H]⁺; RT = 2.42 min (Method A). |
| | | | Procedure N: crude material was purified | |
| | | | by RP chromatography (Reveleris ® C18 40 | |
| | | | g; eluent: solvent A = 12 mM aq HCl; | |
| | | | solvent B = methanol; gradient 40-70% B | |
| | | | over 17 min. Finally, held at 100% B for 3 | |
| | | | min. Flow rate 40 mL/min.) | |
| 32 | 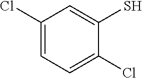 | M, N ad O | Procedure N: crude material was purified by NP chromatography (CombiFlash ® Redisep column 12 g silica; eluent: solvent A = cyclohexane; solvent B = ethyl acetate; 10% B for 1 min followed by gradient | ¹H NMR (300 MHz, DMSO-d₆) δ 8.18 (s, 3H), 7.98 (d, J = 2.5 Hz, 1H), 7.93 (dd, J = 8.5, 2.5 Hz, 1H), 7.84 (d, J = 8.5 Hz, 1H), 7.00 (d, J = 80.5 Hz, 1H), 4.48 (d, J = 2.6 Hz, 2H), 3.64 (dt, J = 9.5, 4.7 Hz, 2H). LC-MS: m/z 298 |
| | | | 10%-85% B over 12.5 min. Flow rate 30 | [M + H]⁺; RT = 3.06 min (Method A). |
| | | | mL/min.) | |
| 33 | 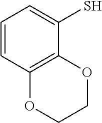 [SMILES: Sc1cccc2c1OCCO2] | | Procedure N: crude material was purified by NP chromatography (CombiFlash ® Redisep column 12 g silica; eluent: solvent A = cyclohexane; solvent B = ethyl acetate; 0% B for 1 min followed by gradient 0%- 40% B over 10 min. Flow rate 30 mL/min.) | ¹H NMR (300 MHz, DMSO-d₆) δ 8.28 (s, 3H), 7.32-7.28 (m, 1H), 7.28-7.24 (m, 1H), 7.05 (t, J = 8.0 Hz, 1H), 6.96 (d, J = 80.9 Hz, 1H), 4.53-4.42 (m, 2H), 4.42-4.35 (m, 2H), 4.33 (d, J = 2.6 Hz, 2H), 3.65-3.50 (m, 2H). LC- MS: m/z 288 [M + H]⁺; RT = 2.77 min (Method A). |
| 34 | 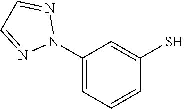 [SMILES: Sc1cccc(-n2nccn2)c1] | P, Q, R, M, N and O | Procedure M: crude material was purified by NP chromatography (CombiFlash ® Redisep column 24 g silica; eluent: solvent A = cyclohexane; solvent B = ethyl acetate; 0% B for 1 min followed by gradient 0%- 45% B over 15 min. Finally, held at 100% B for 2 min. Flow rate 35 mL/min.) | ¹H NMR (300 MHz, Methanol-d₄) δ 8.66 (t, J = 2.0 Hz, 1H), 8.52 (ddd, J = 8.1, 2.2, 1.1 Hz, 1H), 8.04 (s, 2H), 8.03-7.95 (m, 1H), 7.88 (t, J = 8.0 Hz, 1H), 6.89 (d, J = 80.3 Hz, 1H), 4.35-4.09 (m, 2H), 3.91 (d, J = 2.5 Hz, 2H). LC-MS: m/z 297 [M + H]⁺; RT = 3.02 min (Method A). |
| | | | Procedure N: Crude product precipitated | |
| | | | upon diluting with water. Solid was isolated | |
| | | | by filtration and dried at 60 °C for 2 h. No | |
| | | | further purification was performed | |
| 35 | 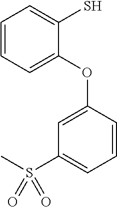 [SMILES: CS(=O)(=O)c1cccc(Oc2ccccc2S)c1] | S, T, R, M, N and O | Procedure R: The reaction mixture was acidified to pH 2 with 2M HCl prior to work-up. The product was extracted with ethyl acetate. The combined organic extract was washed with brine, dried over Na₂SO₄ and concentrated in vacuo. The crude material was purified by NP chromatography (CombiFlash ® Redisep column 12 g silica; eluent: solvent A = cyclohexane; solvent B = ethyl acetate; 10% B for 1 min followed by gradient 10%- 25% B over 3 min. then held at 25% for 2 min then gradient 25%-60% over 9 min. | ¹H NMR (300 MHz, Methanol-d₄) δ 8.08 (d, J = 7.9 Hz, 1H), 7.95-7.61 (m, 4H), 7.49 (t, J = 8.0 Hz, 2H), 7.21 (d, J = 8.3 Hz, 1H), 6.99 (d, J = 80.1 Hz, 1H), 4.35 (s, 2H), 3.86 (s, 2H), 3.18 (s, 3H). LC-MS: m/z 400 [M + H]⁺; RT = 2.88 min (Method A). |
| | | | Finally, held at 100% over 3 min. Flow rate | |
| | | | 30 mL/min.) | |
| | | | Procedure M: crude material was purified | |
| | | | by NP chromatography (CombiFlash ® | |
| | | | Redisep column 24 g silica; eluent: solvent | |
| | | | A = cyclohexane; solvent B = ethyl acetate; | |
| | | | 0% B for 1 min followed by gradient 0%- | |
| | | | 60% B over 9 min. Flow rate 35 mL/min.) | |
| | | | Procedure N: crude material was purified | |
| | | | by NP chromatography (CombiFlash ® | |
| | | | Redisep column 24 g silica; eluent: solvent | |
| | | | A = cyclohexane; solvent B = ethyl acetate; | |
| | | | 10% B for 1 min followed by gradient 10%- | |
| | | | 70% B over 8 min. then held at 70% for 2 | |
| | | | min then gradient 70%-90% over 1.5 min. | |
| | | | Finally, held at 90% over 3.5 min. Flow | |
| | | | rate 35 mL/min.) | |

### Preparation of (E)-1-(3-((2-(aminomethyl)-3-fluoroallyl)sulfonyl)benzyl)azepan-2-one hydrochloride (Compound 36)

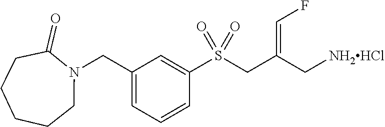

### Procedure X: Preparation of tert-butyl 6-((3-iodobenzyl)amino)hexanoate

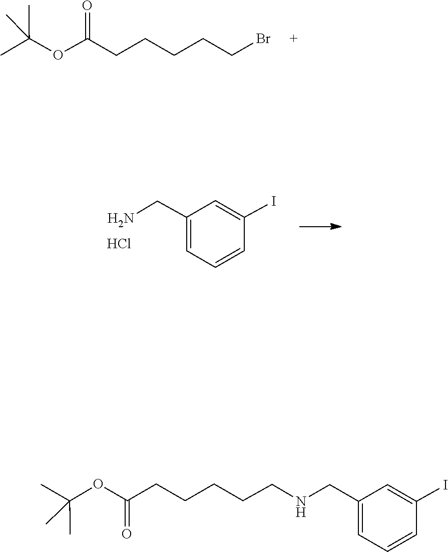 [SMILES: CC(C)(C)OC(=O)CCCCCBr ; Cl ; NCc1cccc(I)c1 ; CC(C)(C)OC(=O)CCCCCNCc1cccc(I)c1]

A mixture of tert-butyl 5-bromopentanoate (300 mg, 1.27 mmol) and (3-iodophenyl)methanamine hydrochloride (341 mg, 1.27 mmol) and potassium carbonate (525 mg, 3.80 mmol) in DMF (3.0 mL) was stirred at rt overnight. LCMS analysis showed completion of the reaction. Water (15 mL) was then added and the product was extracted with ethyl acetate. The combined organic extract was washed with brine, dried over Na₂SO₄ and then concentrated in vacuo. The crude product was purified by RP chromatography (Reveleris® C18 40 g; eluent: solvent A=12 mM aq HCl; solvent B=methanol; 20% B for 3 min followed by gradient 20-44% B over 5 min. Finally, held at 44% B for 3.5 min. Flow rate 40 mL/min.) to afford tert-butyl 6-((3-iodobenzyl)amino)hexanoate (0.30 g, 59%).

### Procedure Y: Preparation of 6-((3-iodobenzyl)amino)hexanoic acid

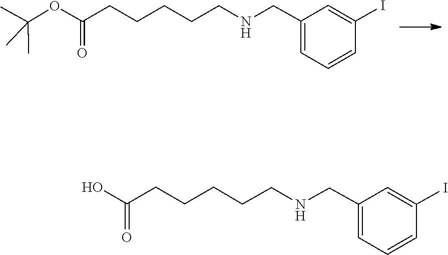 [SMILES: CC(C)(C)OC(=O)CCCCCNCc1cccc(I)c1 ; O=C(O)CCCCCNCc1cccc(I)c1]

To a solution of tert-butyl 6-((3-iodobenzyl)amino)hexanoate (30 mg, 0.74 mmol) in CH₂Cl₂ (3.0 mL) was added TFA (3.0 mL). The resulting mixture was stirred at rt for 2 h. LCMS analysis after this time showed completion of the reaction. Volatiles were then removed under vacuum. Ethyl acetate (5 mL) was added and the mixture was concentrated to remove residual TFA affording crude 6-((3-iodobenzyl)amino)hexanoic acid (300 mg, quant. yield). This material was used in the next step without further purification.

### Procedure Z: Preparation of 1-(3-iodobenzyl)azepan-2-one

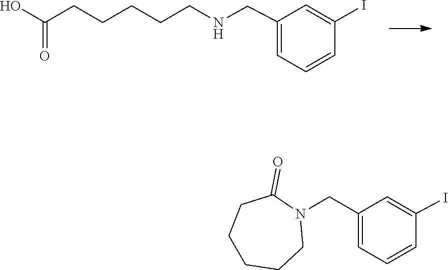 [SMILES: O=C(O)CCCCCNCc1cccc(I)c1 ; O=C1CCCCCN1Cc1cccc(I)c1]

To a stirring solution of 6-((3-iodobenzyl)amino)hexanoic acid (300 mg, 0.74 mmol) in DMF (3.0 mL) was added N,N-diisopropylethylamine (0.65 mL, 3.72 mmol) and HATU (424 mg, 1.12 mmol). The resulting mixture was stirred at rt for 2 h. LCMS analysis after this time showed completion of the reaction. The reaction mixture was diluted with water (25 mL). The product was extracted with ethyl acetate and the combined organic extract was washed with brine, dried over Na₂SO₄ and concentrated in vacuo. The crude product was purified by NP chromatography (CombiFlash® Redisep column 24 g silica; eluent: solvent A=cyclohexane; solvent B=ethyl acetate; 0% B for 1 min followed by gradient 0%-50% B over 13 min. then held at 50% B for 3 min. Flow rate 30 mL/min.) to afford 1-(3-iodobenzyl)azepan-2-one (0.15 g, 60%). ¹H NMR (300 MHz, CDCl₃) δ 7.63-7.57 (m, 2H), 7.24 (dt, J=7.6, 1.4 Hz, 1H), 7.10-7.02 (m, 1H), 4.53 (s, 2H), 3.34-3.25 (m, 2H), 2.61 (tt, J=4.2, 1.9 Hz, 2H), 1.72 (dt, J=7.8, 2.7 Hz, 4H), 1.52 (qd, J=5.8, 3.9, 3.1 Hz, 2H).

### Procedure AA: Preparation of tert-butyl (E)-(3-fluoro-2-(mercaptomethyl)allyl)carbamate

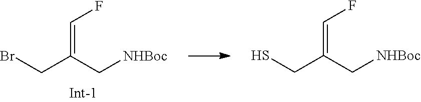 [SMILES: CC(C)(C)OC(=O)NC/C(=C\F)CBr ; CC(C)(C)OC(=O)NC/C(=C\F)CS]

A stirring mixture of Int-1 (540 mg, 2.01 mmol) and thiourea (169 mg, 2.22 mmol) in methanol (10 mL) was heated at reflux for 1.5 h. LCMS analysis after this time showed complete formation the intermediate S-alkyl isothiourea. Aqueous NaOH (2.0 M; 1.20 mL, 2.40 mmol) was then added and the reaction mixture was heated at reflux for 1 h. LCMS analysis showed absence of the isothiourea intermediate and formation of the desired product. The reaction mixture was concentrated under vacuum and the residue was adsorbed on silica gel. Normal-phase chromatography was performed (CombiFlash® Redisep column 24 g silica; eluent: solvent A=cyclohexane; solvent B=ethyl acetate; 10% B for 1 min followed by gradient 10%-50% B over 7 min. then held at 50% B for 2 min. Flow rate 30 mL/min.) to afford (170 mg, 19%) of the impure title compound. LC-MS analysis of the purified material indicated several impurities with an approximate purity of 50%. This material was progressed to the next step without further purification.

### Procedure AB: Preparation of tert-butyl (E)-(3-fluoro-2-(((3-((2-oxoazepan-1-yl)methyl)phenyl)thio)methyl)allyl)carbamate

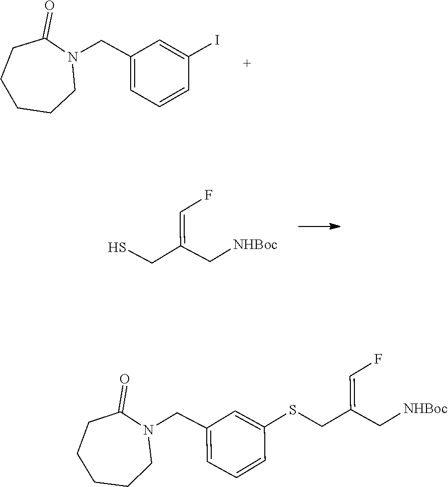 [SMILES: O=C1CCCCCN1Cc1cccc(I)c1 ; CC(C)(C)OC(=O)NC/C(=C\F)CS ; CC(C)(C)OC(=O)NC/C(=C\F)CSc1cccc(CN2CCCCCC2=O)c1]

A stirring mixture of 1-(3-iodobenzyl)azepan-2-one (150 mg, 0.46 mmol), tert-butyl (E)-(3-fluoro-2-(mercaptomethyl)allyl)carbamate (168 mg, 0.46 mmol) potassium hydroxide (45 mg, 0.68 mmol) and finely powdered cupric oxide (3.63 mg, 46 μmol) in DMSO (1.0 mL) was heated to 80 °C under nitrogen for 6 h. LCMS analysis after this time showed formation of the desired product. The reaction mixture was cooled to rt and then diluted with water (10 mL). The product was extracted with ethyl acetate (3×10 mL). The combined organic layer was washed with brine, dried over Na₂SO₄ and then concentrated in vacuo. The crude material was purified by NP chromatography (CombiFlash® Redisep column 24 g silica; eluent: solvent A=cyclohexane; solvent B=ethyl acetate; 10% B for 1 min followed by gradient 10%-75% B over 12 min. Flow rate 30 mL/min.) to afford tert-butyl (E)-(3-fluoro-2-(((3-((2-oxoazepan-1-yl)methyl)phenyl)thio)methyl)allyl)carbamate (40 mg, 21%).

### Procedure AC: Preparation of tert-butyl (E)-(3-fluoro-2-(((3-((2-oxoazepan-1-yl)methyl)phenyl)sulfonyl)methyl)allyl)carbamate

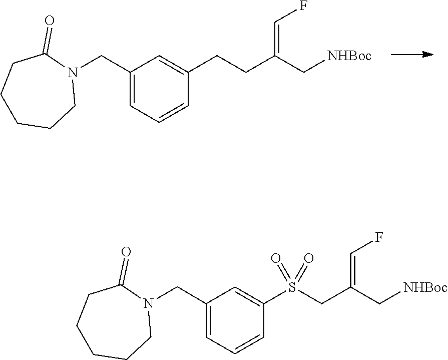 [SMILES: CC(C)(C)OC(=O)NC/C(=C\F)CCc1cccc(CN2CCCCCC2=O)c1 ; CC(C)(C)OC(=O)NC/C(=C\F)CS(=O)(=O)c1cccc(CN2CCCCCC2=O)c1]

To a stirring solution of tert-butyl (E)-(3-fluoro-2-(((3-((2-oxoazepan-1-yl)methyl)phenyl)thio)-methyl)allyl)carbamate (40 mg, 0.09 mmol) in methanol (2.0 mL) was added hydrogen peroxide (30% w/v, 1.0 mL) followed by a solution of sodium tungstate dihydrate (31 mg, 0.09 mmol) in water (0.5 mL). The resulting mixture was then stirred at rt for 2 h. LCMS analysis after this time showed complete absence of starting material. Water (10 mL) was added and the product was extracted with ethyl acetate. The combined organic extract was washed with brine, dried over Na₂SO₄ and concentrated in vacuo to afford tert-butyl (E)-(3-fluoro-2-(((3-((2-oxoazepan-1-yl)methyl)phenyl)sulfonyl)methyl)allyl)carbamate (20 mg, 46%). This material was progressed to the final step without purification.

### Procedure AD: Preparation of (E)-1-(3-((2-(aminomethyl)-3-fluoroallyl)sulfonyl)benzyl)azepan-2-one hydrochloride (Compound 36)

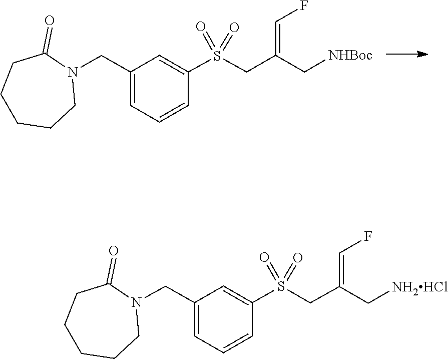

(E)-(3-fluoro-2-(((3-((2-oxoazepan-1-yl)methyl)phenyl)sulfonyl)methyl)allyl)carbamate (20 mg, 0.04 mmol) was added to a solution of HCl (4.0 M in dioxane; 1.0 mL). The resulting mixture was stirred at rt for 2 h. LC-MS analysis after this time indicated complete consumption of starting material. The reaction mixture was then concentrated under vacuum. Ethyl acetate (2 mL) was added and the mixture was stirred for 5 min. The mixture, now containing precipitated product, was transferred to a vial and the solid was spun down in a centrifuge (4000 rpm, 4 min). The liquid was carefully decanted and the solid residue was dried at 60 °C for 1 h to afford Compound 36 (2.5 mg, 15%). ¹H NMR (300 MHz, Methanol-d₄) δ 7.88 (d, J=8.3 Hz, 2H), 7.69 (dt, J=14.9, 7.5 Hz, 2H), 6.77 (d, J=80.4 Hz, 1H), 4.71 (s, 2H), 4.10 (d, J=2.9 Hz, 2H), 3.85 (d, J=2.3 Hz, 2H), 3.54-3.44 (m, 2H), 2.72-2.59 (m, 2H), 1.77 (d, J=10.9 Hz, 4H), 1.58 (s, 2H). LC-MS: m/z 377 [M+Na]+; RT=3.00 min (Method A).

### Preparation of (Z)-3-fluoro-2-((phenylsulfonyl)methyl)prop-2-en-1-amine hydrochloride (Compound 37)

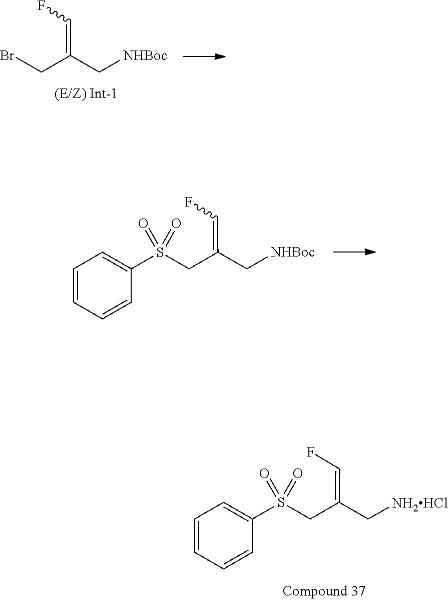

Compound 37 was prepared by a two-step process starting from an E/Z mixture of intermediate 1 ((E/Z) Int-1) employing chemistry analogous to that described herein for the synthesis of Compound 1. The final compound (37) was obtained in pure isomeric form through application of HPLC separation. The detailed synthesis of (E/Z) Int-1 has been disclosed in WO2013/163675.

Compound 37: ¹H NMR (400 MHz, Methanol-d₄) δ 7.96 (dd, J=8.0, 0.8 Hz, 2H), 7.75 (dt, J=7.2, 0.8 Hz, 1H), 7.65 (dd, J=8.4, 7.6 Hz, 2H), 7.06 (d, J=79.6 Hz, 1H), 4.22 (d, J=2.0 Hz, 2H), 3.76 (d, J=2.8 Hz, 2H).

### Example 2

Method to Determine the Ability of Compound 1 of the Current Invention to Inhibit LOX and LOXL1-4 from Different Sources

LOX and LOXL protein family members can be acquired as recombinant active proteins from commercial sources, or extracted from animal tissues like bovine aorta, tendons, pig skin; or prepared from cell cultures. The inhibitory effects of the compounds of the present invention were tested against the given LOX or LOXL preparation using a method based on the detection of hydrogen peroxide with an Amplex Red oxidation assay [Zhou et al, 1997]. The assay was developed using either 384 or 96 well format. Briefly, in a standard black, clear bottom 384 well plate assay 25 L of a dilution of any of the isoenzymes and orthologues in 1.2 M urea, 50 mM sodium borate buffer (pH 8.2) were added into each well in the presence of 1 M mofegiline and 0.5 mM pargyline (to inhibit SSAO and MAO-B and MAO-A, respectively; not necessary if the enzyme is from a recombinant or purified form). Test compounds were dissolved in DMSO and tested in a Concentration Response Curve (CRC) with 11 data points, typically in the micromolar or nanomolar range after incubation with the enzyme for 30 min at 37 °C. 25 L of a reaction mixture containing twice the K_{M} concentration of putrescine (Sigma Aldrich, e.g. 20 mM for LOX and LOXL1 or 10 mM for LOXL2 and 4 mM for LOXL3 and LOXL4), 120 M Amplex Red (Thermo Fisher Scientific) and 1.5 U/mL horseradish peroxidase (Sigma Aldrich) prepared in 1.2 M urea, 50 mM sodium borate buffer (pH 8.2) were then added to the corresponding wells. The above volumes were doubled in the case of 96 wells plate. The fluorescence (RFU) was read every 2.5 min for 30 min at 37 °C, excitation 544 nm and emission 590 (Optima; BMG labtech). The slope/min of the kinetics for each well was calculated using MARS data analysis software (BMG labtech) and this value was used to deduce the IC₅₀ value (Dotmatics). The ability of the inventive compounds to inhibit the amine oxidase activity of LOX and other family members is shown in Table 5. Bovine LOX was used as a surrogate of human recombinant LOX due to its analogous pharmacological properties and the poor and unreliable behaviour often displayed by human recombinant LOX obtained commercially or otherwise. Table 6 shows corresponding results obtained after incubation with the enzyme for 2 h at 37 °C.

TABLE 5
LOX, LOXL1-4 inhibitory activities of Compound 1

| | | Human | Human | Human | Human |
|---|---|---|---|---|---|
| | Bovine | recombinant | recombinant | recombinant | recombinant |
| | LOX | LOXL1 | LOXL2 | LOXL3 | LOXL4 |
| | Activity | Activity | Activity | Activity | Activity |
| Compound | IC₅₀ (μM) | IC₅₀ (μM) | IC₅₀ (μM) | IC₅₀ (μM) | IC₅₀ (μM) |
| BAPN | 2.3 | 2.5 | 0.4 | 0.5 | 0.3 |
| 1 | 10 | 13.2 | 4.8 | 5.9 | 4.7 |

TABLE 6
LOX and LOXL2 Inhibitory activities of
examples of compounds of the invention

| | Bovine LOX | Human LOXL2 |
|---|---|---|
| Compound | Activity IC₅₀ (μM) | Activity IC₅₀ (μM) |
| 1 | B | A |
| 2 | A | A |
| 3 | B | A |
| 4 | B | A |
| 5 | A | A |
| 6 | B | B |
| 7 | C | A |
| 8 | A | A |
| 9 | A | A |
| 10 | A | A |
| 11 | A | A |
| 12 | B | B |
| 13 | A | A |
| 14 | A | A |
| 15 | A | A |
| 16 | A | A |
| 17 | A | A |
| 18 | A | A |
| 19 | A | A |
| 20 | A | A |
| 21 | A | A |
| 22 | A | A |
| 23 | A | A |
| 24 | A | A |
| 25 | A | A |
| 26 | C | B |
| 27 | A | A |
| 28 | B | A |
| 29 | A | A |
| 30 | B | B |
| 31 | C | C |
| 32 | A | A |
| 33 | C | C |
| 34 | B | — |
| 35 | B | A |
| 36 | C | B |
| 37 | A | A |

IC₅₀: A = <2 μM, B = <5 μM, C = <10 μM, D = >10 μM

### Example 3

### Compounds of the Current Invention Exhibit Sustained Inhibition of LOXL1 and LOXL2

For meaningful pharmacological effect in the presence of high substrate concentration, compounds that exert sustained, long lasting inhibition of LOX and LOXL1-4 present an advantage over competitive inhibitors as the pharmacological effect may outlast the presence of the unbound inhibitor. In a preferred embodiment compounds in the current invention exhibit sustained inhibition of LOX and LOXL1-4.

### Method of Determining Sustained Inhibition of LOX and LOXL1-4 by Compound 1

Jump Dilution experiment: The assay was developed using a 96 well format and the starting enzyme concentration was set 100 times higher than for the inhibition studies. The enzyme was incubated for 40 minutes at 37 °C in presence of 10×IC₅₀ concentrations of the test inhibitor. After the incubation, the mixture was diluted 50x in assay buffer, followed by a further 2× dilution in Amplex Red-horseradish peroxidase-putrescine reaction mix (as for Example 2) prior to the fluorescence measurement. Results were expressed in % recovery of the signal at 21-30 min by comparison with non-inhibited controls. LOXL1 is used as a surrogate for LOX owing to similar pharmacological behaviour.

A reversible standard, used as a control, showed quasi-complete activity recovery after 30 min, for both LOXL1 and LOXL2 (88.1% and 89.7% respectively). In contrast, BAPN, a known pan LOX irreversible inhibitor and Compound 1 exhibit sustained inhibition of LOXL1 and LOXL2, with the LOXL1/LOXL2 enzyme recovering only 0.3%/11.2% for BAPN and 2.5%/2.5% for Compound 1, at 21-30 min.

### Example 4

### Method to Determine the Ability of Compound 1 to Inhibit Human Recombinant SSAO/VAP-1

Human recombinant SSAO/VAP-1 amine oxidase activity was determined using the coupled colorimetric method as described for monoamine oxidase, copper-containing amine oxidases and related enzymes [Holt A. and Palcic M., 2006]. Briefly, a cloned cDNA template corresponding to residues 34-763 of human SSAO/VAP-1, and incorporating a mouse Ig kappa (κ) signal sequence, N-terminal flag epitope tag and tobacco etch virus (TEV) cleavage site, was assembled in a mammalian expression vector (pLO-CMV) by Geneart AG. This vector containing human SSAO/VAP-1 residues was transfected into CHO-K1 glycosylation mutant cell line, Lee 8. A clone stably expressing human SSAO/VAP-1 was isolated and cultured in large scale. Active human SSAO/VAP-1 was purified and recovered using immunoaffinity chromatography. This was used as the source for SSAO/VAP-1 activity. A high-throughput fluorescent assay was developed using either 96 or 384 well format. Briefly, in a standard 384 well plate assay 25 μL of purified human SSAO/VAP-1 (0.25 μg/mL) in 0.1 M sodium phophate buffer (pH 7.4) was added into each well. Test compounds were dissolved in DMSO and tested in a Concentration Response Curve (CRC) with 4-11 data points, typically in the micromolar or nanomolar range after incubation with human SSAO/VAP-1 for 30 min at 37 °C. After 30 min incubation, 25 μL of the reaction mixture containing 600 μM benzylamine (Sigma Aldrich), 120 μM Amplex Red (Thermo Fisher Scientific) and 1.5 U/mL horseradish peroxidase (Sigma Aldrich) prepared in 0.1 M sodium phosphate buffer (pH 7.4) were added to the corresponding well. The fluorescence unit (RFU) was read every 2.5 min for 30 min at 37 °C excitation 544 nm and emission 590 (Optima; BMG labtech). The slope/min of the kinetics for each well was calculated using MARS data analysis software (BMG labtech) and this value was used to deduce the IC₅₀ value (Dotmatics). The ability of Compound 1 to inhibit SSAO/VAP-1 is shown in Table 7.

### Example 5

### Method to Determine the Ability of Compound 1 to Inhibit Human Recombinant MAO-B

The specificity of Compound 1 of the current invention was tested by determining their ability to inhibit MAO-B activity in vitro using recombinant human MAO-B (0.02 mg/mL; Sigma Aldrich). The assay was performed in a similar way as for human SSAO/VAP-1 (Example 4) except, the substrate benzylamine was used at 100 μM. The ability of Compound 1 to inhibit MAO-B is shown in Table 7.

### Example 6

### Method to Determine the Ability of Compound 1 to Inhibit Human Recombinant MAO-A

The specificity of Compound 1 of the current invention was tested by determining their ability to inhibit MAO-A activity in vitro, using recombinant human MAO-A (0.003 mg/mL; Sigma Aldrich). The assay was performed in a similar way as for human SSAO/VAP-1 (Example 4) except, the incubation with the test compounds was extended to 2 hours, and the substrate tyramine was used at 200 μM in place of benzylamine. The ability of Compound 1 to inhibit MAO-A is shown in Table 7.

### Example 7

### Method to Determine the Ability of Compound 1 to Inhibit Human Recombinant DAO

The specificity of Compound 1 of the current invention was tested by determining their ability to inhibit DAO activity in vitro, using recombinant human DAO (100 ng/mL; kindly provided by Prof. Boehm, Department of Clinical Pharmacology, Medical University, Vienna). The assay was performed in a similar way as for human SSAO/VAP-1 (Example 4) except, the incubation with the test compounds was extended to 2 hours, and the substrate putrescine was used at 200 μM in place of benzylamine. The ability of Compound 1 to inhibit DAO is shown in Table 7.

LOX and LOXL1-4 enzymes are members of a large family of flavin-dependent and copper-dependent amine oxidases, which includes SSAO/VAP-1, monoamine oxidase-B (MAO-B), monoamine oxidase-A (MAO-A) and diamine oxidase (DAO). Compound 1 of the present invention selectively inhibit members of the LOX family of enzymes with respect to SSAO/VAP-1, MAO-B, MAO-A and DAO. Examples of the magnitude of selectivity can be seen in Table 7.

TABLE 7
SSAO/VAP-1, MAO-B, MAO-A and DAO inhibitory
activities of Compound 1.

| | Human | | | |
|---|---|---|---|---|
| | recombinant | Human | Human | Human |
| | SSAO/VAP-1 | MAO-B | MAO-A | DAO |
| | Activity | Activity | Activity | Activity |
| Compound | IC50 (uM) | IC50 (uM) | IC50 (uM) | IC50 (uM) |
| 1 | >30 | >30 | >100 | >100 |

### Example 8

### Measurement of the Time-Dependent Inhibition of LOXL1 and LOXL2 by Compound 1

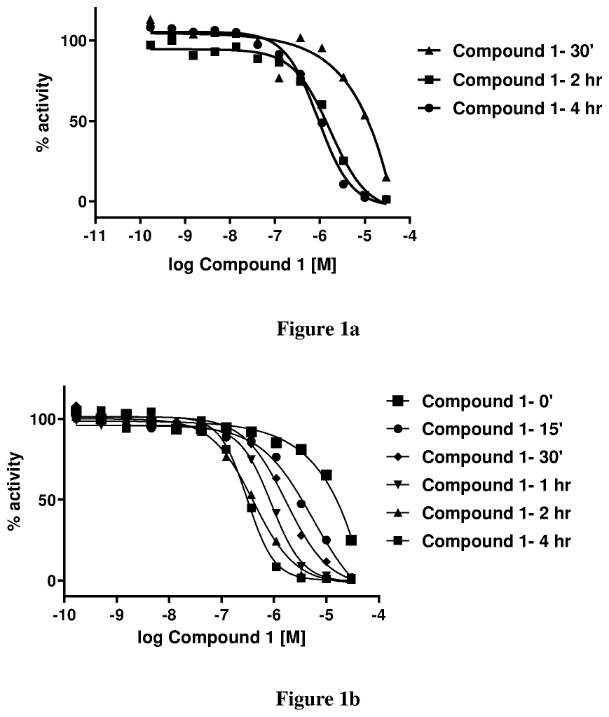

The time dependent inhibitory effects of the Compound 1 of the present invention were tested against the given LOXL1 preparation and LOXL2 preparation using a method based on the detection of hydrogen peroxide with an Amplex Red oxidation assay [Zhou et al. 1997]. The assay was developed for use in either 384 or 96 well format. Briefly, in a standard black, clear bottom 384 well plate assay 25 L of a dilution of any of the isoenzymes and orthologues in 1.2 M urea, 50 mM sodium borate buffer (pH 8.2) were added into each well in the presence of 1 M mofegiline and 0.5 mM pargyline (to inhibit SSAO and MAO-B and MAO-A, respectively; not necessary if the enzyme is from a recombinant or purified form). Test compound (Compound 1) was dissolved in DMSO and tested in a Concentration Response Curve (CRC) with 11 data points, typically in the micromolar or nanomolar range after incubation with the enzyme for different time points at 37 °C. 25 μL of a reaction mixture containing twice the K_{M} concentration of putrescine (Sigma Aldrich, e.g. 20 mM for LOXL1 or 10 mM for LOXL2), 120 M Amplex Red (Thermo Fisher Scientific) and 1.5 U/mL horseradish peroxidase (Sigma Aldrich) prepared in 1.2 M urea, 50 mM sodium borate buffer (pH 8.2) were then added to the corresponding wells. The fluorescence (RFU) was read every 2.5 min for 30 min at 37 °C, excitation 544 nm and emission 590 nm (Optima; BMG labtech). The slope/min of the kinetics for each well was calculated using MARS data analysis software (BMG labtech) and this value was used to deduce the IC₅₀ value (Dotmatics). FIG. 1a/b and Table 8a/b demonstrate that Compound 1 exhibits time dependent LOXL1 and LOXL2 inhibition, with the apparent potency increasing with time.

TABLE 8a
Time-dependent inhibition of LOXL1 by Compound 1.

| | IC₅₀ | IC₅₀ | IC₅₀ |
|---|---|---|---|
| | (μM) | (μM) | (μM) |
| Compound | 4 h | 2 h | 30′ |
| 1 | 0.92 | 1.70 | 9.47 |

TABLE 8b
Time-dependent inhibition of LOXL2 by Compound 1.

| | IC₅₀ | IC₅₀ | IC₅₀ | IC₅₀ | IC₅₀ | IC₅₀ |
|---|---|---|---|---|---|---|
| | (μM) | (μM) | (μM) | (μM) | (μM) | (μM) |
| Compound | 4 h | 2 h | 1 h | 30′ | 15′ | 0′ |
| 1 | 0.31 | 0.41 | 0.89 | 1.71 | 5.58 | 14.66 |

### Example 9

The following examples in Table 9 are intended to serve to illustrate topical formulations and should not be construed as limiting the generality of the disclosure of the description throughout this specification. It should be understood that numerous variations and modifications may be made while remaining within the scope of the invention.

TABLE 9
Topical Formulation

| Entry | Ingredient | Supplier Info. | Quantity (g) |
|---|---|---|---|
| 1 | Propylene glycol | Sydney Solvents | 150 |
| 2 | Sodium phosphate | Sigma-Aldrich; Cat. | 5 |
| | monobasic, anhydrous | No. S8282 | |
| 3 | Cetomacrogol 1000 | Croda: SP | 25 |
| | BPC flakes | Cetomacrogol 1000 | |
| | | MBAL-PA-(SG) | |
| 4 | Petroleum jelly | PCCA: Petrolatum | 120 |
| | | White USP | |
| 5 | Cetostearyl alcohol | C1618 Thai Alcohols | 75 |
| 6 | Millipore water | | 600 |

### Preparation of Topical Formulation

To a 1 L glass beaker containing 120 g of petroleum jelly was added 75 g of cetostearyl alcohol. The mixture was then warmed to 70 °C in a heat bath. Stirred well until all ingredients were dissolved to obtain the oil phase. Placed 5 g of sodium phosphate monobasic (NaH₂PO₄) into a 1 L glass bottle. Added 600 g of Millipore water. Stirred well until all solids were dissolved to obtain a sodium phosphate buffer. Placed 383.0 g of sodium phosphate buffer in a 500 mL beaker added test compound and adjusted the pH of the formulation. Heated to 70 °C in a heat bath. 150 g of propylene glycol was added in a 500 mL glass bottle, 25 g of cetomacrogol 1000 BPC flakes was added and the mixture was heated to 70 °C in a heat bath with stirring until a homogenous solution was achieved. This solution was combined with the sodium phosphate buffer. Stirred well until all ingredients were dissolved to obtain the aqueous phase. Removed oil phase and aqueous phase from the heat bath. Transferred aqueous phase to an aluminum mixer bowl and added oil phase while mixing. Homogenized while gradually cooling down to 25-30 °C to obtain the cream formulation. For preparation of cream formulation containing 0.3%, 1.0%, 1.5%, 2.0% and 4.0% Compound 1, the appropriate quantity of Compound 1 was dissolved in the sodium phosphate buffer prior to combining with the oil phase.

### Example 10

The stability of a drug product formulation can have a significant impact on the length and cost of drug development, the nature of the studies required to support regulatory submissions, and, ultimately, safety and approvability. It is important to minimize the amount of impurities or degradation products that form over time due to interactions between the various ingredients in a formulation. This can be particularly important in compositions that are designed to increase skin permeability.

Compound 1 exhibits good stability in the topical formulation 1, with the compound peak percentage above 99.5% at all tested time points, as shown in Table 10.

TABLE 10
Purity profile in the reference sample and in
samples stored at room temperature (rt) for 9 months.
% Purity of Compound 1 in topical
formulation containing 1% Compound 1

| t = 0 | t = 3 months | t = 6 months | t = 9 months |
|---|---|---|---|
| 99.83 | 99.81 | 99.82 | 99.79 |

### Example 11

Genotoxicity is a term that describes the properties of chemicals pertaining to their ability to cause damage to genetic material. This can lead to mutations and, possibly, various types of cancer. Evaluation of the genotoxicity effects of various materials for use as drug components is vital as it can directly impact drug developability and utility.

### Bacterial Reverse Mutation Test

The test item Compound 1 was tested for its mutagenic potential in the bacterial reverse mutation assay. The study was conducted using TA98, TA100, TA1535 and TA1537 strains of Salmonella typhimurium and WP2uvrA (pKM101) strain of Escherichia coli in three phases; a preliminary toxicity test, an initial mutation assay and a confirmatory mutation assay. The bacterial tester strains were exposed to the test item in the presence and absence of metabolic activation system (S9 fraction prepared from Aroclor 1254 induced rat liver).

The test item was soluble in DMSO at 50 mg/mL and was used as the vehicle of choice in the mutation assay. The test item was found to be stable in the vehicle DMSO for 6 h at room temperature and stable for 4 days at refrigerated temperature at 20 μg/mL and 50000 μg/mL

In the preliminary toxicity test, for selection of test doses for the mutation assay, Salmonella typhimurium, TA100 was exposed to the test item at 39.0625, 78.125, 156.25, 312.5, 625, 1250, 2500 and 5000 μg/plate along with a DMSO control using direct plate incorporation.

The results of this preliminary toxicity test showed that the test item did not precipitate on the basal agar plates up to 5000 μg/plate either in the presence, or the absence of metabolic activation.

The test item did not exhibit toxicity to the tester strain at any of the tested doses as the intensity of the bacterial background lawn as well as the mean revertant colony counts, was comparable to the DMSO control in both presence and absence of metabolic activation. Based on these observations, the OECD 471 recommended top dose of 5000 μg/plate was tested in the mutation assay.

The bacterial tester strains were exposed to the test item in triplicate at 50, 158, 500, 1581 and 5000 μg/plate. The initial mutation assay was conducted using the direct plate incorporation mode of exposure whereas the confirmatory mutation assay was carried out using the pre-incubation mode of exposure. The vehicle control (DMSO) and the appropriate positive controls were tested simultaneously. The mean and standard deviation of numbers of revertant colonies were calculated for each test dose and the controls for all the tester strains.

The results of the study from both the initial and confirmatory mutation assay showed that the test item did not show any positive mutagenic increase at any of the tested doses either in the presence or in the absence of metabolic activation in all the tester strains when compared to the respective vehicle control plates.

The results of the concentration analysis of the dose formulation sample of initial and confirmatory mutation assay confirmed that the top dose level of 5000 μg/plate was achieved which support the validity of the study conclusion.

Under identical test conditions, more than 3-fold increase in the mean numbers of revertant colonies in the positive controls were observed, demonstrating the sensitivity of the assay procedure used.

The study indicated that Compound 1 was not mutagenic in this Bacterial Reverse Mutation Assay up to the top dose 5000 μg/plate, under the conditions of testing employed.

### Example 12

### Compound 1 Exhibits Good Diffusion Through Human Skin

Skin permeability is essential when considering topical application to treat skin conditions. One way to evaluate skin permeability is to use a Franz diffusion cell system with human skin as a membrane (Particle Sciences; Technical Brief 2009; Vol. 10). The donor compound (Compound 1), topically formulated, is applied on the top chamber and diffusion through the skin is monitored at relevant time points by sampling the solution in the receptor chamber via the sampling port. The concentration of Compound 1 was determined by quantitative HPLC analysis.

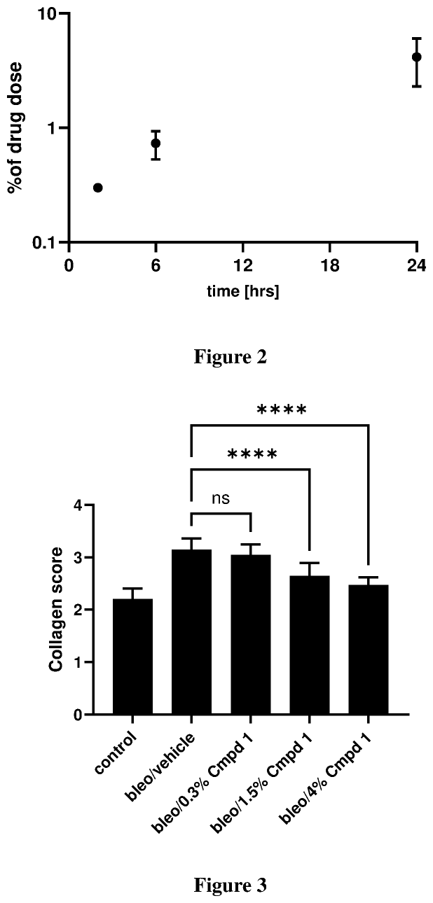

Compound 1 displays good permeability and diffusion (from a topical formulation containing 2% Compound 1) across the membrane, in a time dependent manner, to reach high levels in the receptor chamber after 24 hours (FIG. 2).

### Example 13

### Mouse Model of Sclerosis

Subcutaneous bleomycin (0.1 U/kg) was administered every second day (for 21 days total) to male C₅₇BL/6 to induce skin fibrosis as a model of sclerosis. The lesions were treated, from day 3, with either Vehicle or various concentrations of Compound 1 in a topical formulation. Histology was completed after 21 days. The histological analysis is shown in FIG. 3 and shows significant improvement in the average collagen score.

### Example 14

### Rodent Injury Model

Mice received injury by excision of full-thickness skin (3 cm² on the flank of the mice). In the treatment groups, 4% Compound 1 solution was applied topically, once a day, starting from 24 hours post injury to 1 week post injury. Wounds were then left to heal for an additional week. Mice were euthanized at 4-6 weeks post injury and the tissue was analyzed for collagen content, hydroxyproline as a surrogate marker of collagen content, extracted cross-link biomarkers: HLNL, DHLNL (reduced forms of deH-HLNL and deH-DHLNL respectively), PYD and DPD, elastin content, and changes in gross morphology and histology (using polarised light microscopy, immunohistochemistry, standard staining markers and LCMSMS).

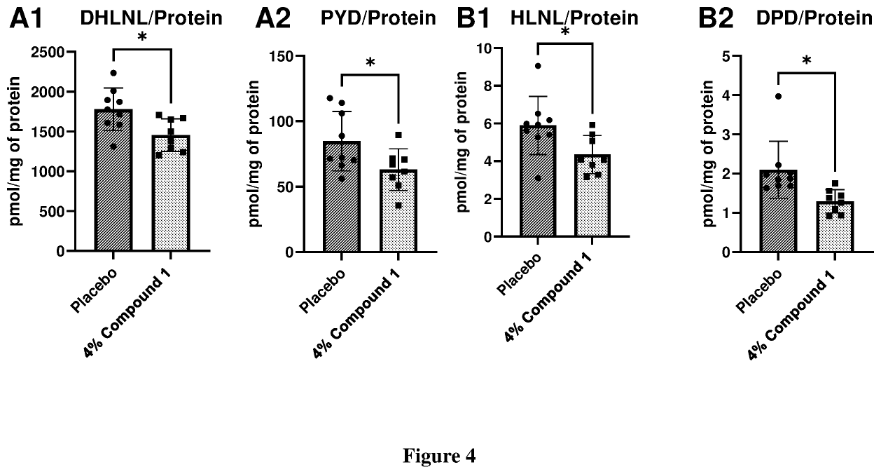

Immature and mature crosslinks (measured using LCMS and normalized for protein content) was reduced in treated tissue compared to control (FIG. 4).

### REFERENCES

Holt, A., Palcic, M. M., (2006). A peroxidase-coupled continuous absorbance plate-reader assay for Flavin monoamine oxidases, copper-containing amine oxidases and related enzymes. Nat Protoc, 1(5), 2498-2505
Zhou et al. (1997). A stable nonfluorescent derivative of resorufin for the fluorometric determination of trace hydrogen peroxide: applications in detecting the activity of phagocyte NADPH oxidase and other oxidases. Anal. Biochem, 253, 162-168.

1. A compound of Formula I: 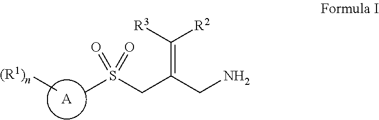
or a pharmaceutically acceptable salt thereof; wherein:
A is aryl;
each R¹ is independently selected from the group consisting of X—R⁴, halogen, deuterium, C₁₋₆alkyl, O—C₁₋₆alkyl, aryl, heteroaryl, cycloalkyl, heterocycloalkyl, —CN, —NO₂, —C(O)OR⁵, —C(O)NR⁶R⁷, —S(O)₂NR⁶R⁷, —S(O)₂R⁸, —NR⁹C(O)R¹⁰, and —NR⁹S(O)₂R¹⁰; wherein each C₁₋₆alkyl, aryl, heteroaryl, cycloalkyl and heterocycloalkyl is optionally substituted by one or more substituents selected from the group consisting of halogen, —OH, and —C₁₋₄alkyl;
R² is H or F;
R³ is H or F; provided that one, and only one, of R² or R³ is F;
X is selected from the group consisting of O, CH₂, and S(O)₂;
R⁴ is selected from the group consisting of aryl and heterocycloalkyl; wherein each aryl is optionally substituted by —S(O)₂R⁸; and wherein each heterocycloalkyl is optionally substituted by ═O;
R⁵ is selected from the group consisting of hydrogen and C₁₋₆alkyl;
R⁶ and R⁷ are independently selected from the group consisting of hydrogen and C₁₋₆alkyl;
R⁸ is C₁₋₆alkyl;
R⁹ is hydrogen or C₁₋₆alkyl;
R¹⁰ is C₁₋₆alkyl; and
n is 0, 1, 2, 3, 4 or 5.

2. The compound according to claim 1, wherein R² is F and R³ is H.

3. The compound according to claim 1, wherein A is selected from the group consisting of phenyl, naphthyl and 1,4-benzodioxanyl.

4. The compound according to claim 1, wherein each R¹ is independently selected from the group consisting of X—R⁴, halogen, deuterium, C₁₋₆alkyl, O—C₁₋₆alkyl, heteroaryl, —CN, —NO₂, —C(O)OR⁵, —C(O)NR⁶R⁷ and —S(O)₂R⁸; wherein each C₁₋₆alkyl and heteroaryl is optionally substituted by one or more substituents selected from the group consisting of halogen and —C₁₋₄alkyl.

5. The compound according to claim 1 of Formula Ib 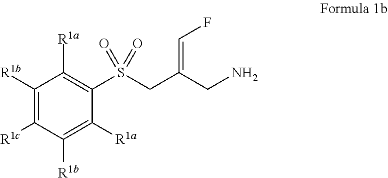
or a pharmaceutically acceptable salt thereof; wherein:
each R^{1a} is independently selected from the group consisting of X—R⁴, halogen, deuterium, C₁₋₄alkyl and —CN;
each R^{1b} is independently selected from the group consisting of X—R⁴, halogen, deuterium, —C₁₋₄alkyl, —O—C₁₋₄alkyl, heteroaryl, —CN, —NO₂, —C(O)OR⁵, —C(O)NR⁶R⁷, —S(O)₂R^{B}, wherein each —C₁₋₄alkyl and heteroaryl is optionally substituted by one or more substituents selected from the group consisting of halogen, and —C₁₋₄alkyl;
R¹⁰ is selected from the group consisting of deuterium, —C₁₋₄alkyl, —CN, —C(O)OR⁵ and —S(O)₂R⁸;
X is selected from the group consisting of O, CH₂, and S(O)₂;
R⁴ is selected from the group consisting of aryl and heterocycloalkyl; wherein each aryl is optionally substituted by —S(O)₂R⁸; and wherein each heterocycloalkyl is optionally substituted by ═O;
R⁵ is selected from the group consisting of hydrogen and C₁₋₄alkyl;
R⁶ and R⁷ are independently selected from the group consisting of hydrogen and C₁₋₄alkyl; and
R⁸ is C₁₋₄alkyl.

6. The compound according to claim 5, wherein
each R^{1a} is independently selected from the group consisting of X—R⁴, chlorine, deuterium, methyl and —CN;
each R^{1b} is independently selected from the group consisting of X—R⁴, halogen, deuterium, —C₁₋₄alkyl, —OCH₃, heteroaryl, —CN, —NO₂, —C(O)OH, —C(O)NH₂, —S(O)₂CH₃, wherein each —C₁₋₄alkyl and heteroaryl is optionally substituted by one or more substituents selected from the group consisting of fluorine, and —C₁₋₄alkyl;
R^{1c} is selected from the group consisting of deuterium, methyl, —CN, —C(O)OH and —S(O)₂CH₃;
X is selected from the group consisting of O, CH₂, and S(O)₂; and
R⁴ is selected from the group consisting of phenyl and 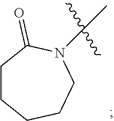 [SMILES: *N1CCCCCC1=O]
wherein each phenyl is optionally substituted by —S(O)₂CH₃.

7. The compound according to claim 5, wherein
each R^{1b} is independently selected from the group consisting of X—R⁴, F, Cl, Br, deuterium, -methyl, —CF₃, isopropyl, —OCH₃, thiazolyl, pyrazolyl, —CN, —NO₂, —C(O)OH, —C(O)NH₂, —S(O)₂CH₃, wherein each thiazolyl or pyrazolyl is optionally substituted by methyl.

8. The compound according to claim 1 selected from the group consisting of

| | | |
|---|---|---|
| 1 | 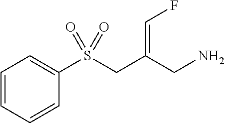 [SMILES: NC/C(=C\F)CS(=O)(=O)c1ccccc1] | (E)-3-fluoro-2- ((phenylsulfonyl)methyl)prop-2-en-1- amine |
| 2 | 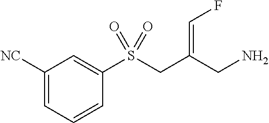 [SMILES: N#Cc1cccc(S(=O)(=O)C/C(=C/F)CN)c1] | (E)-3-((2-(aminomethyl)-3- fluoroallyl)sulfonyl)benzonitrile |
| 3 | 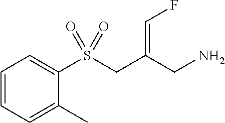 [SMILES: Cc1ccccc1S(=O)(=O)C/C(=C/F)CN] | (E)-3-fluoro-2-((o- tolylsulfonyl)methyl)prop-2-en-1-amine |
| 4 | 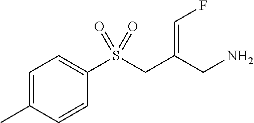 [SMILES: Cc1ccc(S(=O)(=O)C/C(=C/F)CN)cc1] | (E)-3-fluoro-2-(tosylmethyl)prop-2-en-1- amine |
| 5 | 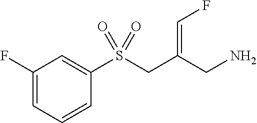 [SMILES: NC/C(=C\F)CS(=O)(=O)c1cccc(F)c1] | (E)-3-fluoro-2-(((3- fluorophenyl)sulfonyl)methyl)prop-2-en- 1-amine |
| 6 | 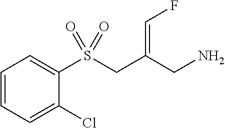 [SMILES: NC/C(=C\F)CS(=O)(=O)c1ccccc1Cl] | (E)-2-(((2-chlorophenyl)sulfonyl)methyl)- 3-fluoroprop-2-en-1-amine |
| 8 | 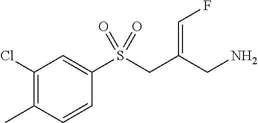 [SMILES: Cc1ccc(S(=O)(=O)C/C(=C/F)CN)cc1Cl] | (E)-2-(((3-chloro-4- methylphenyl)sulfonyl)methyl)-3- fluoroprop-2-en-1-amine |
| 9 | 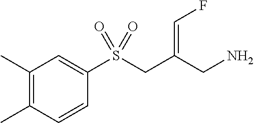 [SMILES: Cc1ccc(S(=O)(=O)C/C(=C/F)CN)cc1C] | (E)-2-(((3,4- dimethylphenyl)sulfonyl)methyl)-3- fluoroprop-2-en-1-amine |
| 10 | 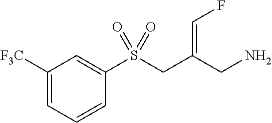 [SMILES: NC/C(=C\F)CS(=O)(=O)c1cccc(C(F)(F)F)c1] | (E)-3-fluoro-2-(((3- (trifluoromethyl)phenyl)sulfonyl)methyl) prop-2-en-1-amine |
| 11 | 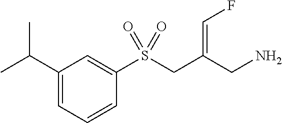 [SMILES: CC(C)c1cccc(S(=O)(=O)C/C(=C/F)CN)c1] | (E)-3-fluoro-2-(((3- isopropylphenyl)sulfonyl)methyl)prop-2- en-1-amine |
| 12 | 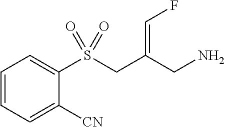 [SMILES: N#Cc1ccccc1S(=O)(=O)C/C(=C/F)CN] | (E)-2-((2-(aminomethyl)-3- fluoroallyl)sulfonyl)benzonitrile |
| 13 | 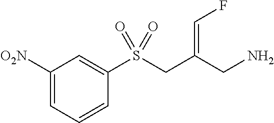 [SMILES: NC/C(=C\F)CS(=O)(=O)c1cccc([N+](=O)[O-])c1] | (E)-3-fluoro-2-(((3- nitrophenyl)sulfonyl)methyl)prop-2-en-1- amine |
| 14 | 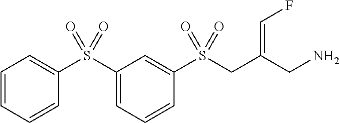 [SMILES: NC/C(=C\F)CS(=O)(=O)c1cccc(S(=O)(=O)c2ccccc2)c1] | (E)-3-fluoro-2-(((3- (phenylsulfonyl)phenyl)sulfonyl)methyl) prop-2-en-1-amine |
| 15 | 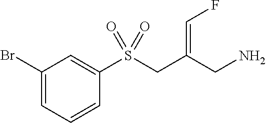 [SMILES: NC/C(=C\F)CS(=O)(=O)c1cccc(Br)c1] | (E)-2-(((3- bromophenyl)sulfonyl)methyl)-3- fluoroprop-2-en-1-amine |
| 16 | 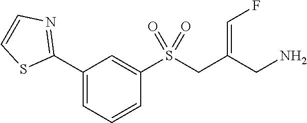 [SMILES: NC/C(=C\F)CS(=O)(=O)c1cccc(-c2nccs2)c1] | (E)-3-fluoro-2-(((3-(thiazol-2- yl)phenyl)sulfonyl)methyl)prop-2-en-1- amine |
| 17 | 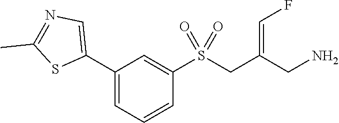 [SMILES: Cc1ncc(-c2cccc(S(=O)(=O)C/C(=C/F)CN)c2)s1] | (E)-3-fluoro-2-(((3-(2-methylthiazol-5- yl)phenyl)sulfonyl)methyl)prop-2-en-1- amine |
| 18 | 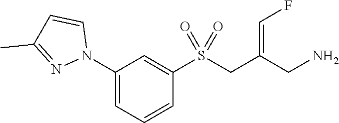 [SMILES: Cc1ccn(-c2cccc(S(=O)(=O)C/C(=C/F)CN)c2)n1] | (E)-3-fluoro-2-(((3-(3-methyl-1H- pyrazol-1- yl)phenyl)sulfonyl)methyl)prop-2-en-1- amine |
| 19 | 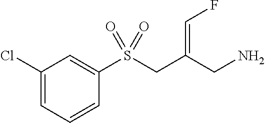 [SMILES: NC/C(=C\F)CS(=O)(=O)c1cccc(Cl)c1] | (E)-2-(((3-chlorophenyl)sulfonyl)methyl)- 3-fluoroprop-2-en-1-amine |
| 20 | 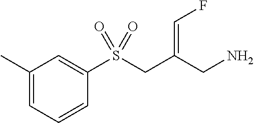 [SMILES: Cc1cccc(S(=O)(=O)C/C(=C/F)CN)c1] | (E)-3-fluoro-2-((m- tolylsulfonyl)methyl)prop-2-en-1-amine |
| 21 | 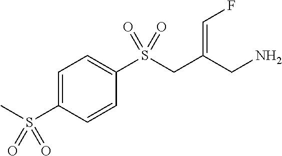 [SMILES: CS(=O)(=O)c1ccc(S(=O)(=O)C/C(=C/F)CN)cc1] | (E)-3-fluoro-2-(((4- (methylsulfonyl)phenyl)sulfonyl)methyl) prop-2-en-1-amine |
| 22 | 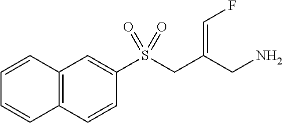 [SMILES: NC/C(=C\F)CS(=O)(=O)c1ccc2ccccc2c1] | (E)-3-fluoro-2-((naphthalen-2- ylsulfonyl)methyl)prop-2-en-1-amine |
| 23 | 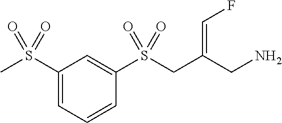 [SMILES: CS(=O)(=O)c1cccc(S(=O)(=O)C/C(=C/F)CN)c1] | (E)-3-fluoro-2-(((3- (methylsulfonyl)phenyl)sulfonyl)methyl) prop-2-en-1-amine |
| 24 | 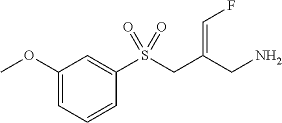 [SMILES: COc1cccc(S(=O)(=O)C/C(=C/F)CN)c1] | (E)-3-fluoro-2-(((3- methoxyphenyl)sulfonyl)methyl)prop-2- en-1-amine |
| 25 | 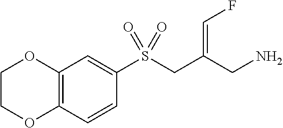 [SMILES: NC/C(=C\F)CS(=O)(=O)c1ccc2c(c1)OCCO2] | (E)-2-(((2,3-dihydrobenzo[b][1,4]dioxin- 6-yl)sulfonyl)methyl)-3-fluoroprop-2-en- 1-amine |
| 26 | 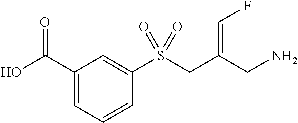 [SMILES: NC/C(=C\F)CS(=O)(=O)c1cccc(C(=O)O)c1] | (E)-3-((2-(aminomethyl)-3- fluoroallyl)sulfonyl)benzoic acid |
| 27 | 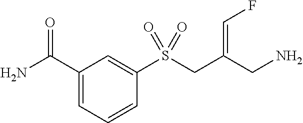 [SMILES: NC/C(=C\F)CS(=O)(=O)c1cccc(C(N)=O)c1] | (E)-3-((2-(aminomethyl)-3- fluoroallyl)sulfonyl)benzamide |
| 28 | 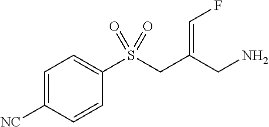 [SMILES: N#Cc1ccc(S(=O)(=O)C/C(=C/F)CN)cc1] | (E)-4-((2-(aminomethyl)-3- fluoroallyl)sulfonyl)benzonitrile |
| 29 | 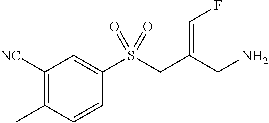 [SMILES: Cc1ccc(S(=O)(=O)C/C(=C/F)CN)cc1C#N] | (E)-5-((2-(aminomethyl)-3- fluoroallyl)sulfonyl)-2-methylbenzonitrile |
| 30 | 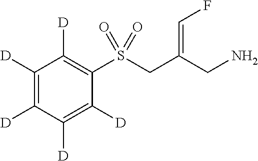 [SMILES: [2H]c1c([2H])c([2H])c(S(=O)(=O)C/C(=C/F)CN)c([2H])c1[2H]] | (E)-3-fluoro-2-(((phenyl- d₅)sulfonyl)methyl)prop-2-en-1-amine |
| 31 | 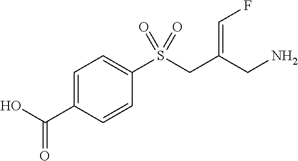 [SMILES: NC/C(=C\F)CS(=O)(=O)c1ccc(C(=O)O)cc1] | (E)-4-((2-(aminomethyl)-3- fluoroallyl)sulfonyl)benzoic acid |
| 32 | 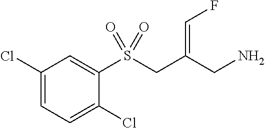 [SMILES: NC/C(=C\F)CS(=O)(=O)c1cc(Cl)ccc1Cl] | (E)-2-(((2,5- dichlorophenyl)sulfonyl)methyl)-3- fluoroprop-2-en-1-amine |
| 33 | 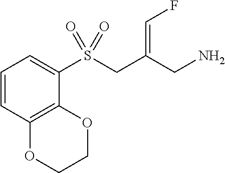 [SMILES: NC/C(=C\F)CS(=O)(=O)c1cccc2c1OCCO2] | (E)-2-(((2,3-dihydrobenzo[b][1,4]dioxin- 5-yl)sulfonyl)methyl)-3-fluoroprop-2-en- 1-amine |
| 34 | 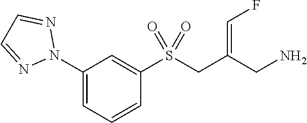 [SMILES: NC/C(=C\F)CS(=O)(=O)c1cccc(-n2nccn2)c1] | (E)-2-(((3-(2H-1,2,3-triazol-2- yl)phenyl)sulfonyl)methyl)-3-fluoroprop- 2-en-1-amine |
| 35 | 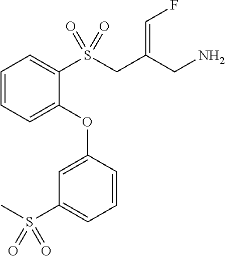 [SMILES: CS(=O)(=O)c1cccc(Oc2ccccc2S(=O)(=O)C/C(=C/F)CN)c1] | (E)-3-fluoro-2-(((2-(3- (methylsulfonyl)phenoxy)phenyl)sulfonyl) methyl)prop-2-en-1-amine |
| 36 | 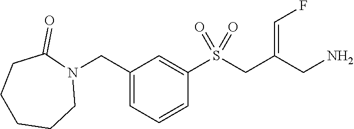 [SMILES: NC/C(=C\F)CS(=O)(=O)c1cccc(CN2CCCCCC2=O)c1] | (E)-1-(3-((2-(aminomethyl)-3- fluoroallyl)sulfonyl)benzyl)azepan-2-one |
| 37 | 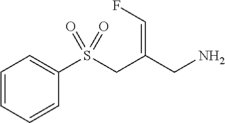 [SMILES: NC/C(=C/F)CS(=O)(=O)c1ccccc1] | (Z)-3-fluoro-2- ((phenylsulfonyl)methyl)prop-2-en-1- amine |

or a pharmaceutically acceptable salt thereof.

9. The compound according to claim 1 of the formula: 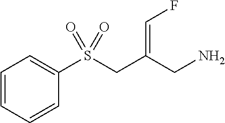 [SMILES: NC/C(=C\F)CS(=O)(=O)c1ccccc1]
or a pharmaceutically acceptable salt thereof.

10. The compound of claim 9 which is the hydrochloride salt:

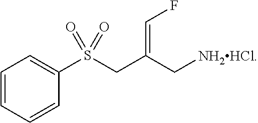

11. A pharmaceutical composition comprising a compound or a pharmaceutically acceptable salt thereof according to claim 1, and at least one pharmaceutically acceptable excipient, carrier or diluent.

12. A method of inhibiting the amine oxidase activity of any one of LOX, LOXL1, LOXL2, LOXL3 or LOXL4 in a subject in need thereof, comprising administering to the subject an effective amount of the compound or the pharmaceutically acceptable salt thereof according to claim 1.

13. A method of treating a condition by inhibiting the activity of any one of the LOX, LOXL1, LOXL2, LOXL3 and LOXL4 proteins, comprising administering to a subject in need thereof a therapeutically effective amount of the compound or the pharmaceutically acceptable salt thereof according to claim 1,
wherein the condition is selected from the group consisting of fibrosis, cancer, myeloid malignancy, and scarring.

14. (canceled)

15. The method of claim 13, wherein in a case that the condition is fibrosis, the fibrosis is selected from the group consisting of mediastinal fibrosis, myelofibrosis, retroperitoneal fibrosis, progressive massive fibrosis, nephrogenic systemic fibrosis, Crohn's Disease, keloid, scleroderma/systemic sclerosis, arthrofibrosis, Dupuytren's contracture, adhesive capsulitis, fibrosis of the pancreases, fibrosis of the intestine, liver fibrosis, lung fibrosis, kidney fibrosis, cardiac fibrosis, fibrostenosis, cystic fibrosis, idiopathic pulmonary fibrosis, radiation-induced fibrosis, Peyronie's disease and scleroderma or is associated with respiratory disease, abnormal wound healing and repair, scarring, hypertrophic scarring, scarring post-surgery, cardiac arrest and all conditions where excess or aberrant deposition of fibrous material is associated with disease, injury, implants or surgery;
wherein in a case that the condition is cancer, the cancer is selected from the group consisting of lung cancer; breast cancer; colorectal cancer; anal cancer; pancreatic cancer; prostate cancer; ovarian carcinoma; liver and bile duct carcinoma; esophageal carcinoma; mesothelioma, non-Hodgkin's lymphoma; bladder carcinoma; carcinoma of the uterus; glioma, glioblastoma, medullablastoma, and other tumours of the brain; myelofibrosis, kidney cancer; cancer of the head and neck; cancer of the stomach; multiple myeloma; testicular cancer; germ cell tumour; neuroendocrine tumour; cervical cancer; oral cancer, carcinoids of the gastrointestinal tract, breast, and other organs; signet ring cell carcinoma; mesenchymal tumours including sarcomas, fibrosarcomas, haemangioma, angiomatosis, haemangiopericytoma, pseudoangiomatous stromal hyperplasia, myofibroblastoma, fibromatosis, inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma or a leiomysarcoma; and
wherein in a case that the condition is a myeloid malignancy, the myeloid malignancy is selected from the group consisting of myelodysplastic syndromes (MDS), myelodysplastic neoplasma (MPN), MDS/MPS overlap syndromes and acute myeloid leukemia (AML), including acute promyelocytic leukemia (APL).

16. The method according to claim 13, wherein the condition is scarring.

17. A method of treating or preventing keloid diseases or scars, comprising administering to a subject in need thereof a therapeutically effective amount of the compound or the pharmaceutically acceptable salt thereof according to claim 1.

18. The method of claim 17, wherein the keloid diseases or scars are keloids, hypertrophic scars, postoperative scars, burn scars, posttraumatic scars, Dupuytren's contracture or are caused by external injuries or surgical procedures or are a result of a skin injury caused by acne, burns, chicken pox, infection, piercing, scratches, surgical cuts or vaccination sites.

19. The method of claim 17, wherein the treatment assists in wound healing and improves the skin compliance or skin appearance of the subject.

20. The method according to claim 12, wherein the compound is administered topically.

21. The method according to claim 12, wherein the compound is administered orally.

22. (canceled)
